(19) United States
(12) Patent Application Publication
(10) Pub. No.: US 2025/0110406 A1
(43) Pub. Date: Apr. 3, 2025

(54) METHOD OF PRODUCING RESIST COMPOSITION PURIFIED PRODUCT, RESIST PATTERN FORMING METHOD, AND RESIST COMPOSITION PURIFIED PRODUCT

(71) Applicant: TOKYO OHKA KOGYO CO., LTD., Kanagawa (JP)

(72) Inventors: Takuma KANAMARU, Kanagawa (JP); Kosuke WATAHIKI, Kanagawa (JP); Satoshi AKIYAMA, Kanagawa (JP)

(21) Appl. No.: 18/894,241

(22) Filed: Sep. 24, 2024

(30) Foreign Application Priority Data
Oct. 2, 2023 (JP) 2023-171303

(51) Int. Cl. G03F 7/039; G03F 7/004; G03F 7/075

(52) U.S. Cl. CPC G03F 7/0397; G03F 7/0045; G03F 7/0757

(57) ABSTRACT

A method of producing a resist composition purified product, the method including a step of filtering a resist composition through a filter having a porous structure in which adjacent spherical cells communicate with each other, in which the filter includes a porous membrane containing at least one resin selected from the group consisting of polyimide and polyamide-imide, the resist composition contains a resin component (A1) whose solubility in a developing solution is changed by an action of an acid and an organic solvent component, and the resin component (A1) contains a copolymer having a constitutional unit (a01) with an onium salt structure that generates sulfonic acid upon light exposure and a constitutional unit (a02) with an onium salt structure that generates a carboxylic acid upon light exposure.

Priority is claimed on Japanese Patent Application No. 2023-171303, filed Oct. 2, 2023, the content of which is incorporated herein by reference.

### BACKGROUND OF THE INVENTION

### Field of the Invention

The present invention relates to a method of producing a resist composition purified product, a resist pattern forming method, and a resist composition purified product.

### Description of Related Art

In lithography techniques, for example, a step of forming a resist pattern having a predetermined shape on a resist film is performed by forming a resist film formed of a resist material on a substrate, and selectively exposing the resist film to light and performing a developing treatment thereon.

In recent years, in the manufacture of semiconductor elements and liquid crystal display elements, advances in lithography techniques have led to rapid progress in the field of pattern miniaturization. These pattern fining techniques typically involve shortening the wavelength (increasing the energy) of the exposure light source. Specifically, although ultraviolet rays typified by g-rays and i-rays have been used in the related art, semiconductor elements are mass-produced using KrF excimer lasers or ArF excimer lasers at present, and development using extreme ultraviolet rays (EUV), electron beams (EB), X-rays, or the like having shorter wavelengths (higher energy) than these excimer lasers has also been performed.

A resist material is required to have lithography characteristics such as resolution that enables reproduction of a fine-sized pattern, and sensitivity to these types of exposure light sources. As a resist material that satisfies these requirements, a chemically amplified resist composition containing a base material component whose solubility in a developing solution is changed by an action of an acid and an acid generator component that generates an acid upon light exposure has been used. In the chemically amplified resist composition, a resin having a plurality of constitutional units is used as the base material component. Known examples of the acid generator component include an onium salt-based acid generator such as an iodonium salt or a sulfonium salt, an oxime sulfonate-based acid generator, a diazomethane-based acid generator, a nitrobenzylsulfonate-based acid generator, an iminosulfonate-based acid generator, and a disulfone-based acid generator.

Meanwhile, as the pattern fining progresses, the influence of impurities present in the resist material is likely to be apparent in the pattern formation. On the other hand, in the production of a resist composition, in general, purification is performed by passing the composition through a filter in order to remove impurities in the composition (for example, see Patent Document 1).

### CITATION LIST

### Patent Literature

[Patent Document 1] Japanese Patent No. 7195418

### SUMMARY OF THE INVENTION

### Technical Problem

With further advances in lithography technologies, rapid progress in the field of pattern fining has been achieved together with the expansion of application fields. In association with this, a technique of forming a pattern with fine dimensions in a satisfactory shape is required in the manufacture of a semiconductor element or the like. Therefore, the resist material is required to have even higher sensitivity and improved lithography characteristics. In response to this requirement, examination has been conducted on utilizing a resin in which a constitutional unit that generates an acid upon light exposure is introduced as a base material component of a chemically amplified resist composition. Meanwhile, in a case of forming a resist pattern with dimensions of tens of nanometers, a problem of occurrence of defects during the formation of a resist film has been significant.

The present invention has been made in consideration of the above-described circumstances, and an object thereof is to provide a method of producing a resist composition purified product in which impurities are further reduced and occurrence of defects during formation of a resist film can be reduced, a resist composition purified product produced by the production method, and a resist pattern forming method in which a resist pattern is formed using the resist composition purified product.

### Solution to Problem

In order to solve the above-described problems, the present invention has adopted the following configurations.

According to a first aspect of the present invention, there is provided a method of producing a resist composition purified product, the method including: a step (i) of filtering a resist composition through a filter having a porous structure in which adjacent spherical cells communicate with each other, in which the filter includes a porous membrane containing at least one resin selected from the group consisting of polyimide and polyamide-imide, the resist composition contains a resin component whose solubility in a developing solution is changed by an action of an acid, and an organic solvent component, the resin component contains a copolymer having a constitutional unit (a01) with an onium salt structure that generates sulfonic acid upon light exposure and a constitutional unit (a02) with an onium salt structure that generates a carboxylic acid upon light exposure.

According to a second aspect of the present invention, there is provided a resist pattern forming method including a step of obtaining a resist composition purified product using the method of producing a resist composition purified product according to the first aspect; a step of forming a resist film on a support using the resist composition purified product; a step of exposing the resist film to light; and a step of developing the resist film exposed to light to form a resist pattern.

According to a third aspect of the present invention, there is provided a resist composition purified product including: a resin component whose solubility in a developing solution is changed by an action of an acid and an organic solvent component, in which the resin component contains a copolymer having a constitutional unit (a01) with an onium salt structure that generates sulfonic acid upon light exposure and a constitutional unit (a02) with an onium salt structure that generates a carboxylic acid upon light exposure, and the number of counting target objects with a size of 0.135 μm or greater, which is counted by a light scattering type liquid-borne particle counter, is 0.1 particles/mL or less.

According to a fourth aspect of the present invention, there is provided a resist pattern forming method including a step of forming a resist film on a support using the resist composition purified product according to the third aspect; a step of exposing the resist film to light; and a step of developing the resist film exposed to light to form a resist pattern.

### Advantageous Effects of Invention

According to the present invention, it is possible to provide a method of producing a resist composition purified product in which impurities are further reduced and occurrence of defects during formation of a resist film can be reduced, a resist composition purified product produced by the production method, and a resist pattern forming method in which a resist pattern is formed using the resist composition purified product.

### BRIEF DESCRIPTION OF THE DRAWINGS

FIG. 1 is a view schematically showing an embodiment of a communication pore constituting a polyimide-based resin porous membrane.

### DETAILED DESCRIPTION OF THE INVENTION

In the present specification and the scope of the present claims, the term “aliphatic” is a relative concept used with respect to “aromatic” and defines a group or compound that has no aromaticity.

The term “alkyl group” includes a linear, branched, or cyclic monovalent saturated hydrocarbon group unless otherwise specified. The same applies to the alkyl group in an alkoxy group.

The term “alkylene group” includes a linear, branched chain-like, or cyclic divalent saturated hydrocarbon group unless otherwise specified.

The “halogenated alkyl group” is a group in which some or all hydrogen atoms of an alkyl group have been substituted with halogen atoms. Examples of the halogen atom include a fluorine atom, a chlorine atom, a bromine atom, and an iodine atom.

The “fluorinated alkyl group” or a “fluorinated alkylene group” is a group in which some or all hydrogen atoms of an alkyl group or an alkylene group have been substituted with fluorine atoms.

The term “constitutional unit” indicates a monomer unit constituting a polymer compound (a resin, a polymer, or a copolymer).

The expression “may have a substituent” includes a case where a hydrogen atom (—H) is substituted with a monovalent group and a case where a methylene (—CH₂—) group is substituted with a divalent group.

The term “light exposure” is a general concept for irradiation with radiation.

The term “polyimide-based resin” denotes one or both of polyimide and polyamide-imide. The polyimide and the polyamide-imide may each have at least one functional group selected from the group consisting of a carboxy group, a salt type carboxy group, and an —NH— bond.

A porous membrane containing at least one of polyimide and polyamide-imide may be referred to as “polyimide-based resin porous membrane”. A porous membrane containing polyimide may be referred to as “polyimide porous membrane”. A porous membrane containing polyamide-imide may be referred to as “polyamide-imide porous membrane”.

The term “acid decomposable group” indicates a group having acid decomposability in which at least a part of a bond in the structure of the acid decomposable group can be cleaved by the action of an acid.

Examples of the acid decomposable group whose polarity is increased by the action of an acid include groups which are decomposed by the action of an acid to generate a polar group.

Examples of the polar group include a carboxy group, a hydroxyl group, an amino group, and a sulfo group (—SO₃H).

More specific examples of the acid decomposable group include a group in which the above-described polar group has been protected by an acid dissociable group (such as a group in which a hydrogen atom of the OH-containing polar group has been protected by an acid dissociable group).

Here, the term “acid dissociable group” indicates both a group (i) having an acid dissociation property in which a bond between the acid dissociable group and an atom adjacent to the acid dissociable group can be cleaved by the action of an acid and a group (ii) in which some bonds are cleaved by the action of an acid, a decarboxylation reaction occurs, and thus the bond between the acid dissociable group and the atom adjacent to the acid dissociable group can be cleaved.

It is necessary that the acid dissociable group that constitutes the acid decomposable group is a group which exhibits a lower polarity than that of the polar group generated by the dissociation of the acid dissociable group. Thus, in a case where the acid dissociable group is dissociated by the action of an acid, a polar group exhibiting a higher polarity than that of the acid dissociable group is generated so that the polarity is increased. As a result, the polarity of an entire component (A1) is increased. Due to the increase in the polarity, the solubility in a developing solution is relatively changed such that the solubility is increased in a case where the developing solution is an alkali developing solution and the solubility is decreased in a case where the developing solution is an organic developing solution.

The term “base material component” denotes an organic compound having a film-forming ability. Organic compounds used as the base material component are classified into non-polymers and polymers. As the non-polymers, typically non-polymers having a molecular weight of 500 or greater and less than 4000 (hereinafter, referred to as “low-molecular-weight compounds”) are used. Hereinafter, “resin”, “polymer compound”, or “polymer” indicates a polymer having a molecular weight of 1000 or greater. As the molecular weight of the polymer, the weight-average molecular weight in terms of polystyrene according to gel permeation chromatography (GPC) is used.

The expression “constitutional unit to be derived” denotes a constitutional unit formed by cleavage of a multiple bond between carbon atoms, for example, an ethylenic double bond.

In “acrylic acid ester”, the hydrogen atom bonded to the carbon atom at the α-position may be substituted with a substituent. The substituent (R^{αx}) that substitutes the hydrogen atom bonded to the carbon atom at the α-position is an atom other than the hydrogen atom or a group. Further, itaconic acid diester in which the substituent (R^{αx}) has been substituted with a substituent having an ester bond or α-hydroxyacryl ester in which the substituent (R^{αx}) has been substituted with a hydroxyalkyl group or a group obtained by modifying a hydroxyl group thereof can be described as acrylic acid ester. Further, the carbon atom at the α-position of acrylic acid ester indicates the carbon atom to which the carbonyl group of acrylic acid is bonded, unless otherwise specified. Hereinafter, acrylic acid ester in which the hydrogen atom bonded to the carbon atom at the α-position has been substituted with a substituent is also referred to as α-substituted acrylic acid ester.

The concept “derivative” includes those obtained by substituting a hydrogen atom at the α-position of a target compound with another substituent such as an alkyl group or a halogenated alkyl group, and derivatives thereof. Examples of the derivatives thereof include a derivative in which the hydrogen atom of the hydroxyl group of the object compound in which the hydrogen atom at the α-position may be substituted with a substituent is substituted with an organic group; and a derivative in which a substituent other than a hydroxyl group is bonded to the object compound in which the hydrogen atom at the α-position may be substituted with a substituent. In addition, the α-position denotes the first carbon atom adjacent to the functional group unless otherwise specified.

Examples of the substituent that substitutes a hydrogen atom at the α-position of hydroxystyrene include the same groups as those for R^{αx}, such as an alkyl group and a halogenated alkyl group.

In the present specification and the scope of the claims, asymmetric carbons may be present and enantiomers or diastereomers may be present depending on the structures of the chemical formulae. In this case, these isomers are represented by one chemical formula. These isomers may be used alone or in the form of a mixture.

### (Production Method for Resist Composition Purified Product)

A method of producing a resist composition purified product according to a first aspect of the present invention includes a step of filtering a resist composition through a filter having a porous structure in which adjacent spherical cells communicate with each other (hereinafter, this step will also be referred to as “step (i)”).

The filter has a porous membrane containing at least one resin selected from the group consisting of polyimide and polyamide-imide. The resist composition contains a resin component whose solubility in a developing solution is changed by an action of an acid and an organic solvent component. The resin component contains a copolymer having a constitutional unit (a01) with an onium salt structure that generates sulfonic acid upon light exposure and a constitutional unit (a02) with an onium salt structure that generates a carboxylic acid upon light exposure.

According to the step (i), impurities such as particles are removed from the resist composition, and a high-purity resist composition purified product is obtained.

According to such a production method of the first aspect, in particular, components with high polarity and polymers, which have been difficult to remove from a resist composition in the related art, are sufficiently removed by using a filter having a porous structure in which adjacent spherical cells communicate with each other and including a porous membrane that contains at least one resin selected from the group consisting of polyimide and polyamide-imide, and among these, polymers with high polarity are specifically removed. In a case where the resist composition as a filtration target contains a copolymer having the constitutional unit (a01) and the constitutional unit (a02), components with higher polarity coexist as impurities.

In addition, a metal component as an impurity is also sufficiently removed from the resist composition in the step (i). This metal component may be originally contained in the component constituting the resist composition, but may also be mixed from a resist composition transfer path such as a pipe or a joint of a production device. In the step (i), it is possible to effectively remove, for example, iron, nickel, zinc, chromium, and the like, which are easily mixed from a production device or the like.

<Step (i)>

The step (i) is a step of filtering the resist composition through a filter having a porous structure in which adjacent spherical cells communicate with each other.

### <<Filter>>

The filter that is used in the present step has a porous structure in which adjacent spherical cells communicate with each other.

For example, such a filter may be formed of a single substance of a porous membrane in which adjacent spherical cells communicate with each other, or may be a filter in which another filter medium is used together with the porous membrane.

Examples of other filter media include a nylon membrane, a polytetrafluoroethylene membrane, a tetrafluoroethylene/perfluoroalkyl vinyl ether copolymer (PFA) membrane, and a membrane obtained by modifying these membranes.

In such a filter, regions of the porous membrane before and after a liquid passes therethrough are preferably sealed so that a supply liquid and a filtrate of the resist composition are separated without being mixed. Examples of the method for this sealing include a method of processing the porous membrane by adhesion with light (UV) curing, adhesion with heat (including adhesion by an anchoring effect (heat welding or the like)), adhesion using an adhesive, or the like, and a method of carrying out processing by adhering the porous membrane and another filter medium by an assembling method or the like. Examples of such a filter include those in which such a porous membrane described above is provided in an outer container made of a thermoplastic resin (polyethylene, polypropylene, PFA, polyether sulfone (PES), polyimide, polyamide-imide, or the like).

Examples of the form of the porous membrane in such a filter include a planar shape and a pipe shape in which opposite sides of a porous membrane are merged. The surface of the pipe-shaped porous membrane is preferably pleated from the viewpoint of increasing the area that comes into contact with the supply liquid.

In regard to “porous membrane in which adjacent spherical cells communicate with each other” The “porous membrane in which adjacent spherical cells communicate with each other” provided in such a filter has a communication pore in which adjacent spherical cells communicate with each other.

The communication pore is formed of individual pores (cells) that impart porosity to the porous membrane. Such a pore includes a pore in which almost the entire inner surface of the pore is a curved surface, and may include a pore having a shape other than this shape.

In the present specification, a pore in which almost the entire inner surface of the pore is a curved surface is referred to as “spherical cell” or “substantially spherical pore”. In the spherical cell (the substantially spherical pore), the inner surface of the pore forms a substantially spherical space. The spherical cell is easily formed in a case where fine particles that are used in the production method for a polyimide-based resin porous membrane described below are substantially spherical.

“Substantially spherical” is a concept including a true sphere. However, it is not necessarily limited to the true sphere and is a concept including a substantially spherical one. “Substantially spherical” means that a sphericity defined by a ratio of a major axis to a minor axis, which is represented by a value obtained by dividing the major axis by the minor axis of the particle, is within 1±0.3. Here, in the spherical cell, such a sphericity is preferably within 1±0.1 and more preferably within 1±0.05. In the porous membrane in which adjacent spherical cells communicate with each other, the adjacent spherical cells form at least a part of the communication pore.

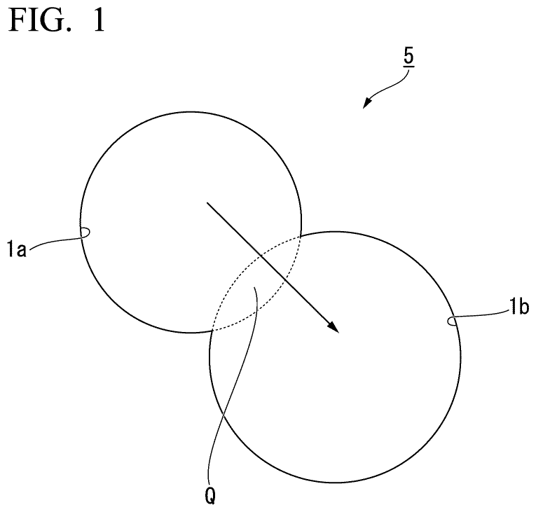

FIG. 1 is a view schematically showing an embodiment of a communication pore constituting a porous membrane.

In each of a spherical cell 1a and a spherical cell 1b in the porous membrane according to the present embodiment, almost the entire inner surface is a curved surface, and a substantially spherical space is formed.

The spherical cell 1a and the spherical cell 1b are adjacent to each other, and a communication pore 5 in which an overlapping portion Q of the spherical cell 1a and the spherical cell 1b adjacent to each other penetrates between the cells is formed. A filtration target flows through the communication pore 5, for example, in the direction (the arrow direction) from the spherical cell 1a toward the spherical cell 1b.

As described above, in the porous membrane having a structure in which adjacent spherical cells communicate with each other, it is preferable that a plurality of pores (spherical cells or communication pores) are connected to form a flow path of a filtration target as a whole.

The “flow path” is generally formed by the individual “pores” and/or “communication pores” that communicate with each other. The individual pores are formed, for example, by removing individual fine particles present in a polyimide-based resin-fine particle composite membrane in a subsequent step in the production method for a polyimide-based resin porous membrane described below. In addition, the communication pore is pores adjacent to each other, which are formed in a portion where individual fine particles have been in contact with each other, where the portion is present in the polyimide-based resin-fine particle composite membrane, by removing the fine particles in a subsequent step in the production method for a polyimide-based resin porous membrane described below.

In the porous membrane, a spherical cell and a communication pore in which adjacent spherical cells communicate with each other are formed, and thus the degree of porosity is increased. Further, in the porous membrane, a spherical cell or a communication pore opens on a surface of the porous membrane, and a communication pore that opens on one surface is connected to the inside of the porous membrane and opens on the other (on the back side) surface, and thus a flow path in which a fluid can pass through the inside of the porous membrane is formed. In addition, according to the porous membrane, in a case where a filtration target flows through the flow path, foreign substances contained in a filtration target are removed from the filtration target before filtration.

Since the porous membrane has a flow path obtained by causing communication pores formed by spherical cells having a curved surface on the inner surface thereof to be continuous, the surface area of the inner surface of the spherical cells is large. As a result, not only a filtration target can pass through the inside of the porous membrane but also the frequency of contacting with the inner surface of the spherical cell increases in a case where the filtration target passes while coming into contact with the curved surfaces of the individual spherical cells, and thus foreign substances that are present in the filtration target are adsorbed by the inner surface of the spherical cell, and the foreign substances are easily removed from the filtration target.

The porous membrane preferably has a structure in which spherical cells having an average pore diameter of 10 to 500 nm communicate with each other. The average pore diameter of the spherical cells is preferably in a range of 10 to 2500 nm, more preferably in a range of 20 to 1000 nm, still more preferably in a range of 30 to 500 nm, and particularly preferably in a range of 40 to 400 nm.

The flow path included in the inside of “porous membrane in which adjacent spherical cells communicate with each other” may have, in addition to the above-described spherical cells and the communication pores between the spherical cells, pores having other shapes or communication pores including these pores.

In addition, the spherical cell may further have a recessed part on the inner surface thereof. For example, a pore having a pore diameter less than that of the spherical cell may be formed in the recessed part, where the pore opens on the inner surface of the spherical cell.

Examples of “porous membrane in which adjacent spherical cells communicate with each other” include those containing a resin, which may be substantially formed of only a resin, and include those in which the resin in the entire porous membrane is preferably 95% by mass or greater, more preferably 98% by mass or greater, and still more preferably 99% by mass or greater.

The porous membrane contains a polyimide-based resin. A porous membrane containing a polyimide-based resin is excellent in foreign substance removing properties and strength, and stability of lithography characteristics before and after filtration. The porous membrane contains at least one of polyimide and polyamide-imide as a resin and preferably at least polyimide. The porous membrane may contain only polyimide as a resin or only polyamide-imide, but it is preferable that the porous membrane contains only polyimide.

It is particularly preferable that in “porous membrane in which adjacent spherical cells communicate with each other”, 95% by mass or greater of the entire porous membrane is at least one of polyimide and polyamide-imide. That is, it is preferable that the filter includes a porous membrane (polyimide-based resin porous membrane) which contains a polyimide-based resin as a resin and in which adjacent spherical cells communicate with each other.

### Polyimide-Based Resin Porous Membrane

The polyimide-based resin may have at least one functional group selected from the group consisting of a carboxy group, a salt type carboxy group, and a —NH— bond.

The polyimide-based resin preferably has the above-described functional group in a moiety other than the terminal of the main chain. Preferred examples having the above-described functional group in a moiety other than the terminal of the main chain include a polyamic acid.

In the present specification, “salt type carboxy group” means a group obtained by substituting a hydrogen atom in a carboxy group with a cation component. “Cation component” may be a cation itself in a state of being completely ionized, may be a cation constitutional element in a state of being ionically bonded to —COO— and virtually uncharged, and may be a partially charged cation constitutional element having a partial charge in a state of being an intermediate state between the two described above.

In a case where “cation component” is an M ion component consisting of an n-valent metal M, the cation itself is represented by M^{m+}, and the cation constitutional element is an element represented by “M_{1/n}” in “—COOM_{1/n}”.

Examples of “cation component” include a cation in a case where a compound mentioned as a compound contained in an etching liquid described below is ion-dissociated. Representative examples thereof include an ion component and an organic alkali ion component. For example, in a case where the alkali metal ion component is a sodium ion component, the cation itself is a sodium ion (Na⁺), and the cation constitutional element is an element represented by “Na” in “—COONa”. The partially charged cation constitutional element is Na^{δ+}.

The cation component is not particularly limited, and examples thereof include an inorganic component such as NH₄⁺ and an organic component such as N(CH₃)₄⁺. Examples of the inorganic component include alkali metals such as Li, Na, and K; and metal elements such as alkaline earth metals such as Mg and Ca. Examples of the organic component include an organic alkali ion component. Examples of the organic alkali ion component include quaternary ammonium cations represented by NH₄⁺, for example, NR₄⁺ (all four R's represent organic groups and may be the same as or different from each other). The organic group as R represents preferably an alkyl group and more preferably an alkyl group having 1 to 6 carbon atoms. Examples of the quaternary ammonium cation include N(CH₃)₄⁺.

The state of the cation component in the salt type carboxy group is not particularly limited, and generally depends on the environment in which the polyimide-based resin is present, for example, an environment in an aqueous solution, an environment in an organic solvent, and a dry environment. In a case where the cation component is a sodium ion component, for example, there is a possibility that —COO⁻ and Na⁺ are dissociated in a case of being in an aqueous solution, and there is a high possibility that —COONa is not dissociated in a case of being in an organic solvent or being dried.

The polyimide-based resin may have at least one functional group selected from the group consisting of a carboxy group, a salt type carboxy group, and an —NH— bond; however, in a case of having at least one of these, it generally has both a carboxy group and/or a salt type carboxy group and a —NH— bond. The polyimide-based resin may have only a carboxy group, may have only a salt type carboxy group, or may have both a carboxy group and a salt type carboxy group, in a case where the carboxy group and/or the salt type carboxy group is concerned. The ratio between the carboxy group and the salt type carboxy group contained in the polyimide-based resin may vary, for example, depending on the environment in which the polyimide-based resin is present, even in a case where the polyimide-based resin is the same, and it is also affected by the concentration of the cation component.

In the case of polyimide, the total of the number of moles of the carboxy group and the salt type carboxy group contained in the polyimide-based resin is generally equimolar to that of the —NH— bond.

In particular, in the production method for a polyimide porous membrane described below, in a case where a carboxy group and/or a salt type carboxy group is formed of part of imide bonds in the polyimide, an —NH— bond is also formed substantially at the same time. The total of the numbers of moles of the carboxy group to be formed and the salt type carboxy group to be formed is equimolar to that of the —NH— bond formed.

In the case of the production method for a porous polyamide-imide membrane, the total of the number of moles of the carboxy group and the salt type carboxy group in the polyamide-imide is not necessarily equimolar to that of the —NH— bond, and it depends on the conditions for chemical etching or the like in the etching (the decyclization of the imide bond) step described below.

The polyimide-based resin preferably has, for example, at least one constitutional unit selected from the group consisting of constitutional units each represented by General Formulae (1) to (4).

In the case of polyimide, it is preferable to contain at least one constitutional unit selected from the group consisting of a constitutional unit represented by General Formula (1) and a constitutional unit represented by General Formula (2).

In the case of polyamide-imide, it is preferable to contain at least one constitutional unit selected from the group consisting of a constitutional unit represented by General Formula (3) and a constitutional unit represented by General Formula (4).

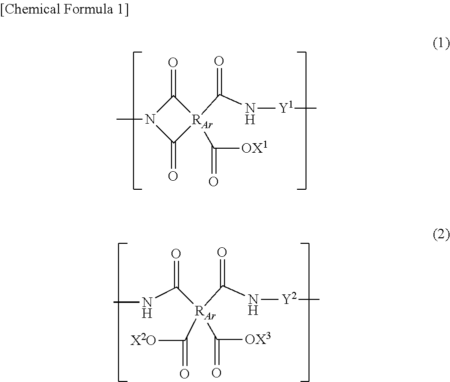

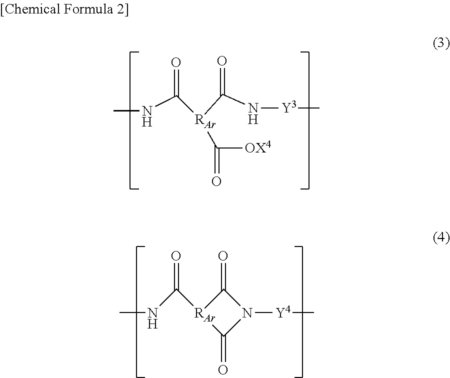

In Formulae (1) to (3), X¹ to X⁴ may be the same as or different from each other, and are a hydrogen atom or a cation component.

R_{Ar} represents an aryl group, and examples thereof include the same groups as those for the aryl group represented by RA, to which a carbonyl group is bonded, in each of a constitutional unit represented by General Formula (5) constituting a polyamic acid described below or a constitutional unit represented by General Formula (6) constituting aromatic polyimide.

Y¹ to Y⁴ each independently represent a divalent residue excluding the amino group of the diamine compound, and examples thereof include the same groups as those for the arylene group represented by R′_{Ar}, to which N is bonded, in each of a constitutional unit represented by General Formula (5) constituting a polyamic acid described below or a constitutional unit represented by General Formula (6) constituting aromatic polyimide.

The polyimide-based resin may be a resin in which a part of an imide bond (—N[—C(=O)]₂) of typical polyimide or polyamide-imide is decyclized and has each of the constitutional unit represented by General Formula (1) or General Formula (2) in a case of polyimide and the constitutional unit represented by General Formula (3) in a case of polyamide-imide.

The polyimide-based resin porous membrane may contain a polyimide-based resin obtained by decyclizing part of imide bonds, thereby having at least one functional group selected from the group consisting of a carboxy group, a salt type carboxy group, and an —NH— bond.

The non-change rate in a case where part of imide bonds are decyclized is determined by the following procedures (1) to (3).

Procedure (1): For a polyimide-based resin porous membrane that does not undergo the etching (the decyclization of imide bond) step described below (however, in case where a varnish for producing the porous membrane contains a polyamic acid, it is assumed that the imidization reaction has been substantially completed in the step of sintering an unsintered composite membrane), an area of a peak that represents the imide bond, measured by a Fourier transform infrared spectroscopy (FT-IR) apparatus, is divided by an area of a peak that represents benzene, also measured by the transform infrared spectroscopy (FT-IR) apparatus, to determine a value (X01).

Procedure (2): For a polyimide-based resin porous membrane obtained by using the same polymer (the varnish) as that of the porous membrane from which the above value (X01) has been determined, where the polyimide-based resin porous membrane has undergone the etching (the decyclization of imide bond) step described below, an area of a peak that represents the imide bond, measured by a Fourier transform infrared spectroscopy (FT-IR) apparatus, is divided by an area of a peak that represents benzene, also measured by the Fourier transform infrared spectroscopy (FT-IR) apparatus, to determine a value represented by a value (X02).

Procedure (3): the non-change rate is calculated according to the following expression.

$$Non - change rate ( \% ) = ( X 02 ) / ( X 01 ) \times 100$$

The non-change rate in the polyimide-based resin porous membrane is preferably in a range of 60% or greater, more preferably in a range of 70% to 99.5%, and still more preferably in a range of 80% to 99%. In the case of a porous membrane containing polyamide-imide, the non-change rate may be 100% since a —NH— bond is contained.

In the case of a polyimide porous membrane, an area of a peak that represents the imide bond, measured by an FT-IR apparatus, is divided by an area of a peak that represents benzene, also measured by the FT-IR apparatus, to determine a value that is denoted by the “imidization rate”.

The imidization rate regarding the value (X02) determined in the above procedure (2) is preferably 1.2 or greater, more preferably in a range of 1.2 to 2, and still more preferably in a range of 1.3 to 1.6, particularly preferably in a range of 1.30 to 1.55, and most preferably 1.35 or greater and less than 1.5. In addition, the imidization rate regarding the value (X01) determined in the above procedure (1) is preferably 1.5 or greater.

As the numerical value of such an imidization rate becomes relatively larger, it means that the number of imide bonds becomes larger, that is, the number of decyclized imide bonds described above becomes smaller.

### Production Method for Polyimide-Based Resin Porous Membrane

The polyimide-based resin porous membrane can be produced by a method including a step (hereinafter, referred to as an “etching step”) of forming a carboxy group and/or a salt type carboxy group from part of imide bonds in polyimide and/or polyamide-imide.

In the etching step, in a case where a carboxy group and/or a salt type carboxy group is formed of part of imide bonds in the polyimide, an —NH— bond theoretically equimolar to these groups is also formed substantially at the same time.

In a case where the resin contained in the polyimide-based resin porous membrane is substantially made of polyamide-imide, the porous membrane already has a —NH— bond even without undergoing the etching step and exhibits good adsorption power to foreign substances in the filtration target. In such a case, the etching step is not always necessary since it is not needed to slow down the flow rate of the filtration target; however, it is preferable to provide the etching step from the viewpoint of more effectively achieving the object of the present invention.

In the production method for a polyimide-based resin porous membrane, it is preferable to carry out the etching step after preparing a molding membrane containing polyimide and/or polyamide-imide as a main component (hereinafter, may be abbreviated as a “polyimide-based resin molded membrane”).

The polyimide-based resin molded membrane to be subjected to the etching step may be porous or may be non-porous.

In addition, the form of the polyimide-based resin molded membrane is not particularly limited; however, it preferably has a thin shape such as a membrane, and it is more preferably porous and has a thin shape such as a membrane from the viewpoint that the degree of porosity in the polyimide-based resin porous membrane to be obtained can be increased.

As described above, the polyimide-based resin molded membrane may be non-porous in a case where the etching step is carried out; however, in that case, it is preferable to make the polyimide-based resin molded membrane porous after the etching step.

The method for making the polyimide-based resin molded membrane porous before or after the etching step is preferably a method including a [fine particle removal] step of removing fine particles from a composite membrane (hereinafter, referred to as a “polyimide-based resin-fine particle composite membrane”) of polyimide and/or polyamide-imide and fine particles, to make the composite membrane porous.

Examples of the production method for a polyimide-based resin porous membrane include the following production method (a) and production method (b).

The production method (a): a method of etching a composite membrane of polyimide and/or polyamide-imide and fine particles before a [fine grain removal] step

The production method (b): A method of carrying out an etching step, after the [Fine particle removal] step, on a polyimide-based resin molded membrane made porous by the [Fine particle removal] step Among these, the latter production method (b) is preferable from the viewpoint that the degree of porosity in the polyimide-based resin porous membrane to be obtained can be further increased.

An example of the production method for a polyimide-based resin porous membrane will be described below.

### [Preparation of Varnish]

A fine particle dispersion liquid in which fine particles are dispersed in an organic solvent in advance is mixed with polyamic acid, or polyimide or polyamide-imide at any ratio or tetracarboxylic acid dianhydride and a diamine are polymerized to be polyamic acid in the above fine particle dispersion liquid, or further, the above polyamic acid is imidized to be polyimide, whereby a varnish is prepared.

The viscosity of the varnish is preferably in a range of 300 to 2,000 cP (0.3 to 2 Pa·s) and more preferably in a range of 400 to 1,800 cP (0.4 to 1.8 Pa·s). In a case where the viscosity of the varnish is within the above range, a membrane can be formed more uniformly.

The viscosity of the varnish can be measured with an E type rotational viscometer under a temperature condition of 25° C.

Resin fine particles are mixed with polyamic acid, or polyimide or polyamide-imide in the varnish so that the ratio of the fine particles/the polyimide-based resin is preferably in a range of 1 to 4 (mass ratio) and more preferably in a range of 1.1 to 3.5 (mass ratio) in a case of being sintered (dried in a case where sintering is optional) to form a polyimide-based resin-fine particle composite membrane.

In addition, fine particles are mixed with polyamic acid, or polyimide or polyamide-imide so that the volume fraction of the fine particles/the polyimide-based resin is preferably in a range of 1.1 to 5 and more preferably in a range of 1.1 to 4.5 in a case of being formed into a polyimide-based resin-fine particle composite membrane. In a case where the mass ratio or the volume fraction is greater than or equal to the preferable lower limits of the above-described ranges, pores having a suitable density as a porous membrane can be easily obtained, and in a case where it is less than or equal to than the preferable upper limits of the above-described ranges, problems such as the increase in viscosity and the cracking in the membrane hardly occur, and membrane formation can be stably achieved.

In addition, in the present specification, the volume fraction indicates a value obtained at 25° C.

### Fine Particles

As the fine particle material, any material can be used without particular limitation as long as it is insoluble in an organic solvent that is used for the varnish and can be selectively removed after the membrane formation.

Examples of the fine particle material include metal oxides such as silica (silicon dioxide), titanium oxide, alumina (Al₂O₃), and calcium carbonate as the inorganic materials. Examples of organic materials include organic polymers such as a high molecular weight olefin (polypropylene, polyethylene, or the like), polystyrene, an acrylic resin (methyl methacrylate, isobutyl methacrylate, polymethyl methacrylate (PMMA), or the like), an epoxy resin, cellulose, polyvinyl alcohol, polyvinyl butyral, polyester, polyether, and polyethylene.

Among the above, the inorganic material is preferably silica such as colloidal silica since micropores having a curved surface on the inner surface are easily be formed in the porous membrane. The organic material is preferably an acrylic resin such as PMMA.

The resin fine particles can be selected from, for example, typical linear polymers and known depolymerizable polymers without particular limitation depending on the intended purpose. The typical linear polymer is a polymer in which the molecular chains of the polymer are randomly cleaved during thermal decomposition. The depolymerizable polymer is a polymer that decomposes into monomers during thermal decomposition. Any polymer can be removed from the polyimide-based resin membrane by being decomposed into monomers, low molecular weight substances, or CO₂ in a case of being heated.

Among the depolymerizable polymers, from the viewpoint of handling at the time of pore formation, a polymer of methyl methacrylate or isobutyl methacrylate alone (a polymethyl methacrylate or a polyisobutyl methacrylate), which has a low thermal decomposition temperature, or a copolymerization polymer containing this as a main component is preferable.

The decomposition temperature of the resin fine particles is preferably in a range of 200° C. to 320° C. and more preferably in a range of 230° C. to 260° C. In a case where the decomposition temperature is 200° C. or higher, membrane formation can be carried out even in a case where a high boiling point solvent is used for the varnish, and the range of selection of sintering conditions for the polyimide-based resin becomes wider. In a case where the decomposition temperature is 320° C. or lower, only the resin fine particles can be easily eliminated without causing thermal damage to the polyimide-based resin.

The fine particles preferably have a high true sphere ratio since they tend to have a curved surface on the inner surface of the pore in the formed porous membrane. The particle diameter (average diameter) of the fine particles to be used is, for example, preferably in a range of 20 to 2500 nm, more preferably in a range of 30 to 1000 nm, and still more preferably in a range of 50 to 250 nm.

In a case where the average diameter of the fine particles is within the above range, a filtration target can be brought into uniform contact with the inner surface of the pores in the porous membrane in a case where the filtration target is allowed to pass through the polyimide-based resin porous membrane obtained by removing the fine particles, and thus foreign substances contained in the filtration target can be efficiently adsorbed.

The particle diameter distribution index (d25/d75) of the fine particles is preferably in a range of 1 to 6 and more preferably in a range of 1.6 to 5, and still more preferably in a range of 2 to 4.

In a case where the particle diameter distribution index is set to be greater than or equal to the lower limits of the above-described preferable ranges, the porous membrane can be efficiently filled with fine particles, and thus a flow path is easily formed and the flow rate is improved. In addition, pores having different sizes are easily formed, different convections are generated, and thus the adsorption rate of foreign substances is further improved.

It is noted that d25 and d75 are values of particle diameters in which the cumulative frequencies of the particle diameter distribution are each 25% and 75%, and d25 is the value having the larger particle diameter in the present specification.

Further, in a case of forming an unsintered composite membrane in a two-layer shape in [Membrane formation of unsintered composite membrane] described below, as a fine particle (B1) that is used for a first varnish and a fine particle (B2) that is used for a second varnish, the same one may be used or those different from each other may be used. In order to make the pores on the side in contact with the base material denser, it is preferable that the fine particle (B1) has a small or the same particle diameter distribution index as compared with the fine particle (B2). Alternatively, it is preferable that the fine particle (B1) has a small or the same true sphere ratio as compared with the fine particle (B2). In addition, the fine particle (B11) preferably has a smaller particle diameter (average diameter) than the fine particle (B2), and in particular, it is preferable that the fine particle (B1) in a range of 100 to 1,000 nm (more preferably in a range of 100 to 600 nm) and the fine particle (B2) in a range of 500 to 2,000 nm (more preferably in a range of 700 to 2,000 nm) are each used. In a case where fine particle (B1) having a particle diameter smaller than that of the fine particles (B2) is used, the opening proportion of the pores on the surface of the polyimide-based resin porous membrane to be obtained can be increased, and the diameters thereof can be made uniform, and the strength of the porous membrane can be increased as compared with a case where the entire polyimide-based resin porous membrane is made of the fine particle (B1) alone.

In the present embodiment, a dispersant may be further added to the varnish together with the fine particles for the intended purpose of uniformly dispersing the fine particles. In a case of further adding a dispersing agent, it is possible to more uniformly mix polyamic acid, or polyimide or polyamide-imide with fine particles, and thus it is possible to more uniformly distribute the fine particles in the unsintered composite membrane. As a result of the above, it is possible to efficiently form a communication pore that is allowed to be connected to the front and back surfaces of the polyimide-based resin porous membrane, so that dense openings are provided on the surface of the polyimide-based resin porous membrane to be finally obtained and the air permeability of the polyimide-based resin porous membrane is improved.

As the dispersing agent, a known one can be used without particular limitation. Examples of the dispersing agent include anionic surfactants such as a palm fatty acid salt, a castor sulfate oil salt, a lauryl sulfate salt, a polyoxyalkylene allylphenyl ether sulfate salt, an alkylbenzene sulfonic acid, an alkylbenzene sulfonic acid salt, an alkyldiphenyl ether disulfonic acid salt, an alkylnaphthalene sulfonic acid salt, a dialkyl sulfosuccinate salt, isopropyl phosphate, a polyoxyethylene alkyl ether phosphate salt, and a polyoxyethylene allylphenyl ether phosphate salt; cationic surfactants such as an oleylamine acetic acid salt, laurylpyridinium chloride, cetylpyridinium chloride, lauryltrimethylammonium chloride, stearyltrimethylammonium chloride, behenyltrimethylammonium chloride, and didecyldimethylammonium chloride; amphoteric surfactants such as a palm alkyldimethylamine oxide, a fatty acid amide propyldimethylamine oxide, an alkylpolyaminoethylglycine hydrochloric acid salt, an amide betaine type activator, an alanine type activator, and lauryliminodipropionic acid; polyoxyalkylene primary alkyl ether-based or polyoxyalkylene secondary alkyl ether-based nonionic surfactants such as a polyoxyethylene octyl ether, a polyoxyethylene decyl ether, a polyoxyethylene lauryl ether, a polyoxyethylene laurylamine, a polyoxyethylene oleylamine, a polyoxyethylene polystyrylphenyl ether, and a polyoxyalkylene polystyrylphenyl ether, and polyoxyalkylene-based nonionic surfactants such as a polyoxyethylene dilaurate, a polyoxyethylene laurate, a polyoxyethylene hydrogenated castor oil, a polyoxyethylene hydrogenated castor oil, a sorbitan lauric acid ester, a polyoxyethylene sorbitan lauric acid ester, and a fatty acid diethanol amide; fatty acid alkyl esters such as octyl stearate and trimethylolpropane tridecanoate; and polyether polyols such as a polyoxyalkylene butyl ether, a polyoxyalkylene oleyl ether, and trimethylolpropane tris(polyoxyalkylene) ether. The above dispersing agent can be used alone, or two or more thereof can be mixed and used.

### Polyamic Acid

Examples of the polyamic acid that can be used in the present embodiment include those obtained by polymerizing any tetracarboxylic acid dianhydride with a diamine.

### Tetracarboxylic Acid Dianhydride

The tetracarboxylic acid dianhydride can be appropriately selected from the tetracarboxylic acid dianhydrides that are used as raw materials for synthesizing polyamic acids in the related art.

The tetracarboxylic acid dianhydride may be an aromatic tetracarboxylic acid dianhydride or may be an aliphatic tetracarboxylic acid dianhydride.

Examples of the aromatic tetracarboxylic acid dianhydride include pyromellitic acid dianhydride, 1,1-bis(2,3-dicarboxyphenyl)ethane dianhydride, bis(2,3-dicarboxyphenyl)methane dianhydride, bis(3,4-dicarboxyphenyl)methane dianhydride, 3,3′,4,4′-biphenyltetracarboxylic acid dianhydride, 2,3,3′,4′-biphenyltetracarboxylic acid dianhydride, 2,2,6,6-biphenyltetracarboxylic acid dianhydride, 2,2-bis(3,4-dicarboxyphenyl)propane dianhydride, 2,2-bis(2,3-dicarboxyphenyl)propane dianhydride, 2,2-bis(3,4-dicarboxyphenyl)-1,1,1,3,3,3-hexafluoropropane dianhydride, 2,2-bis(2,3-dicarboxyphenyl)-1,1,1,3,3,3-hexafluoropropane dianhydride, 3,3′,4,4′-benzophenonetetracarboxylic acid dianhydride, bis(3,4-dicarboxyphenyl)ether dianhydride, bis(2,3-dicarboxyphenyl)ether dianhydride, 2,2′,3,3′-benzophenonetetracarboxylic acid dianhydride, 4,4-(p-phenylenedioxy)diphthalic acid dianhydride, 4,4-(m-phenylenedioxy)diphthalic acid dianhydride, 1,2,5,6-naphthalenetetracarboxylic acid dianhydride, 1,4,5,8-naphthalenetetracarboxylic acid dianhydride, 2,3,6,7-naphthalenetetracarboxylic acid dianhydride, 1,2,3,4-benzenetetracarboxylic acid dianhydride, 3,4,9,10-perylenetetracarboxylic acid dianhydride, 2,3,6,7-anthracenetetracarboxylic acid dianhydride, 1,2,7,8-phenanthrenetetracarboxylic acid dianhydride, 9,9-bis phthalic acid anhydride fluorene, and 3,3′,4,4′-diphenylsulfonetetracarboxylic acid dianhydride.

Examples of the aliphatic tetracarboxylic acid dianhydride include ethylenetetracarboxylic acid dianhydride, butanetetracarboxylic acid dianhydride, cyclopentanetetracarboxylic acid dianhydride, cyclohexanetetracarboxylic acid dianhydride, 1,2,4,5-cyclohexanetetracarboxylic acid dianhydride, and 1,2,3,4-cyclohexanetetracarboxylic acid dianhydride.

Among the above, an aromatic tetracarboxylic acid dianhydride is preferable from the viewpoint of heat resistance of the polyimide resin to be obtained. Among them, 3,3′,4,4′-biphenyltetracarboxylic acid dianhydride or pyromellitic acid dianhydride is preferable from the viewpoint of price, availability.

The tetracarboxylic acid dianhydride may be used alone, or two or more thereof can be mixed and used.

### Diamine

A diamine can be appropriately selected from the diamines that are used as raw materials for synthesizing polyamic acids in the related art. This diamine may be an aromatic diamine or an aliphatic diamine; however, an aromatic diamine is preferable from the viewpoint of heat resistance of the polyimide resin to be obtained. The diamine can be used alone or in the form of a mixture of two or more kinds thereof.

Examples of the aromatic diamine include a diamino compound obtained by bonding one phenyl group or about 2 to 10 phenyl groups. Specific examples of the aromatic diamine include a phenylenediamine or a derivative thereof, a diaminobiphenyl compound or a derivative thereof, a diaminodiphenyl compound or a derivative thereof, a diaminotriphenyl compound or a derivative thereof, a diaminonaphthalene or a derivative thereof, an aminophenyl aminoindane or a derivative thereof, a diaminotetraphenyl compound or a derivative thereof, a diaminohexaphenyl compound or a derivative thereof, and a cardo type fluorinamine derivative.

The phenylene diamine is preferably m-phenylene diamine or p-phenylene diamine. Examples of the phenylene diamine derivative include diamines to which an alkyl group such as a methyl group or an ethyl group is bonded, for example, 2,4-diaminotoluene and 2,4-triphenylenediamine.

The diaminobiphenyl compound is a compound in which two aminophenyl groups are bonded to each other. Examples of the diaminobiphenyl compound include 4,4′-diaminobiphenyl, and 4,4′-diamino-2,2′-bis(trifluoromethyl)biphenyl.

The diaminodiphenyl compound is a compound obtained by bonding phenyl groups of two aminophenyl groups to each other through another group. Examples of the other group include an ether bond, a sulfonyl bond, a thioether bond, an alkylene group or a derivative group thereof, an imino bond, an azo bond, a phosphine oxide bond, an amide bond, and a ureylene bond. The alkylene group preferably has about 1 to 6 carbon atoms, and the derivative group thereof is a group in which one or more hydrogen atoms of an alkylene group are substituted with a halogen atom or the like.

Examples of the diaminodiphenyl compound include 3,3′-diaminodiphenyl ether, 3,4′-diaminodiphenyl ether, 4,4′-diaminodiphenyl ether, 3,3′-diaminodiphenyl sulfone, 3,4′-diaminodiphenyl sulfone, 4, 4′-diaminodiphenylsulfone, 3,3′-diaminodiphenylmethane, 3,4′-diaminodiphenylmethane, 4,4′-diaminodiphenylmethane, 4,4′-diaminodiphenylsulfide, 3,3′-diaminodiphenylketone, 3,4′-diaminodiphenylketone, 2,2-bis(p-aminophenyl)propane, 2,2′-bis(p-aminophenyl)hexafluoropropane, 4-methyl-2,4-bis(p-aminophenyl)-1-pentene, 4-methyl-2,4-bis(p-aminophenyl)-2-pentene, iminodianiline, 4-methyl-2,4-bis(p-aminophenyl)pentane, bis(p-aminophenyl)phosphine oxide, 4,4′-diaminoazobenzene, 4,4′-diaminodiphenylurea, 4,4′-diaminodiphenylamide, 1,4-bis(4-aminophenoxy)benzene, 1,3-bis(4-aminophenoxy)benzene, 1,3-bis(3-aminophenoxy)benzene, 4,4′-bis(4-aminophenoxy)biphenyl, bis[4-(4-aminophenoxy)phenyl]sulphone, bis[4-(3-aminophenoxy)phenyl]sulfone, 2,2-bis[4-(4-aminophenoxy)phenyl]propane, and 2,2-bis[4-(4-aminophenoxy)phenyl]hexafluoropropane.

The diaminotriphenyl compound is a compound obtained by bonding each of two aminophenyl groups and one phenylene group through another group. Examples of the other group include the same one as the other group in diaminodiphenyl compound. Examples of the diaminotriphenyl compound include 1,3-bis(m-aminophenoxy)benzene, 1,3-bis(p-aminophenoxy)benzene, and 1,4-bis(p-aminophenoxy)benzene.

Examples of diaminonaphthalene include 1,5-diaminonaphthalene and 2,6-diaminonaphthalene.

Examples of aminophenyl aminoindane include 5- or 6-amino-1-(p-aminophenyl)-1,3,3-trimethylindane.

Examples of the diaminotetraphenyl compound include 4,4′-bis(p-aminophenoxy)biphenyl, 2,2′-bis[p-(p′-aminophenoxy)phenyl]propane, 2,2′-bis[p-(p′-aminophenoxy)biphenyl]propane, and 2,2′-bis[p-(m-aminophenoxy)phenyl]benzophenone.

Examples of the cardo type fluorene amine derivative include 9,9-bisaniline fluorene.

The aliphatic diamine preferably has, for example, about 2 to 15 carbon atoms, and specific examples thereof include pentamethylenediamine, hexamethylenediamine, and heptamethylenediamine.

Here, the diamine may be a compound in which a hydrogen atom is substituted with at least one substituent selected from the group consisting of a halogen atom, a methyl group, a methoxy group, a cyano group, and a phenyl group.

Among the above, the diamine is preferably a phenylenediamine, a phenylenediamine derivative, or a diaminodiphenyl compound. Among them, p-phenylene diamine, m-phenylene diamine, 2,4-diaminotoluene, or 4,4′-diaminodiphenyl ether is particularly preferable from the viewpoints of the cost and the availability.

The production method for a polyamic acid is not particularly limited, and a known technique such as a method of reacting any tetracarboxylic acid dianhydride with a diamine in an organic solvent is used.

The reaction of tetracarboxylic acid dianhydride with a diamine is generally carried out in an organic solvent. The organic solvent used here is not particularly limited as long as it can dissolve each of tetracarboxylic acid dianhydride and a diamine and does not react with tetracarboxylic acid dianhydride and a diamine. The organic solvent can be used alone or in the form of a mixture of two or more kinds thereof.

Examples of the organic solvent that is used for the reaction between tetracarboxylic acid dianhydride and a diamine include nitrogen-containing polar solvents such as N-methyl-2-pyrrolidone, N,N-dimethylacetamide, N,N-diethylacetamide, N,N-dimethylformamide, N,N-diethylformamide, N-methylcaprolactam, N,N,N′,N′-tetramethyl urea; lactone-based polar solvents such as β-propiolactone, γ-butyrolactone, γ-valerolactone, δ-valerolactone, γ-caprolactone, and ε-caprolactone; dimethylsulfoxide; acetonitrile; fatty acid esters such as ethyl lactate and butyl lactate; ethers such as diethylene glycol dimethyl ether, diethylene glycol diethyl ether, dioxane, tetrahydrofuran, methyl cellosolve acetate, and ethyl cellesolve acetate; and phenol-based solvents such as cresols.

Among these, as the organic solvent here, it is preferable to use a nitrogen-containing polar solvent from the viewpoint of the solubility of the polyamic acid to be generated.

Further, from the viewpoint of membrane formation property and the like, it is preferable to use a mixed solvent containing a lactone-based polar solvent. In this case, the content of the lactone-based polar solvent is preferably in a range of 1% to 20% by mass and more preferably in a range of 5% to 15% by mass with respect to the entire organic solvent (100% by mass).

As the organic solvent here, it is preferable to use one or more selected from the group consisting of a nitrogen-containing polar solvent and a lactone-based polar solvent, and it is more preferable to use a mixed solvent of a nitrogen-containing polar solvent and a lactone-based polar solvent.

The using amount of the organic solvent is not particularly limited; however, it is preferably such an amount that the content of the generated polyamic acid in the reaction solution after the reaction is in a range of 5% to 50% by mass.

The using amount of each of tetracarboxylic acid dianhydride and a diamine used is not particularly limited; however, it is preferable to use an amount in a range of 0.50 to 1.50 mol, more preferable to use an amount in a range of 0.60 to 1.30 mol, and particularly preferable to use an amount in a range of 0.70 to 1.20 mol, with respect to the 1 mol of tetracarboxylic acid dianhydride.

The reaction (polymerization) temperature is generally in a range of −10° C. to 120° C. and preferably in a range of 5° C. to 30° C. The reaction (polymerization) time varies depending on the raw material composition to be used; however, it is generally in a range of 3 to 24 (hours).

The intrinsic viscosity of the polyamic acid solution obtained under such conditions is preferably in a range of 1,000 to 100,000 centipores (cP) (1 to 100 Pa·s) and more preferably 5,000 to 70,000 cP (5 to 70 Pa·s).

The intrinsic viscosity of the polyamic acid solution can be measured with an E type rotational viscometer under a temperature condition of 25° C.

### Polyimide

As the polyimide that can be used in the present embodiment, any known polyimide can be used as long as it can be dissolved in an organic solvent that is used for the varnish, without being limited by the structure and the molecular weight thereof.

The polyimide may have, in the side chain, a condensable functional group such as a carboxy group or a functional group that promotes a crosslinking reaction or the like during sintering.

In order to obtain polyimide that is soluble in an organic solvent that is used for the varnish, it is effective to use a monomer for introducing a flexible bent structure into the main chain.

Examples of this monomer include aliphatic diamines such as ethylenediamine, hexamethylenediamine, 1,4-diaminocyclohexane, 1,3-diaminocyclohexane, and 4,4′-diaminodicyclohexylmethane; aromatic diamines such as 2-methyl-1,4-phenylene, o-tolidine, m-tolidine, 3,3′-dimethoxybenzidine, and 4,4′-diaminobenzanilide; polyoxyalkylenediamines such as polyoxyethylenediamine, polyoxypropylenediamine, and polyoxybutylenediamine; polysiloxanediamine; 2,3,3′,4′-oxydiphthalic acid anhydride, 3,4,3′,4′-oxydiphthalic acid anhydride, and 2,2-bis(4-hydroxyphenyl)propanedibenzoate-3,3′,4,4′-tetracarboxylic acid dianhydride.

In addition, it is also effective to use a monomer having a functional group that improves the solubility in such an organic solvent. Examples of the monomer having such a functional group include fluorinated diamines such as 2,2′-bis(trifluoromethyl)-4,4′-diaminobiphenyl and 2-trifluoromethyl-1,4-phenylenediamine.

Further, in addition to the monomer having such a functional group, the monomer exemplified in the above description of the polyamic acid can be used in combination as long as the solubility is not impaired.

The production method for polyimide is not particularly limited, and examples thereof include known technique such as a method in which polyamic acid is chemically imidized or thermally imidized to be dissolved in an organic solvent.

Examples of the polyimide that can be used in the present embodiment include an aliphatic polyimide (a full-aliphatic polyimide) and an aromatic polyimide, and among them, an aromatic polyimide is preferable.

The aromatic polyimide may be one obtained by subjecting a polyamic acid having a constitutional unit represented by General Formula (5) to thermal or chemical ring-closure reaction or may be one obtained by dissolving a polyimide having a constitutional unit represented by General Formula (6) in a solvent.

In the formulae, R_{Ar} represents an aryl group, and R′_{Ar} represents an arylene group.

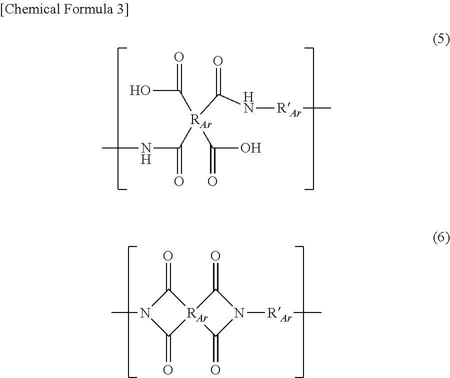

In the formulae, RA is not particularly limited as long as RA represents a cyclic conjugated system having (4n+2) π electrons, and may be monocyclic or polycyclic. The number of carbon atoms in the aromatic ring is preferably in a range of 5 to 30, more preferably in a range of 5 to 20, still more preferably in a range of 6 to 15, and particularly preferably in a range of 6 to 12. Specifically, as the aromatic ring, an aromatic hydrocarbon ring such as benzene, naphthalene, anthracene, and phenanthrene; and an aromatic heterocyclic ring in which some carbon atoms constituting the aromatic hydrocarbon ring have been substituted with heteroatoms are exemplary examples. Examples of the heteroatom in the aromatic heterocyclic rings include an oxygen atom, a sulfur atom, and a nitrogen atom. Specific examples of the aromatic heterocyclic ring include a pyridine ring and a thiophene ring. Among these, R_{Ar} represents preferably an aromatic hydrocarbon ring, more preferably benzene or naphthalene, and particularly preferably benzene.

In the formulae, examples of R′_{Ar} include a group obtained by removing two hydrogen atoms from the aromatic ring as R_{Ar}. Among these, R′_{Ar} represents preferably a group obtained by removing two hydrogen atoms from an aromatic hydrocarbon ring, more preferably a group obtained by removing two hydrogen atoms from benzene or naphthalene, and particularly preferably a phenylene group obtained by removing two hydrogen atoms from benzene.

The aryl group as RA and the arylene group as R′_{Ar} may each have a substituent.

### Polyamide-Imide

As the polyamide-imide that can be used in the present embodiment, any known polyimide can be used as long as it can be dissolved in an organic solvent that is used for the varnish, without being limited by the structure and the molecular weight thereof.

The polyamide-imide may have, in the side chain, a condensable functional group such as a carboxy group or a functional group that promotes a crosslinking reaction or the like during sintering.

As such a polyamide-imide, it is possible to use, without particular limitation, one obtained by reacting any trimellitic acid anhydride with a diisocyanate or one obtained by imidizing a precursor polymer that is obtained by reacting a reactive derivative of any trimellitic acid anhydride with a diamine.

Examples of the reactive derivative of any trimellitic acid anhydride include a halogenated trimellitic acid anhydride such as trimellitic anhydride chloride and a trimellitic acid anhydride ester.

Examples of any diisocyanate include metaphenylene diisocyanate, p-phenylene diisocyanate, o-tolidine diisocyanate, p-phenylene diisocyanate, m-phenylene diisocyanate, 4,4′-oxybis(phenyl isocyanate), 4,4′-diisocyanate diphenylmethane, bis[4-(4-isocyanoxidephenoxy)phenyl]sulfone, 2,2′-bis[4-(4-isocyanoxidephenoxy)phenyl]propane, 2,4-tolylene diisocyanate, 2,6-tolylene diisocyanate, 4,4′-diphenylmethane diisocyanate, 3,3′-dimethyldiphenyl-4,4′-diisocyanate, 3,3′-diethyldiphenyl-4,4′-diisocyanate, isophorone diisocyanate, hexamethylene diisocyanate, 4,4′-dicyclohexylmethane diisocyanate, m-xylene diisocyanate, p-xylene diisocyanate, and naphthalene diisocyanate.

Examples of any diamine include the same ones as the diamines exemplified in the description of the polyamic acid described above.

### Organic Solvent

The organic solvent that can be used for preparing the varnish is not particularly limited as long as it can dissolve the polyamic acid and/or the polyimide-based resin and does not dissolve fine particles, and examples thereof include the same one as the organic solvent that is used in the reaction between tetracarboxylic acid dianhydride and a diamine. The organic solvent can be used alone or in the form of a mixture of two or more kinds thereof.

The content of the organic solvent in the varnish is preferably in a range of 50% to 95% by mass and more preferably in a range of 60% to 85% by mass. The solid content concentration in the varnish is preferably in a range of 5% to 50% by mass and more preferably in a range of 15% to 40% by mass.

Further, in a case of forming an unsintered composite membrane in a two-layer shape in the section described in [Membrane formation of unsintered composite membrane] below, the volume fraction of polyamic acid or polyimide, or a polyamide-imide (B1) to a fine particle (B1) in the first varnish is preferably set in a range of 19:81 to 45:55. In a case where the total volume is assumed to be 100, particles are uniformly dispersed in a case where the volume occupied by the fine particle (B1) is 55 or greater, and particles are easily dispersed without being aggregated in a case where it is 81 or less. This makes it possible to uniformly form pores on the surface side of the base material of the polyimide-based resin molded membrane.

Further, the volume fraction of polyamic acid or polyimide, or a polyamide-imide (A2) to a fine particle (B2) in the second varnish is preferably set in a range of 20:80 to 50:50. In a case where the total volume is assumed to be 100, particles are easily dispersed singly and uniformly in a case where the volume occupied by the fine particle (B2) is 50 or greater, and particles not aggregated and surface cracking or the like hardly occurs in a case where it is 80 or less. As a result, a polyimide-based resin porous membrane having good mechanical characteristics such as stress and breaking elongation is easily formed.

Regarding the above-described volume fraction, it is preferable that the second varnish has a lower fine particle content rate than the first varnish. In a case where the above conditions are satisfied, the strength and flexibility of the unsintered composite membrane, the polyimide-based resin-fine particle composite membrane, and the polyimide-based resin porous membrane are ensured even in a case where polyamic acid or polyimide, or polyamide-imide is filled with fine particles in a high degree. Further, in a case where a layer having a low fine particle content rate is provided, the production cost can be reduced.

In a case of preparing a varnish, in addition to the above-described components, it is possible to blend, as necessary, known components such as an antistatic agent, a flame retardant, a chemical imidizing agent, a condensing agent, a mold releasing agent, and a surface conditioner for the intended purpose of prevention of static charge, flame retardancy impartment, low temperature sintering, mold releasability, coatability.

### [Membrane Formation of Unsintered Composite Membrane]

The membrane formation of the unsintered composite membrane containing polyamic acid or polyimide, or polyamide-imide and containing fine particles is carried out, for example, by coating a base material with the above varnish and drying under the conditions of normal pressure or vacuum at a temperature in a range of 0° C. to 120° C. (preferably 0° C. to 100° C.) and more preferably under the conditions of normal pressure at a temperature in a range of 60° C. to 95° C. (still more preferably 65° C. to 90° C.). The coating film thickness is, for example, preferably in a range of 1 to 500 μm and more preferably in a range of 5 to 50 μm.

Here, a mold releasing layer may be provided on the base material as necessary. Further, in the membrane formation of the unsintered composite membrane, each of a dipping step of carrying out dipping in a solvent containing water, a drying step, and a pressing step may be provided as optional steps before [Sintering of unsintered composite membrane] described below.

The mold releasing layer can be prepared by applying a mold releasing agent on the base material and carrying out drying or baking. As the mold releasing agent used here, a known mold releasing agent such as an alkyl phosphate ammonium salt-based mold releasing agent, a fluorine-based mold releasing agent, or a silicone-based mold releasing agent can be used without particular limitation. In a case where the unsintered composite membrane after drying is peeled from the base material, a small amount of the mold releasing agent remains on the peeled surface of the unsintered composite membrane. Since the remaining mold releasing agent may affect the wettability of the surface of the polyimide-based resin porous membrane and the mixing of impurities, it is preferable to remove the remaining mold releasing agent.

As a result, it is preferable to wash the unsintered composite membrane peeled from the base material with an organic solvent or the like. Examples of the washing method include known technique such as a method of dipping an unsintered composite membrane in a washing liquid and then taking it out and a method of shower washing.

In order to dry the unsintered composite membrane after washing, for example, the unsintered composite membrane after washing is air-dried at room temperature or heated to a suitable set temperature in a constant temperature bath. In this case, for example, it is possible to adopt a method in which an end part of the unsintered composite membrane is fixed to a mold made of SUS or the like to prevent deformation.

On the other hand, in a case where the base material is used as it is without providing a mold releasing layer in the membrane formation of the unsintered composite membrane, the step of forming the mold releasing layer and the step of washing the unsintered composite membrane can be omitted.

In addition, in a case where the unsintered composite membrane is formed into a membrane in a two-layer shape, first, the formation of a first unsintered composite membrane having a membrane thickness in a range of 1 to 5 μm is carried out by directly coating a base material such as glass with the above varnish and then drying under the conditions of normal pressure or vacuum at a temperature in a range of 0° C. to 120° C. (preferably in a range of 0° C. to 90° C.) and more preferably under the conditions of normal pressure at a temperature in a range of 10° C. to 100° C. (still more preferably in a range of 10° C. to 90° C.).

Subsequently, the first unsintered composite membrane is coated with the above second varnish, and drying is carried out in the same manner under the conditions of a temperature in a range of 0° C. to 80° C. (preferably in a range of 0° C. to 50° C.) and more preferably under the conditions of normal pressure at a temperature in a range of 10° C. to 80° C. (still more preferably in a range of 10° C. to 30° C.) to form a second unsintered composite membrane having a membrane thickness in a range of 5 to 50 m, whereby unsintered composite membrane having a two-layer shape can be formed into a membrane.

### [Sintering of Unsintered Composite Membrane]

After [Membrane formation of unsintered composite membrane] described above, the unsintered composite membrane is subjected to heat treatment (sintering) to form a composite membrane (a polyimide-based resin-fine particle composite membrane) consisting of a polyimide-based resin and fine particles.

In a case where the varnish contains polyamic acid, it is preferable to complete the imidization by [Sintering of unsintered composite membrane] of the present step.

The temperature (the sintering temperature) of the heat treatment varies depending on the structure of the polyamic acid, or polyimide or polyamide-imide contained in the unsintered composite membrane and the presence or absence of the condensing agent; however, it is preferably in a range of 120° C. to 400° C. and more preferably in a range of 150° C. to 375° C.

It is not always necessary to clearly separate the operation from the drying in the previous step in order to perform sintering. For example, in a case of carrying out sintering at 375° C., it is possible to use a method in which the temperature is raised from room temperature to 375° C. in 3 hours and then held at 375° C. for 20 minutes, or a stepwise drying-thermal imidization method in which the temperature is gradually raised from room temperature to 375° C. in 50° C. increments (held for 20 minutes in each increment) and finally held at 375° C. for 20 minutes. At that time, a method in which an end part of the unsintered composite membrane is fixed to a mold made of SUS or the like may be adopted to prevent deformation.

The thickness of the polyimide-based resin-fine particle composite membrane after the heat treatment (sintering) is, for example, preferably 1 μm or greater, more preferably in a range of 5 to 500 m, and still more preferably in a range of 8 to 100 μm.

The thickness of the polyimide-based resin-fine particle composite membrane can be determined by measuring thicknesses of a plurality of positions using a micrometer and averaging the measured thicknesses.

The present step is an optional step. The present step may not be carried out, particularly in a case where polyimide or polyamide-imide is used for the varnish.

### [Fine Particle Removal]

After [Sintering of unsintered composite membrane] described above, the fine particles are removed from the polyimide-based resin-fine particle composite membrane, whereby a polyimide-based resin porous membrane is produced.

For example, in a case where silica is used as the fine particle, the polyimide-based resin-fine particle composite membrane is brought into contact with a low-concentration hydrogen fluoride (HF) water to dissolve and remove the silica, whereby a porous membrane is obtained. In addition, in a case where the fine particles are resin fine particles, the resin fine particles are decomposed and removed by heating to a temperature higher than or equal to the thermal decomposition temperature of the resin fine particles and lower than the thermal decomposition temperature of the polyimide-based resin, whereby a porous membrane is obtained.

### [Etching (Decyclization of Imide Bond)]

The etching step can be carried out by a chemical etching method or a physical removal method, or a method in which these methods are combined.

### In Regard to Chemical Etching Method

A known technique in the related art can be used as the chemical etching method.

The chemical etching method is not particularly limited, and examples thereof include a treatment with an etching liquid such as an inorganic alkaline solution or an organic alkaline solution. Among the above, a treatment with an inorganic alkaline solution is preferable.

Examples of the inorganic alkaline solution include a hydrazine solution containing hydrazine hydrate and ethylenediamine; a solution of an alkali metal hydroxide such as potassium hydroxide, sodium hydroxide, sodium carbonate, sodium silicate, or sodium metasilicate; an ammonia solution; and an etching liquid containing alkali hydroxide, hydrazine, and 1,3-dimethyl-2-imidazolidinone as main components.

Examples of the organic alkaline solution include primary amines such as ethyl amine and n-propyl amine; secondary amines such as diethyl amine and di-n-butyl amine; tertiary amines such as triethyl amine and methyldiethyl amine; alcohol amines such as dimethylethanol amine and triethanol amine; quaternary ammonium salts such as tetramethylammonium hydroxide and tetraethylammonium hydroxide; and alkaline etching liquids such as cyclic amines such as pyrrole and piperidine. The alkali concentration in the etching liquid is, for example, in a range of 0.01% to 20% by mass.

Pure water or alcohols can be appropriately selected as the solvent for each of the above etching liquids, and those to which a suitable amount of a surfactant is added can also be used.

### In Regard to Physical Removal Method

As the physical removal method, for example, a dry etching method using plasma (oxygen, argon, or the like), corona discharge, or the like can be used.

The above-described chemical etching method or physical removal method can be applied before [Removal of fine particles] described above or can be applied after [Removal of fine particles] described above.

Among the above, it is preferable to be applied after [Removal of fine particles] described above since the communication pores inside the polyimide-based resin porous membrane are more easily formed and the foreign substance removing property is enhanced.

In a case where the chemical etching method is carried out in the etching step, a step of washing the polyimide-based resin porous membrane may be provided after the present step in order to remove excess etching liquid.

The washing after the chemical etching may be carried out by washing with water alone; however, it is preferable to combine washing with acid and washing with water.

Further, after the etching step, the polyimide-based resin porous membrane may be subjected to heat treatment (re-sintering) in order to improve the wettability of the polyimide-based resin porous membrane surface to an organic solvent and remove residual organic substances. The conditions of this heating are the same as the conditions in [Sintering of unsintered composite membrane] described above.

For example, in the polyimide-based resin porous membrane produced by the above-described production method, spherical cells and a communication pore in which adjacent spherical cells communicate with each other are formed. The polyimide-based resin porous membrane preferably has a communication pore so that a communication pore that opens on one outer surface is connected to the inside of the porous membrane and opens up to the other (on the back side) outer surface, and thus a flow path in which a fluid can pass through the porous membrane is ensured.

The Garley air permeability of the “porous membrane in which adjacent spherical cells communicate with each other” is, for example, preferably 30 seconds or longer from the viewpoint of efficiently removing foreign substances while maintaining a certain degree of high flow rate of a filtration target that passes through the porous membrane. The Gurley air permeability of the porous membrane is more preferably in a range of 30 to 1,000 seconds, still more preferably in a range of 30 to 600 seconds, and particularly preferably in a range of 30 to 500 seconds. In a case where the Garley air permeability is less than or equal to the above-described preferable ranges, the degree of porosity (the presence ratio of the communication pore and the like) is sufficiently high, and thus the effect of removing foreign substances can be more easily obtained.

The Garley air permeability of the porous membrane can be measured according to JIS P 8117.

The “porous membrane in which adjacent spherical cells communicate with each other” has communication pores having an average pore diameter of preferably 1 to 1000 nm, more preferably 3 to 300 nm, and still more preferably 5 to 100 nm. The average pore diameter thereof is particularly preferably 10 to 80 nm.

The pore diameter of the communication pore means the diameter of the communication pore. Since one communication pore is usually formed of two adjacent particles by the above-described production method, the diameter may be a diameter in a direction perpendicular to a longitudinal direction in a case where the direction in which two pores constituting the communication pores are adjacent to each other is defined as the longitudinal direction, and thus the pore diameter of the communication pores tends to decrease in a case where the step of (decyclization of an imide bond) is not provided.

The average pore diameter of the communication pores formed by communication of the adjacent spherical cells in the porous membrane (also referred to as the average pore diameter of the porous membrane) is a value measured using a palm porometer (for example, manufactured by PMI) according to the half-dry method (ASTM E1294-89) using perfluoropolyester (trade name, Galwick, interfacial tension value: 15.9 dyne/cm) as the test liquid at a measurement temperature of 25° C. and a measurement pressure range of 0 to 400 psi.

In addition, the average pore diameter of the spherical cells of “porous membrane in which adjacent spherical cells communicate with each other” is preferably in a range of 10 to 2500 nm, more preferably in a range of 20 to 1000 nm, still more preferably in a range of 30 to 500 nm, and particularly preferably in a range of 40 to 400 nm. Regarding a porous membrane (for example, a porous polyamide-imide membrane) that is not subjected to chemical etching, the average particle diameter of the fine particles that are used in the production of the porous membrane is defined as the average pore diameter of the spherical cells.

As described above, “porous membrane in which adjacent spherical cells communicate with each other” is preferably a porous membrane containing pores having an average pore diameter of several hundred nanometers. Therefore, for example, even minute substances in the nanometer unit can be adsorbed or captured in the pores and/or the communication pores in the porous membrane.

Regarding the pore diameter of the communication pore, in a case where the distribution of the pore diameters of the individual pores that impart porosity to “porous membrane in which adjacent spherical cells communicate with each other” is broader, the pore diameter of the communication pore that is formed by pores adjacent to each other tends to decrease.

From the viewpoint of reducing the pore diameter of the communication pores, the void ratio of the “porous membrane in which adjacent spherical cells communicate with each other” is, for example, preferably 50% by mass or greater, more preferably in a range of 55% to 90% by mass, still more preferably in a range of 60% to 80% by mass, and particularly preferably in a range of about 60% to 70% by mass. In a case where the void ratio is greater than or equal to the above-described lower limits of the above-described ranges, the effect of removing foreign substances can be more easily obtained. In a case where the void ratio is less than or equal to the upper limits of the above-described ranges, the strength of the porous membrane is further increased.

The void ratio of the porous membrane is determined by calculating the proportion of the mass of the fine particles with respect to the total mass of the resin and the like that are used in the production of the porous membrane, and the fine particles.

The “porous membrane in which adjacent spherical cells communicate with each other” is excellent in mechanical characteristics such as stress and breaking elongation.

The stress of the “porous membrane in which adjacent spherical cells communicate with each other” provided in the filter is, for example, preferably 10 MPa or greater, more preferably 15 MPa or greater, and still more preferably in a range of 15 to 60 MPa.

The stress of the porous membrane is a value measured by preparing a sample having a size of 4 mm×30 mm and subjecting the sample to the measurement using a testing machine under the measuring condition of 5 mm/min.

Further, the breaking elongation of the “porous membrane in which adjacent spherical cells communicate with each other” is, for example, preferably 10% GL or greater and more preferably 15% GL or greater. The upper limit of the breaking elongation is, for example, preferably 60% GL or less and more preferably 55% GL or less. In a case where the void ratio of the polyimide-based resin porous membrane decreases, breaking elongation tends to increase.

The breaking elongation of the porous membrane is a value measured by preparing a sample having a size of 4 mm×30 mm and subjecting the sample to the measurement using a testing machine under the measuring condition of 5 mm/min.

The thermal decomposition temperature of “porous membrane in which adjacent spherical cells communicate with each other” is preferably 200° C. or higher, more preferably 320° C. or higher, and still more preferably 350° C. or higher.

The thermal decomposition temperature of the porous membrane can be measured by raising the temperature to 1,000° C. at a temperature rising rate of 10° C./min in an air atmosphere.

The filter in the present embodiment is not limited to the one that includes a porous membrane in which the communication pores 5 in which the adjacent spherical cell 1a and the spherical cell 1b communicate with each other as shown in FIG. 1 is formed, and may include a porous membrane in which, in addition to the communication pores 5, a cell or communication pore having another form is formed. Examples of the cell (hereinafter, referred to as “another cell”) having another form include a cell that differs in shape or pore diameter, and examples thereof include an elliptical cell, a polyhedral cell, a spherical cell having a different pore diameter. Examples of the above-described “communication pore having another form” include a communication pore in which a spherical cell and another cell communicate with each other.

The shape or pore diameter of another cell may be appropriately determined depending on the kinds of impurities to be removed. The communication pore in which a spherical cell and another cell communicate with each other can be formed by the selection of material of the fine particle material described above, the shape control of the fine particles, and the like.

According to the filter including a porous membrane in which, in addition to the communication pore in which adjacent spherical cells communicate with each other, a cell or communication pore having another form is formed, it is possible to efficiently remove various foreign substances from a filtration target.

Further, the filter in the present embodiment replaces a filter cartridge or the like for removing impurities having a fine particle shape, which has been installed in the related art, in the supply lines of various chemical liquids or the point of use (POU) in the semiconductor manufacturing process or can be used in combination with these. As a result, various foreign substances can be efficiently removed from a filtration target (a chemical liquid for lithography) using the same device and operation as those in the related art, and a high-purity resist composition purified product can be prepared.

### <<Filtration of Resist Composition>>

Filtration of a resist composition using a filter having a porous structure in which adjacent spherical cells communicate with each other may be carried out in a state where differential pressure is not provided (that is, a resist composition may be allowed to pass through the filter only by gravity) or may be carried out in a state where differential pressure is provided. Among the above, the latter is preferable, and it is preferable to carry out an operation of allowing a resist composition to pass through the filter by differential pressure.

The “state where differential pressure is provided” means that a pressure difference is present between one side and the other side of the polyimide-based resin porous membrane provided in the filter.

For example, pressurization (positive pressure) that applies pressure to one side (the resist composition supply side) of the polyimide-based resin porous membrane, and decompression (negative pressure) that causes one side (the filtrate side) of the polyimide-based resin porous membrane to be negative pressure. In the filtration step according to the present embodiment, the former pressurization is preferable.

The pressurization is an operation to apply pressure to the supply liquid side of the polyimide-based resin porous membrane, where a resist composition (hereinafter, may be referred to as a “supply liquid”) before being allowed to pass through the polyimide-based resin porous membrane is present on the supply liquid side. For example, it is preferable to apply pressure to the supply liquid side by using the liquid flow pressure generated by the circulation or liquid feeding of the supply liquid or by using the positive pressure of the gas.

The liquid flow pressure can be generated, for example, by an active liquid flow pressure applying method with a pump (a feeding pump, a circulation pump, or the like). Examples of the pump include a rotary pump, a diaphragm pump, a metering pump, a chemical pump, a plunger pump, a bellows pump, a gear pump, a vacuum pump, an air pump, and a liquid pump.

In a case where the supply liquid is circulated or fed by the pump, the pump is generally arranged between the supply liquid bath (or the circulation bath) and the polyimide-based resin porous membrane.

For example, in a case where a supply liquid is allowed to pass through the polyimide-based resin porous membrane only by gravity, the liquid flow pressure may be the pressure that is applied to the polyimide-based resin porous membrane by the supply liquid; however, it is preferably the pressure that is applied by the active liquid flow pressure applying method.

The gas that is used for pressurization is preferably a gas that is inert or non-reactive to the supply liquid, and specific examples thereof include nitrogen and a rare gas such as helium and argon.

Regarding a method of applying pressure to the supply liquid side, it is preferable to use the positive pressure of the gas. At that time, the filtrate side where the filtrate has passed through the polyimide-based resin porous membrane may be at atmospheric pressure without being decompressed.

Further, the pressurization may utilize both the liquid flow pressure and the positive pressure of the gas. Further, the differential pressure may be a combination of pressurization and decompression, and for example, one that utilizes both liquid flow pressure and decompression, one that utilizes both positive pressure and decompression of the gas, or one that utilizes liquid flow pressure, and positive pressure and decompression of the gas may be used. In a case where the method of providing differential pressure is combined, a combination of liquid flow pressure and positive pressure of the gas or a combination of liquid flow pressure and decompression is preferable from the viewpoint of simplification of production.

In the present embodiment, as the method of providing differential pressure, for example, even one method using positive pressure by gas or the like is excellent in foreign substance removing property since the polyimide-based resin porous membrane is used.

The decompression is an operation to decompress the filtrate side where the filtrate has passed through the polyimide-based resin porous membrane. For example, the decompression may be decompression by a pump; however, it is preferable to reduce pressure to form a vacuum.

In a case where the operation of allowing the resist composition to pass through the filter is carried out in a state where differential pressure is provided, the pressure difference is appropriately set in consideration of the membrane thickness, void ratio, or average pore diameter of the polyimide-based resin porous membrane to be used, or the desired degree of purification, the flow amount, the flow rate, or the concentration or viscosity of the supply liquid, or the like. For example, in a case of the so-called cross-flow method (the supply liquid is allowed to flow in parallel with the polyimide-based resin porous membrane), the pressure difference is preferably, for example, 0.3 MPa or less.

In a case of the so-called dead-end method (the supply liquid is allowed to flow to intersect the polyimide-based resin porous membrane), the pressure difference is, for example, preferably 1 MPa or less and more preferably 0.3 MPa or less. The lower limit of each of the pressure differences is preferably 0.01 MPa or greater and more preferably 0.05 MPa or greater.

In the step (i), the operation of allowing the resist composition to pass through the filter having the polyimide-based resin porous membrane described above can be carried out while maintaining a high flow rate of the resist composition (the supply liquid).

The flow rate in this case is not particularly limited; however, for example, the flow rate of pure water in a case of being pressurized at 0.08 MPa at room temperature (20° C.) is preferably 1 mL/min or greater, more preferably 3 mL/min or greater, still more preferably 5 mL/min or greater, and particularly preferably 10 mL/min or greater. The upper limit of the flow rate is not particularly limited, and is, for example, 50 mL/min or less.

In the present embodiment, since the filter having the polyimide-based resin porous membrane described above is used, filtration can be carried out while maintaining a high flow rate in this manner, and the removal rate of foreign substances contained in the resist composition can be increased.

In addition, in the step (i), the operation of allowing the resist composition to pass through the filter is preferably carried out by setting the temperature of the resist composition in a range of to 0° C. to 30° C. and preferably carried out by setting in a range of 5° C. to 25° C.

Further, in the step (i), the resist composition may be allowed to pass through the filter having the polyimide-based resin porous membrane a plurality of times (may be subjected recirculation filtration a plurality of times) or may be allowed to pass through a plurality of filters including at least a filter having the polyimide-based resin porous membrane.

### <Other Steps>

The production method according to the present embodiment may include other steps in addition to the step (i).

Examples of the other steps include a step of bringing an alcohol such as methanol, ethanol, or isopropyl alcohol, a ketone such as acetone or methyl ethyl ketone, water, a solvent contained in the supply liquid, or a solution of a mixture thereof into contact with the polyimide-based resin porous membrane and passing the liquid through the membrane for washing the polyimide-based resin porous membrane, improving wettability with respect to the supply liquid, or adjusting the surface energy between the polyimide-based resin porous membrane and the supply liquid before the supply liquid passes through the polyimide-based resin porous membrane.

In order to bring the above solution into contact with the polyimide-based resin porous membrane, the polyimide-based resin porous membrane may be impregnated or dipped in the above solution. In a case where the above solution is brought into contact with the polyimide-based resin porous membrane, for example, the solution can be permeated into the pores inside the polyimide-based resin porous membrane. The bringing of the solution into contact with the polyimide-based resin porous membrane may be carried out in a state where the differential pressure described above is provided and is preferably carried out under pressure particularly in a case where the solution is allowed to permeate into the pores inside the polyimide-based resin porous membrane.

<<Step (ii)>>

The production method according to the present embodiment may further include a step (ii) of washing the filter by bringing a membrane washing liquid into contact with the filter before the step (i).

The membrane washing liquid can be used without particular limitation as long as the membrane washing liquid can remove the organic residue or metal impurities adhering to the filter. Preferred examples of the membrane washing liquid include those containing a solvent and a metal removing agent.

The solvent in the membrane washing liquid can be appropriately selected from known organic solvents, and specific examples thereof include polar solvents such as a ketone-based solvent, an ester-based solvent, an alcohol-based solvent, a nitrile-based solvent, an amide-based solvent, an ether-based solvent, a sulfoxide-based solvent, and a sulfone-based solvent; and non-polar solvents such as a hydrocarbon-based solvent.

The organic solvent also includes an organic solvent that contains a plurality of kinds of functional groups characterizing each solvent in the structure thereof, and in such cases, the organic solvent is considered to correspond to any solvent kind that contains the functional groups of the organic solvent. For example, diethylene glycol monomethyl ether corresponds to both alcohol-based solvents and ether-based solvents in the above classification.

Among the examples, as the solvent in the membrane washing liquid, a solvent selected from the group consisting of a ketone-based solvent, an ester-based solvent, an alcohol-based solvent, and an ether-based solvent is preferable, and a mixed solvent of two or more solvents is more preferable. Further, the two or more kinds of solvents may contain at least two kinds of compounds and may be two or more kinds of solvents in the same classification.

Examples of the metal removing agent in the membrane washing liquid include metal chelating agents and organic acids.

Examples of the metal chelating agent include amino carboxylic acid-based chelating agents such as ethylenediaminetetraacetic acid, nitrilotriacetic acid, and diethylenetriaminepentaacetic acid; phosphonic acid-based chelating agents such as 1-hydroxyethane-1,1-diphosphonic acid and nitrilotris(methylenephosphonic acid); and a compound (a1) represented by General Formula (a-1). Among these, the compound (a1) is preferable as the metal chelating agent.

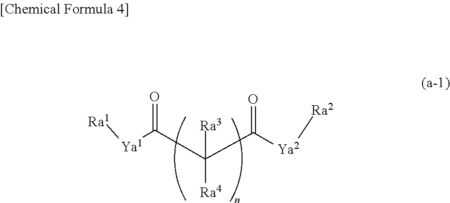

[In the formula, Ra¹ and Ra² each independently represent an alkyl group having 1 to 3 carbon atoms. Ra³ and Ra⁴ each independently represent a hydrogen atom or an alkyl group having 1 to 3 carbon atoms. Ya¹ and Ya² each independently represent a single bond, —O—, —S—, or —N(Ra⁵)—. Ra⁵ represents a hydrogen atom or an alkyl group having 1 to 3 carbon atoms. n represents an integer of 0 to 3.]

In Formula (a-1), examples of the alkyl group having 1 to 3 carbon atoms represented by Ra¹ to Ra⁵ include a methyl group, an ethyl group, a propyl group, and an isopropyl group.

In Formula (a-1), Ra¹ and Ra² each independently represent preferably a methyl group or an ethyl group and more preferably a methyl group.

It is preferable that Ra³ and Ra⁴ each represent a hydrogen atom.

Ya¹ represents preferably a single bond or —O— and more preferably a single bond.

Ya² represents preferably a single bond or —O— and more preferably a single bond. n represents preferably 1 or 2 and more preferably 1.

Among the examples, as the compound (a1), acetylacetone (AcAc) or acetonylacetone is preferable, and acetylacetone (AcAc) is more preferable.

In the membrane washing liquid, the metal chelating agent may be used alone or in combination of two or more kinds thereof.

Examples of the organic acid include a carboxylic acid such as lactic acid (LA), citric acid, malic acid, formic acid, acetic acid, oxalic acid, 2-nitrophenylacetic acid, 2-ethylhexanoic acid, or dodecanoic acid; saccharic acid such as ascorbic acid or glucuronic acid; sulfonic acid such as methanesulfonic acid, benzenesulfonic acid, or p-toluenesulfonic acid; and phosphoric acid ester and phosphoric acid such as bis(2-ethylhexyl) phosphoric acid.

Among these, the organic acid is preferably a carboxylic acid, more preferably hydroxy acid, still more preferably at least one selected from the group consisting of lactic acid, citric acid, and malic acid, and particularly preferably lactic acid.

In the membrane washing liquid, the organic acid may be used alone or in combination of two or more kinds thereof.

It is preferable that the membrane washing liquid contains two or more kinds of metal removing agents.

The membrane washing liquid contains preferably two or more kinds of metal removing agents selected from the group consisting of the above-described metal chelating agent and organic acid, more preferably two or more kinds of metal removing agents selected from the group consisting of the above-described compound (a1) and organic acid, and still more preferably two or more kinds of metal removing agents selected from the group consisting of the above-described compound (a1) and a carboxylic acid.

Among the examples, the membrane washing liquid contains preferably a combination of the above-described metal chelating agent and an organic acid, more preferably a combination of the above-described compound (a1) and an organic acid, and still more preferably a combination of the above-described compound (a1) and a carboxylic acid.

The membrane washing liquid may contain optional components other than the solvents and the metal removing agents described above. Examples of the optional components include pH adjusters and surfactants.

Preferred examples of the membrane washing liquid include a membrane washing liquid containing a solvent and a metal removing agent, in which the membrane washing liquid contains two or more kinds of solvents, and the distance between the Hansen solubility parameter of the membrane washing liquid and the Hansen solubility parameter of dimethylacetamide is 1.0 or less (see WO2022/107795A).

Examples of the operation of washing the filter by bringing the above-described membrane washing liquid into contact with the filter include a method of immersing the filter in the membrane washing liquid and a method of spraying the membrane washing liquid onto the filter. The washing operation may be performed once or plurality of times.

In addition, the membrane washing liquid may be heated during the washing operation, or the washing operation may be performed at room temperature (for example, 23° C.).

Examples of the other steps include a step of carrying out filtration with a filter other than the filter having a polyimide-based resin porous membrane. The filter other than the filter having a polyimide-based resin porous membrane is not particularly limited, and examples thereof include a filter having a porous membrane of a thermoplastic resin, such as a nylon membrane, a polyethylene membrane, a polypropylene membrane, a polytetrafluoroethylene (PTFE) membrane, a tetrafluoroethylene/perfluoroalkyl vinyl ether copolymer (PFA) membrane, or a membrane obtained by modifying these membranes. Among the above, as the other filter, a filter having a porous membrane containing a polyethylene resin is preferable since it is excellent in foreign substance removing property.

<<Step (iii)>>

The production method according to the present embodiment may further include a step (iii) of filtering the composition through a filter including a porous membrane containing a polyethylene resin, in addition to the step (i) or the step (i) and the step (ii).

A porous membrane (hereinafter, also referred to as a “polyethylene resin porous membrane”) containing a polyethylene resin may be composed of only a polyethylene resin or may contain a polyethylene resin and another resin; however, it is preferably composed of only a polyethylene resin.

The polyethylene resin porous membrane is not particularly limited, and a known one can be used. Since the polyethylene resin porous membrane is excellent in impact resistance, abrasion resistance, and chemical resistance, it is preferable to use a porous membrane of an ultrahigh molecular weight polyethylene (UPE).

The average pore diameter of such a polyethylene resin porous membrane is not particularly limited; however, it is preferably in a range of 0.1 to 100 nm, more preferably in a range of 0.3 to 50 nm, and still more preferably in a range of 0.5 to 10 nm, from the viewpoint of removing fine foreign substances.

Examples of such a filter having a polyethylene resin porous membrane include those in which a polyethylene resin porous membrane is provided in an outer container made of a thermoplastic resin (polyethylene, polypropylene, PFA, polyether sulfone (PES), polyimide, polyamide-imide, or the like).

The step (iii) is preferably carried out after the step (i), and in this case, the average pore diameter of the polyethylene resin porous membrane is preferably less than the average pore diameter of the communication pores of the polyimide-based porous membrane.

In the production method according to the present embodiment, the step (iii) may be repeated after the step (i). In this case, the resist composition (the supply liquid) is allowed to pass through a filter having a polyimide-based resin porous membrane and a filter having a polyethylene resin porous membrane, while being circulated at all times. In a case of performing circulation type filtration as described above, it is preferable that both filters are arranged so that the resist composition passes through the filter having a polyimide-based resin porous membrane and then passes through the filter having a polyethylene resin porous membrane in the circulation path.

In a case where the step (iii) is carried out, an alcohol such as methanol, ethanol, or isopropyl alcohol, a ketone such as acetone or methyl ethyl ketone, water, a solvent contained in the supply liquid, or a solution of a mixture thereof is brought into contact with the polyethylene resin porous membrane and allowed to pass through the membrane for washing the polyethylene resin porous membrane, improving wettability with respect to the supply liquid, or adjusting the surface energy between the polyethylene resin porous membrane and the supply liquid before the supply liquid passes through the polyethylene resin porous membrane similarly to the description in the section of the step (i).

### <Resist Composition>

The resist composition, which is the filtration target, generates an acid upon light exposure, and whose solubility in a developing solution is changed by the action of the acid.

Such a resist composition contains a base material component (A) (hereinafter, also referred to as “component (A)”) whose solubility in a developing solution is changed by the action of acid, and an organic solvent component (S) (hereinafter, also referred to as “component (S)”). In the resist composition, which is the filtration target in the present embodiment, the component (A) includes a resin component (A1) that generates an acid upon light exposure and whose solubility in a developing solution is changed by the action of the acid. The resist composition may contain other optional components, as necessary, in addition to the component (A1) and the component (S).

In a case where a resist film is formed using the resist composition and the formed resist film is subjected to selective exposure, an acid is generated from the component (A) at exposed portions of the resist film, and the generated acid acts on the component (A) to change the solubility of the component (A) in a developing solution, whereas the solubility of the component (A) in a developing solution is not changed at unexposed portions of the resist film, and thus a difference in solubility in the developing solution between exposed portions and unexposed portions of the resist film is generated. Therefore, in a case where the resist film is developed, the exposed portion of the resist film is dissolved and removed to form a positive-tone resist pattern in a case where the resist composition is of a positive tone, whereas the unexposed portion of the resist film is dissolved and removed to form a negative tone resist pattern in a case where the resist composition is of a negative tone.

The resist composition, which is the filtration target, may be a positive-tone resist composition or a negative-tone resist composition. Further, in the formation of a resist pattern, the resist composition may be applied to an alkali developing process using an alkali developing solution in the developing treatment, or a solvent developing process using a developing solution (an organic developing solution) containing an organic solvent in the developing treatment.

### <<Base Material Component (A)>>

In the resist composition, which is the filtration target, the component (A) includes a resin component (A1) (hereinafter, also referred to as “component (A1)”) whose solubility in a developing solution is changed by the action of the acid.

Since the polarity of the base material component before and after the light exposure is changed by using the component (A1), an excellent development contrast can be obtained not only in an alkali developing process but also in a solvent developing process.

As the component (A), at least one of other polymer compounds or low-molecular-weight compounds may be used in combination with the component (A1).

In the resist composition, the component (A) may be used alone or in a combination of two or more kinds thereof.

### In Regard to Resin Component (A1)

The component (A1) is a resin component whose solubility in a developing solution is changed by the action of an acid. The component (A1) includes a copolymer having a constitutional unit (a01) with an onium salt structure that generates sulfonic acid upon light exposure and a constitutional unit (a02) with an onium salt structure that generates a carboxylic acid upon light exposure.

Both the constitutional unit (a01) and the constitutional unit (a02) are constitutional units that generate an acid upon light exposure. In the example of the resist composition, the constitutional unit (a02) includes a form in which an acid generated from the constitutional unit (a01) upon light exposure is trapped (the diffusion of the acid is controlled).

The component (A1) may have other constitutional units as necessary in addition to the constitutional unit (a01) and the constitutional unit (a02).

<<Constitutional Unit (a01)>>

The constitutional unit (a01) is a constitutional unit having an onium salt structure that generates sulfonic acid upon light exposure.

Examples of the constitutional unit (a01) include a constitutional unit (anion-bound unit) having an anion group (—SO₃⁻) which generates sulfonic acid upon light exposure in a side chain and a constitutional unit (cation-bound unit) having a cation group (onium cation) which is decomposed upon light exposure in a side chain.

Among these, in the formation of the resist pattern, from the viewpoint that satisfactory lithography characteristics are likely to be obtained, it is preferable that the constitutional unit (a01) is a constitutional unit (anion-bound unit) having an anion group (—SO₃⁻) which generates sulfonic acid upon light exposure in a side chain.

Suitable examples of the constitutional unit (a01) include a constitutional unit derived from a compound represented by General Formula (a01-b).

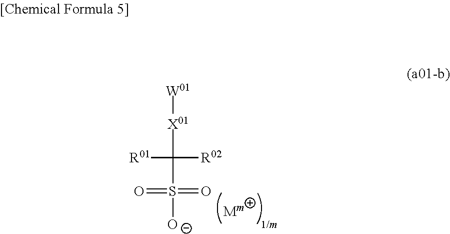

[In Formula (a01-b), W⁰¹ represents a polymerizable group-containing group. X⁰¹ represents a divalent linking group or a single bond. R⁰¹ and R⁰² each independently represent a fluorinated alkyl group, a fluorine atom, or a hydrogen atom. M^{m+} represents an m-valent onium cation. m represents an integer of 1 or greater.]

### {Anion Moiety}

In Formula (a01-b), the term “polymerizable group” in the polymerizable group-containing group as W⁰¹ is a group that enables polymerization of a compound containing a polymerizable group by radical polymerization or the like and, for example, is a group having a carbon-carbon multiple bond between carbon atoms such as an ethylenic double bond.

Examples of the polymerizable group include a vinyl group, an α-methylvinyl group, an allyl group, an acryloyl group, a methacryloyl group, a fluorovinyl group, a difluorovinyl group, a trifluorovinyl group, a difluorotrifluoromethylvinyl group, a trifluoroallyl group, a perfluoroallyl group, a trifluoromethylacryloyl group, a nonylfluorobutylacryloyl group, a vinyl ether group, a fluorine-containing vinyl ether group, an allyl ether group, a fluorine-containing allyl ether group, a styryl group, a vinylnaphthyl group, a fluorine-containing styryl group, a fluorine-containing vinylnaphthyl group, a norbornyl group, a fluorine-containing norbornyl group, a silyl group, and a maleimide group.

The polymerizable group-containing group may be a group formed of only a polymerizable group, or a group formed of a polymerizable group and a group other than the polymerizable group. Examples of the group other than the polymerizable group include a divalent hydrocarbon group which may have a substituent and a divalent linking group containing a heteroatom.

Suitable examples of the polymerizable group-containing group include a group represented by Formula: C(R^{X11})(R^{X12})═C(R^{X13})-Ya^{x0}-. In the formula, R^{X11}, R^{X12}, and R^{X13} each independently represent an alkyl group having 1 to 5 carbon atoms, a halogenated alkyl group having 1 to 5 carbon atoms, a halogen atom, or a hydrogen atom, and Ya^{x0} represents a divalent linking group or a single bond.

The alkyl group having 1 to 5 carbon atoms as R^{X11}, R^{X12}, and R^{X13} is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms are substituted with halogen atoms. As the halogen atom, a fluorine atom is particularly preferable. It is preferable that R^{X11}, R^{X12}, and R^{X13} represent a hydrogen atom, an alkyl group having 1 to 5 carbon atoms, or a fluorinated alkyl group having 1 to 5 carbon atoms and particularly preferable that R^{X11} and R^{X12} represent a hydrogen atom and R^{X13} represents a hydrogen atom or a methyl group from the viewpoint of industrial availability.

Examples of the divalent linking group as Ya^{x0} include a divalent hydrocarbon group which may have a substituent and a divalent linking group having a heteroatom. Examples of the divalent linking group as Ya^{x0} include the same groups as described in the section of the divalent linking group as X⁰¹ in Formula (a01-b), and suitable examples thereof include an ester bond (—C(═O)—O—), an oxycarbonyl group (—O—C(═O)—), an ether bond (—O—), a linear or branched alkylene group, an arylene group which may have a substituent, and optional combinations thereof.

In Formula (a01-b), examples of the divalent linking group as X⁰¹ include a divalent hydrocarbon group which may have a substituent and a divalent linking group having a heteroatom.

Divalent Hydrocarbon Group which May have Substituent:

The divalent hydrocarbon group which may have a substituent as X⁰¹ may be an aliphatic hydrocarbon group or may be an aromatic hydrocarbon group.

### Aliphatic Hydrocarbon Group

The aliphatic hydrocarbon group indicates a hydrocarbon group that has no aromaticity. The aliphatic hydrocarbon group may be saturated or may be unsaturated (for example, an alkenylene group, an alkynylene group, or the like).

Examples of the aliphatic hydrocarbon group include a chain-like aliphatic hydrocarbon group, and an aliphatic hydrocarbon group containing a ring in the structure thereof. The chain-like aliphatic hydrocarbon group may be a linear aliphatic hydrocarbon group or may be a branched aliphatic hydrocarbon group.

### Linear or Branched Aliphatic Hydrocarbon Group

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable. Specific examples thereof include a methylene group [—CH₂—], an ethylene group [—(CH₂)₂—], a trimethylene group [—(CH₂)₃-], a tetramethylene group [—(CH₂)₄-], and a pentamethylene group [—(CH₂)₅-].

The branched aliphatic hydrocarbon group preferably has 2 to 10 carbon atoms, more preferably has 3 to 6 carbon atoms, still more preferably has 3 or 4 carbon atoms, and most preferably has 3 carbon atoms. As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

The linear or branched aliphatic hydrocarbon group may have or may not have a substituent.

### Aliphatic Hydrocarbon Group Having Ring in Structure Thereof

Examples of the aliphatic hydrocarbon group having a ring in the structure thereof include a cyclic aliphatic hydrocarbon group which may have a substituent having a heteroatom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. As the linear or branched aliphatic hydrocarbon group, the same groups as those described above are exemplary examples.

The cyclic aliphatic hydrocarbon group preferably has 3 to 20 carbon atoms and more preferably has 3 to 12 carbon atoms.

The cyclic aliphatic hydrocarbon group may be a polycyclic group or a monocyclic group. The monocyclic alicyclic hydrocarbon group is preferably a group obtained by removing two hydrogen atoms from a monocycloalkane. The monocycloalkane is preferably a monocycloalkane having 3 to 6 carbon atoms, and specific examples thereof include cyclopropane, cyclopentane, and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples of the polycycloalkane include adamantane, norbornane, isobornane, tricyclodecane, and tetracyclododecane.

The cyclic aliphatic hydrocarbon group may or may not have a substituent. Examples of the substituent include an alkyl group, an alkoxy group, a halogen atom, a halogenated alkyl group, a hydroxy group, and a carbonyl group.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂—O— is preferable.

### Aromatic Hydrocarbon Group

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the number of carbon atoms.

Specifically, as the aromatic ring, an aromatic hydrocarbon ring such as benzene, naphthalene, anthracene, and phenanthrene; and an aromatic heterocyclic ring in which some carbon atoms constituting the aromatic hydrocarbon ring have been substituted with heteroatoms are exemplary examples. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group in which two hydrogen atoms have been removed from the above-described aromatic hydrocarbon ring or aromatic heterocyclic ring (an arylene group or a heteroarylene group); a group in which two hydrogen atoms have been removed from an aromatic compound having two or more aromatic rings (such as biphenyl or fluorene); and a group in which one hydrogen atom of a group (an aryl group or a heteroaryl group) obtained by removing one hydrogen atom from the above-described aromatic hydrocarbon ring or aromatic heterocyclic ring has been substituted with an alkylene group (for example, a group obtained by further removing one hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aryl group or the heteroaryl group is preferably in a range of 1 to 4, more preferably 1 or 2, and particularly preferably 1.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of the substituent include an alkyl group, an alkoxy group, a halogen atom, a halogenated alkyl group, and a hydroxy group.

### Divalent Linking Group Having Heteroatom:

Examples of the divalent linking group having a heteroatom as X⁰¹ include —O—, —C(═O)—O—, —O—C(═O)—, —C(═O)—, —O—C(═O)—O—, —C(═O)—NH—, —C(═O)—NH—C(═O)—, —NH—, —NH—C(═NH)—(H may be substituted with a substituent such as a hydrocarbon group, an acyl group, or an alkoxyalkyl group), —S—, —S(═O)₂—, —S(═O)₂—O—, and a group represented by General Formula —Y²¹—O—Y²²—, —Y²¹—O—, —Y²¹—C(═O)—O—, —C(═O)—O—Y²¹—, —[Y²¹—C(═O)—O]ₘ-Y22-, —Y²¹—O—C(═O)—Y²²—, or —Y²¹—S(═O)₂—O—Y²²—[in the formulae, Y²¹ and Y²² each independently represent a divalent hydrocarbon group which may have a substituent, 0 represents an oxygen atom, and m″ represents an integer of 0 to 3].

In a case where the divalent linking group having a heteroatom is —C(═O)—NH—, —C(═O)—NH—C(═O)—, —NH—, or —NH—C(═NH)—, H of the divalent linking group may be substituted with a substituent such as a hydrocarbon group, an acyl group, or an alkoxyalkyl group. The number of carbon atoms in the substituent is preferably in a range of 1 to 10, more preferably in a range of 1 to 8, and particularly preferably in a range of 1 to 5.

In General Formula —Y²¹-Y²²—, —Y²¹—, —Y²¹—C(═O)—O—, —C(═O)—O—Y²¹—, —[Y²¹—C(═O)—O]ₘ, —Y²²—, —Y²¹—O—C(═O)—Y²²—, or —Y²¹—S(═O)₂—O—Y²²—, Y²¹ and Y²² each independently represent a divalent hydrocarbon group which may have a substituent. As the divalent hydrocarbon group, the same ones as those described above are exemplary examples.

Y²¹ and Y²² each represent preferably a linear aliphatic hydrocarbon group, more preferably a linear alkylene group, still more preferably a linear alkylene group having 1 to 5 carbon atoms, and particularly preferably a methylene group or an ethylene group.

Alternatively, Y²¹ and Y²² each represent preferably a linear or branched aliphatic hydrocarbon group or a linear or branched fluorinated aliphatic hydrocarbon group and more preferably a linear or branched alkylene group having 1 to 5 carbon atoms or a linear or branched fluorinated alkylene group.

X⁰¹ represents preferably a cyclic aliphatic hydrocarbon group which may have a substituent, an aromatic hydrocarbon group which may have a substituent, an ester bond [—C(═O)—O— or —O—C(═O)—], an ether bond (—O—), an amide bond [—NH—C(═O)— or —C(═O)—NH—], a linear or branched aliphatic hydrocarbon group, a linear or branched fluorinated aliphatic hydrocarbon group, a combination of two or more thereof, or a single bond and more preferably a cyclic aliphatic hydrocarbon group which may have a substituent, an aromatic hydrocarbon group which may have a substituent, an ester bond [—C(═O)—O— or —O—C(═O)—], a linear or branched aliphatic hydrocarbon group, a linear or branched fluorinated aliphatic hydrocarbon group, or a combination of two or more thereof.

In Formula (a01-b), R⁰¹ and R⁰² each independently represent preferably a fluorinated alkyl group having 1 to 5 carbon atoms, a fluorine atom, or a hydrogen atom, more preferably a fluorinated alkyl group having 1 to 5 carbon atoms or a fluorine atom, and still more preferably a fluorine atom.

Suitable examples of the constitutional unit (a01) include a constitutional unit represented by General Formula (a01-b0).

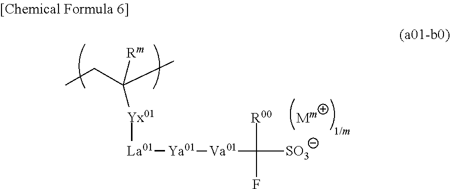

[In the formula, R^{m} represents an alkyl group having 1 to 5 carbon atoms, a halogenated alkyl group having 1 to 5 carbon atoms, a halogen atom, or a hydrogen atom. Yx⁰¹ represents a divalent linking group or a single bond. La⁰¹ represents a hydrocarbon group which may have a substituent. Ya⁰¹ represents a divalent linking group or a single bond. Va⁰¹ represents a fluorinated alkylene group, an alkylene group, or a single bond. Here, both Ya⁰¹ and Va⁰¹ do not represent a single bond at the same time. R⁰⁰ represents a fluorinated alkyl group having 1 to 5 carbon atoms, a fluorine atom, or a hydrogen atom. M^{m+} represents an m-valent onium cation. m represents an integer of 1 or greater.]

In Formula (a01-b0), as the alkyl group having 1 to 5 carbon atoms as R^{m}, a linear or branched alkyl group having 1 to 5 carbon atoms is preferable. Specific examples thereof include a methyl group, an ethyl group, a propyl group, an isopropyl group, an n-butyl group, an isobutyl group, a tert-butyl group, a pentyl group, an isopentyl group, and a neopentyl group.

The halogenated alkyl group having 1 to 5 carbon atoms as R^{m} is a group in which some or all hydrogen atoms in the alkyl group having 1 to 5 carbon atoms are substituted with halogen atoms. The halogen atom is preferably a fluorine atom.

Examples of the halogen atom as R^{m} include a fluorine atom, a chlorine atom, a bromine atom, and an iodine atom.

It is preferable that R^{m} represents a hydrogen atom or a methyl group from the viewpoint of the industrial availability.

In Formula (a01-b0), examples of the divalent linking group as Yx⁰¹ include the same groups as those for the divalent linking group described in the section of the divalent linking group as Ya^{x0} above.

Specific examples of Yx⁰¹ include an ester bond (—C(═O)—O—), an oxycarbonyl group (—O—C(═O)—), an ether bond (—O—), a linear or branched alkylene group, an arylene group which may have a substituent, an optional combination of these groups, and a single bond. Among these, a combination of an arylene group which may have a substituent and an ester bond (—C(═O)—O—), an ester bond (—C(═O)—O—), or a single bond is preferable, and a combination of a phenylene group which may have a substituent and an ester bond (—C(═O)—O—), or an ester bond (—C(═O)—O—) is more preferable.

In Formula (a01-b0), examples of La⁰¹ include the same groups as those for the aliphatic hydrocarbon group (the chain-like aliphatic hydrocarbon group and the aliphatic hydrocarbon group having a ring in the structure) and the aromatic hydrocarbon group described in the section of the divalent hydrocarbon group which may have a substituent as X⁰¹ above. Among these, an aliphatic hydrocarbon group having a ring in the structure or an aromatic hydrocarbon group is preferable, and a cyclic aliphatic hydrocarbon group (group obtained by removing two hydrogen atoms from an aliphatic hydrocarbon ring) which may have a substituent or an aromatic hydrocarbon group which may have a substituent is more preferable.

The cyclic aliphatic hydrocarbon group as La⁰¹ may be a monocyclic alicyclic hydrocarbon group or a polycyclic alicyclic hydrocarbon group, preferably a polycyclic alicyclic hydrocarbon group, more preferably a group obtained by removing two hydrogen atoms from a polycycloalkane, and still more preferably a group obtained by removing two hydrogen atoms from adamantane.

The cyclic aliphatic hydrocarbon group may or may not have a substituent. Examples of the substituent include an alkyl group, an alkoxy group, a halogen atom, a halogenated alkyl group, a hydroxy group, and a carbonyl group.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂—O— is preferable.

As the aromatic hydrocarbon group as La⁰¹, a group (an arylene group or a heteroarylene group) obtained by removing two hydrogen atoms from an aromatic hydrocarbon ring or an aromatic heterocyclic ring is preferable, an arylene group is more preferable, and a phenylene group is still more preferable.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of the substituent include an alkyl group, an alkoxy group, a halogen atom, a halogenated alkyl group, and a hydroxy group.

Among these, La⁰¹ represents preferably a cyclic aliphatic hydrocarbon group which may have a substituent and more preferably a polycyclic alicyclic hydrocarbon group which may have a substituent.

In Formula (a01-b0), examples of the divalent linking group as Ya⁰¹ include the same groups as those for the divalent linking group having a heteroatom as X⁰¹ above.

Ya⁰¹ represents preferably an oxygen atom (ether bond: —O—), an ester bond (—C(═O)—O—), or an oxycarbonyl group (—O—C(═O)—) and more preferably (La⁰¹ side)-C(═O)—O—(Va⁰¹ side).

In Formula (a01-b0), examples of the alkylene group as Va⁰¹ include the same groups as those for the linear or branched aliphatic hydrocarbon group as X⁰¹ described above. Examples of the fluorinated alkylene group as Va⁰¹ include a group in which some or all of the hydrogen atoms constituting the alkylene group as Va⁰¹ are substituted with fluorine atoms.

It is preferable that Va⁰¹ represents a methylene group (—CH₂—) or —CH(CF₃)—.

In Formula (a01-b0), R⁰⁰ represents a fluorinated alkyl group having 1 to 5 carbon atoms, a fluorine atom, or a hydrogen atom, preferably a fluorinated alkyl group having 1 to 5 carbon atoms or a fluorine atom, and more preferably a fluorine atom.

Specific examples of the constitutional unit (a01) are shown below, but the examples are not limited thereto.

In each formula, R^{α} represents a trifluoromethyl group, a methyl group, or a hydrogen atom. M^{m+} represents an m-valent onium cation. m represents an integer of 1 or greater.

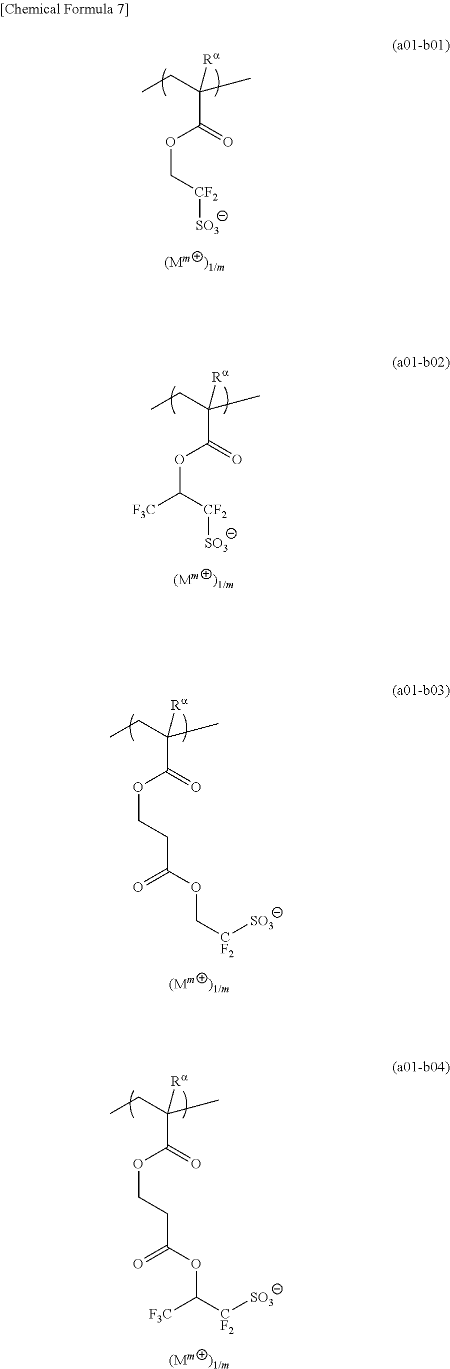

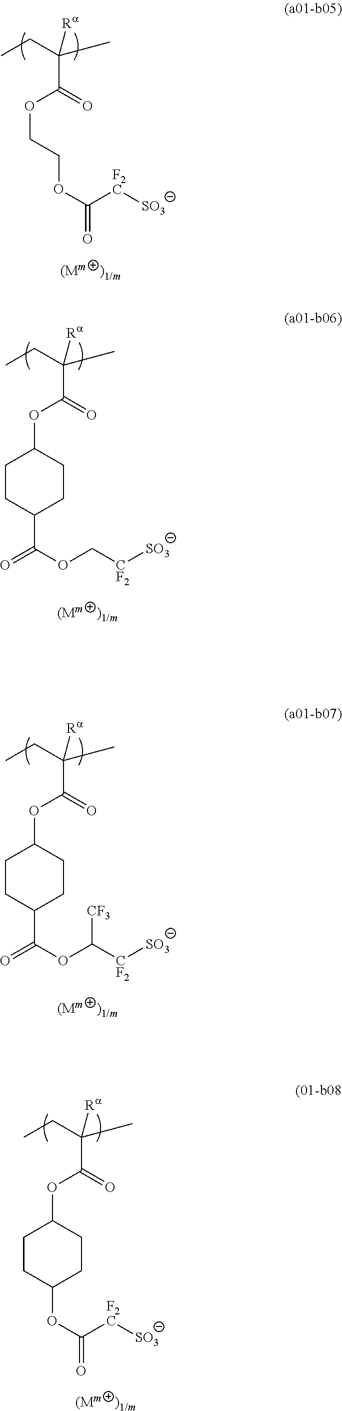

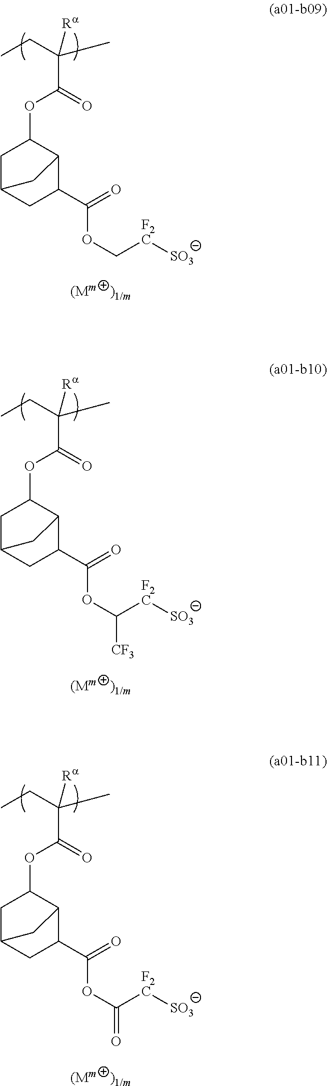

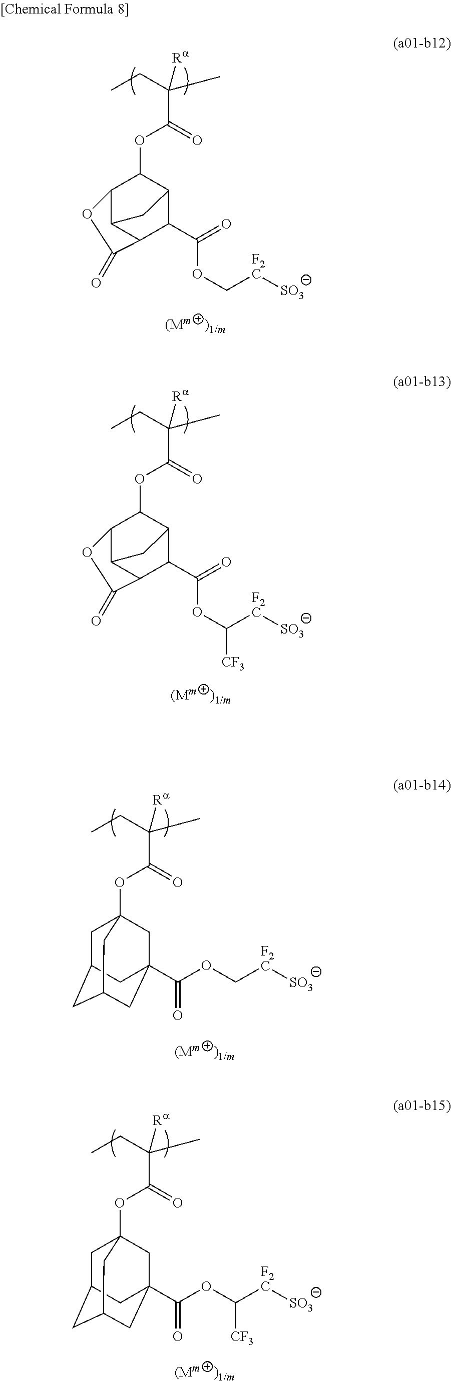

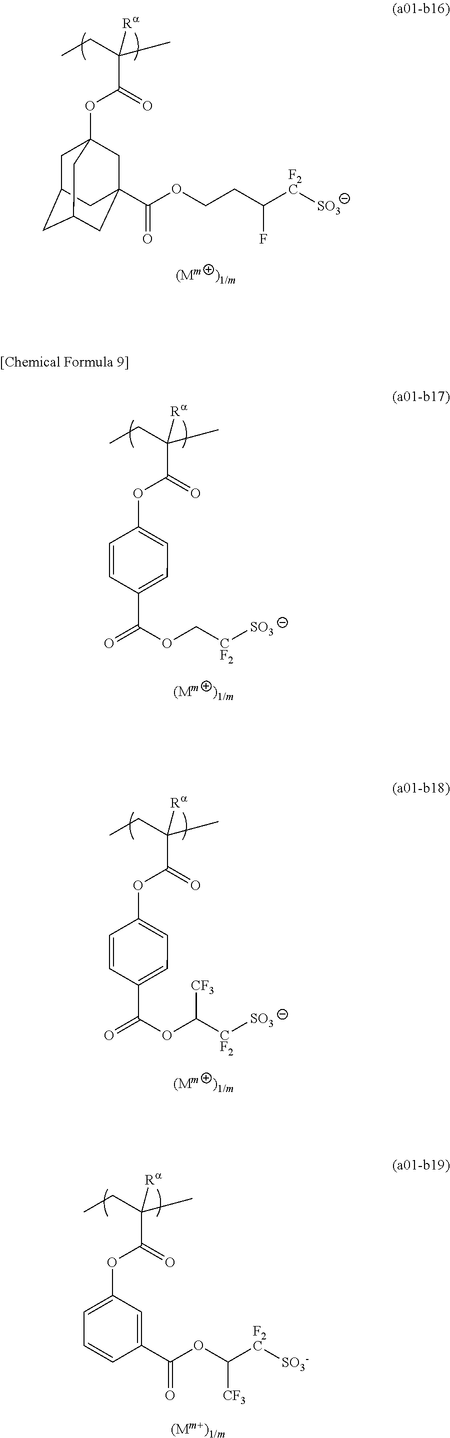

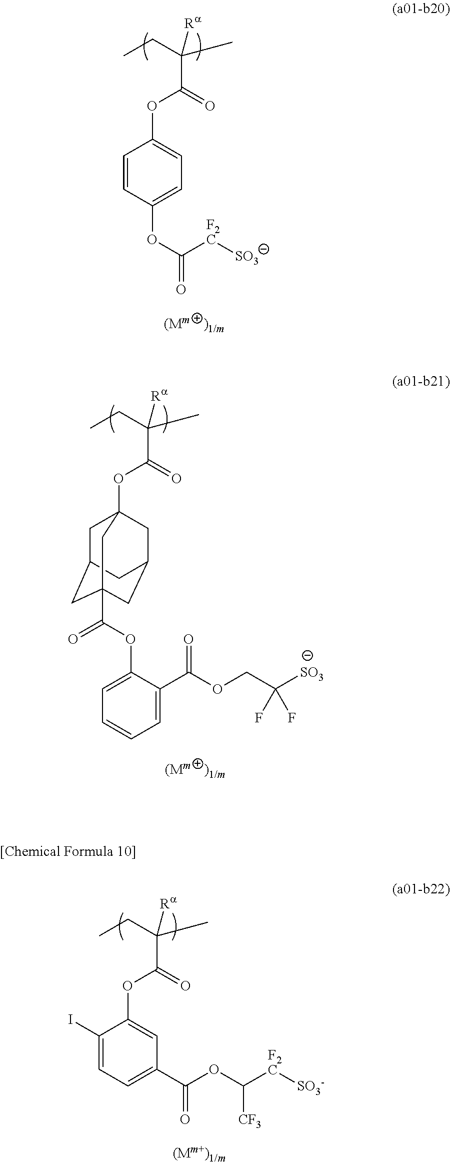

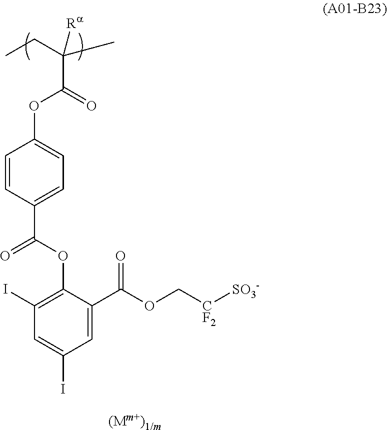

The constitutional unit (a01) is preferably a constitutional unit selected from the group consisting of a constitutional unit represented by any of Formulae (a01-b01) to (a01-b05), a constitutional unit represented by any of Formulae (a01-b06) to (a01-b16), and a constitutional unit represented by any of Formulae (a01-b17) to (a01-b23), more preferably a constitutional unit selected from the group consisting of a constitutional unit represented by any of Formulae (a01-b06) to (a01-b16) and a constitutional unit represented by any of Formulae (a01-b17) to (a01-b23), still more preferably a constitutional unit selected from the group consisting of a constitutional unit represented by any of Formulae (a01-b06) to (a01-b16), and particularly preferably a constitutional unit selected from the group consisting of a constitutional unit represented by any of Formulae (a01-b09) to (a01-b16).

### {Cation Moiety}

In Formulae (a01-b) and (a01-b0), M^{m+} represents an m-valent onium cation. m represents an integer of 1 or greater. Among the onium cations as M^{m+}, a sulfonium cation or an iodonium cation is preferable, and a sulfonium cation is more preferable.

Preferred examples of the cation moiety (M^{m+})_{1/m} include onium cations each represented by General Formulae (ca-1) to (ca-3).

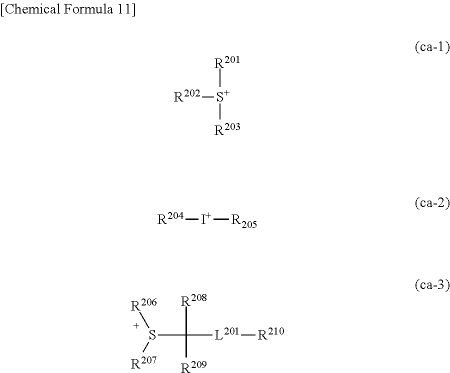

[In the formula, R²⁰¹ to R²⁰⁷ each independently represent an aryl group which may have a substituent, an alkyl group which may have a substituent, or an alkenyl group which may have a substituent. R²⁰¹ to R²⁰³, and R²⁰⁶ and R²⁰⁷ may be bonded to each other to form a ring together with the sulfur atoms in the formulae. R²⁰⁸ and R²⁰⁹ each independently represent a hydrogen atom or an alkyl group having 1 to 5 carbon atoms. R²¹⁰ represents an aryl group which may have a substituent, an alkyl group which may have a substituent, an alkenyl group which may have a substituent, or a —SO₂-containing cyclic group which may have a substituent. L²⁰¹ represents —C(═O)— or —C(═O)—O—.]

In General Formulae (ca-1) to (ca-3), examples of the aryl group as R²⁰¹ to R²⁰⁷ include an unsubstituted aryl group having 6 to 20 carbon atoms, where a phenyl group or a naphthyl group is preferable.

The alkyl group as R²⁰¹ to R²⁰⁷ is preferably a chain-like or cyclic alkyl group which has 1 to 30 carbon atoms.

The alkenyl group as R²⁰¹ to R²⁰⁷ preferably has 2 to 10 carbon atoms.

Examples of the substituent that R²⁰¹ to R²⁰⁷ and R²¹⁰ may have include an alkyl group, a halogen atom, a halogenated alkyl group, a carbonyl group, a cyano group, an amino group, an aryl group, and a group represented by any of General Formulae (ca-r-1) to (ca-r-8). Among these, an alkyl group, a halogen atom, a halogenated alkyl group, or a group represented by any of General Formulae (ca-r-1) to (ca-r-8) is preferable, and a chain-like alkyl group, an iodine atom, a fluorine atom, a fluorinated alkyl group, or a group represented by General Formula (ca-r-3) is more preferable.

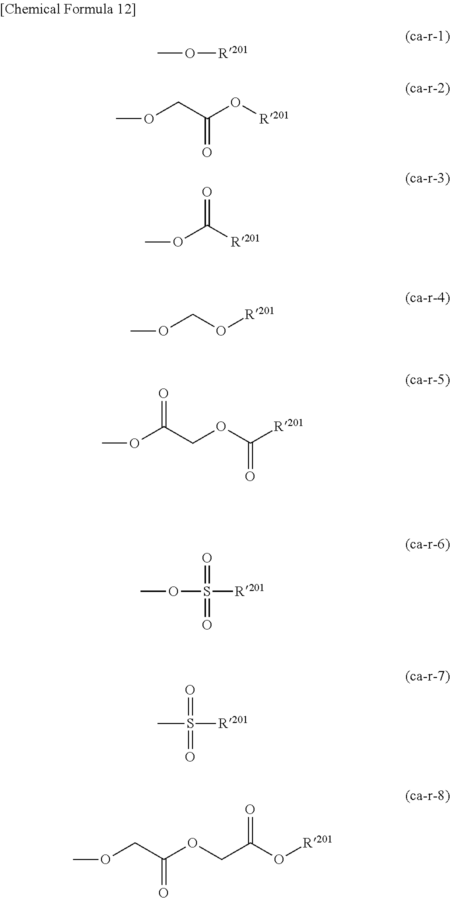

[In the formulae, R′²⁰¹'s each independently represents a hydrogen atom, a cyclic group which may have a substituent, a chain-like alkyl group which may have a substituent, or a chain-like alkenyl group which may have a substituent.]

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group is saturated.

The aromatic hydrocarbon group as R′²⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as R′²⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R′²⁰¹ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclodecane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R′²⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear or branched aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable. Specific examples thereof include a methylene group [—CH₂—], an ethylene group [—(CH₂)₂-], a trimethylene group [—(CH₂)₃-], a tetramethylene group [—(CH₂)₄-], and a pentamethylene group [—(CH₂)₅-].

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Further, the cyclic hydrocarbon group as R′²⁰¹ may have a heteroatom such as a heterocyclic ring. Specific examples thereof include lactone-containing cyclic groups each represented by General Formulae (a2-r-1) to (a2-r-7), —SO₂-containing cyclic groups each represented by General Formulae (b5-r-1) to (b5-r-4), and other heterocyclic groups each represented by Chemical Formulae (r-hr-1) to (r-hr-16).

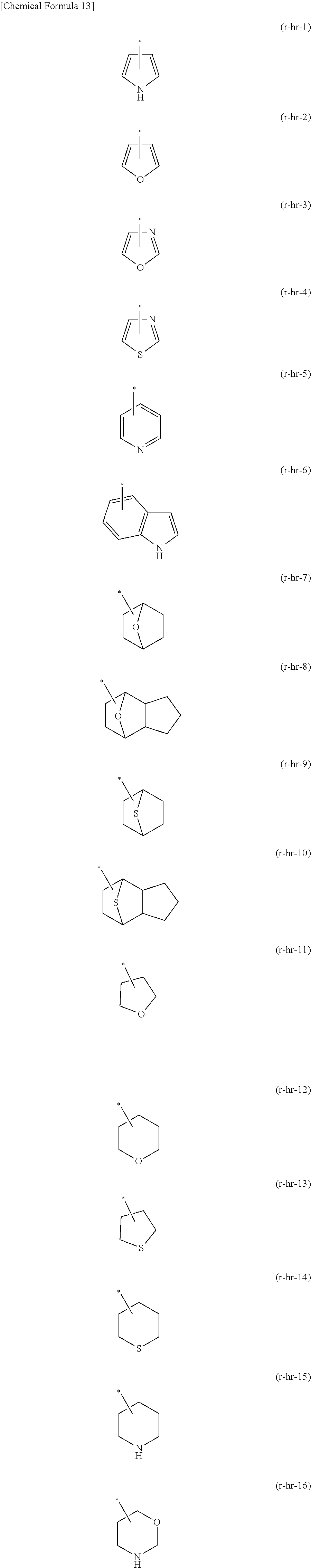

Examples of the substituent for the cyclic group as R′²⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

As the halogen atom as a substituent, a fluorine atom or an iodine atom is preferable.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group are substituted with the above-described halogen atoms.

The carbonyl group as the substituent is a group that substitutes a methylene group (—CH₂—) constituting the cyclic hydrocarbon group.

Chain-Like Alkyl Group which May have Substituent:

The chain-like alkyl group as R′²⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-like alkenyl group which may have substituent:

The chain-like alkenyl group as R′²⁰¹ may be linear or branched, and has preferably 2 to 10 carbon atoms, more preferably 2 to 5 carbon atoms, still more preferably 2 to 4 carbon atoms, and particularly preferably 3 carbon atoms. Examples of the linear alkenyl group include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹.

Examples of the cyclic group which may have a substituent, the chain-like alkyl group which may have a substituent, and the chain-like alkenyl group which may have a substituent as R′²⁰¹ include the same groups as those for the acid dissociable group represented by Formula (a1-r-2) described as the cyclic group which may have a substituent and the chain-like alkyl group which may have a substituent, in addition to those described above.

Among the examples, R′²⁰¹ represents preferably a cyclic group which may have a substituent and more preferably a cyclic hydrocarbon group which may have a substituent. More specific preferred examples thereof include a phenyl group, a naphthyl group, a group in which one or more hydrogen atoms have been removed from a polycycloalkane, a lactone-containing cyclic group represented by any of General Formulae (a2-r-1) to (a2-r-7), and a —SO₂-containing cyclic group represented by any of General Formulae (b5-r-1) to (b5-r-4).

As an example, from the viewpoint of high sensitivity in a case of forming a resist pattern, an aromatic hydrocarbon group which may have a substituent is preferable, an aromatic hydrocarbon group to which an iodine atom as a substituent is bonded is more preferable, and a benzene ring to which an iodine atom as a substituent is bonded is particularly preferable.

In General Formulae (ca-1) to (ca-3), in a case where R²⁰¹ to R²⁰³ and R²⁰⁶ and R²⁰⁷ are bonded to each other to form a ring with a sulfur atom in the formula, these groups may be bonded to each other via a heteroatom such as a sulfur atom, an oxygen atom, or a nitrogen atom, or a functional group such as a carbonyl group, —SO—, —SO₂—, —SO₃—, —COO—, —CONH—, or —N(R_{N})— (here, R_{N} represents an alkyl group having 1 to 5 carbon atoms). As a ring to be formed, a ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represent a hydrogen atom or an alkyl group having 1 to 5 carbon atoms and preferably a hydrogen atom or an alkyl group having 1 to 3 carbon atoms. In a case where R²⁰⁸ and R²⁰⁹ each represent an alkyl group, R²⁰⁸ and R²⁰⁹ may be bonded to each other to form a ring.

R²¹⁰ represents an aryl group which may have a substituent, an alkyl group which may have a substituent, an alkenyl group which may have a substituent, or a —SO₂— containing cyclic group which may have a substituent.

Examples of the aryl group as R²¹⁰ include an unsubstituted aryl group having 6 to 20 carbon atoms, and a phenyl group or a naphthyl group is preferable.

As the alkyl group as R²¹⁰, a chain-like or cyclic alkyl group having 1 to 30 carbon atoms is preferable.

The alkenyl group as R²¹⁰ preferably has 2 to 10 carbon atoms.

The —SO₂-containing cyclic group as R²¹⁰ is not particularly limited, and any —SO₂-containing cyclic group can be used. Specific examples thereof include groups each represented by General Formulae (b5-r-1) to (b5-r-4), where “—SO₂-containing polycyclic group” is preferable, and a group represented by General Formula (b5-r-1) is more preferable.

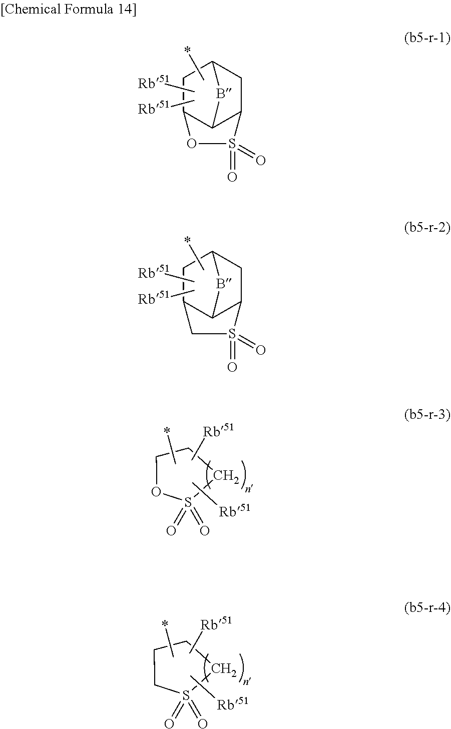

[In the formulae, each Rb′^{S1} independently represents a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, or an —SO₂-containing cyclic group; B″ represents an oxygen atom, a sulfur atom, or an alkylene group having 1 to 5 carbon atoms, which may contain an oxygen atom or a sulfur atom; and n′ represents an integer of 0 to 2. * represents a bonding site.]

In General Formulae (b5-r-1) and (b5-r-2), B″ represents an alkylene group having 1 to 5 carbon atoms which may have an oxygen atom or a sulfur atom, an oxygen atom, or a sulfur atom. B″ represents preferably an alkylene group having 1 to 5 carbon atoms or —O—, more preferably an alkylene group having 1 to 5 carbon atoms, and still more preferably a methylene group.

In General Formulae (b5-r-1) to (b5-r-4), Rb′⁵¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, and among the examples, it is preferable that Rb′⁵¹'s each independently represent a hydrogen atom or a cyano group.

Specific examples of the groups each represented by General Formulae (b5-r-1) to (b5-r-4) are shown below. In the formulae shown below, “Ac” represents an acetyl group.

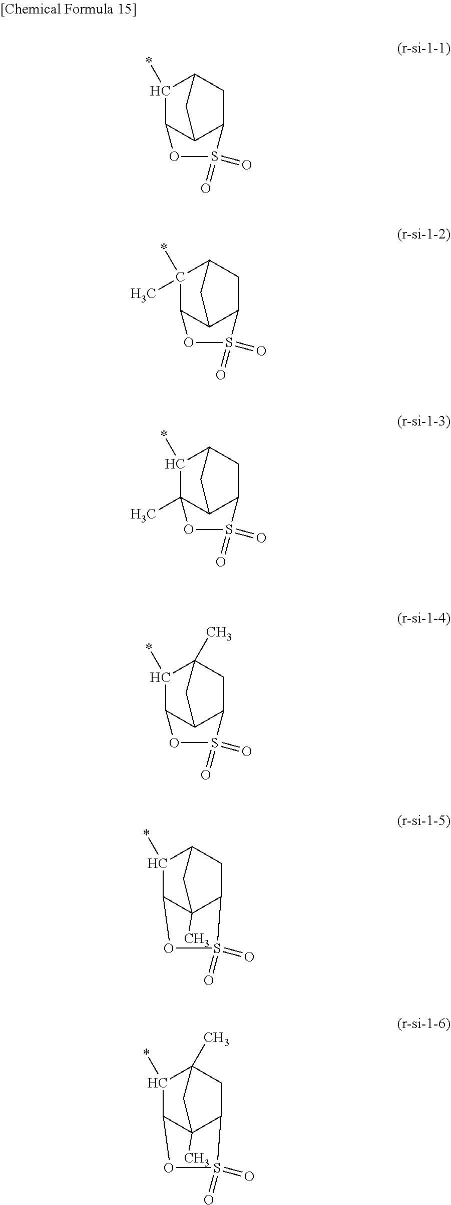

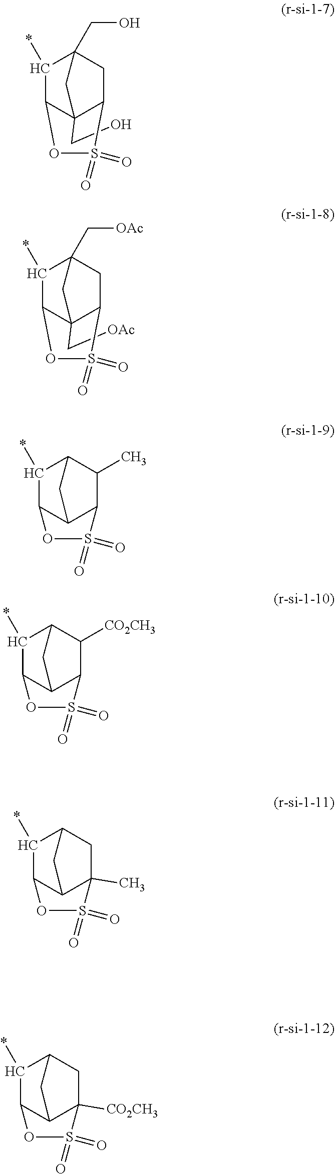

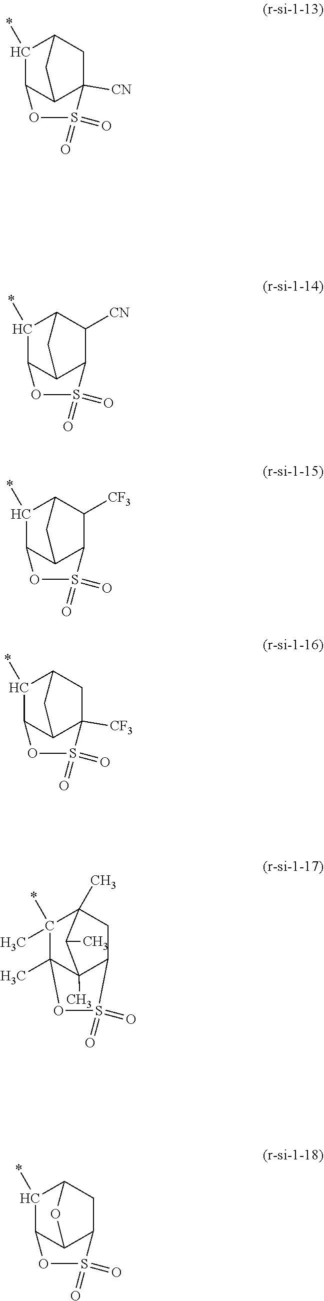

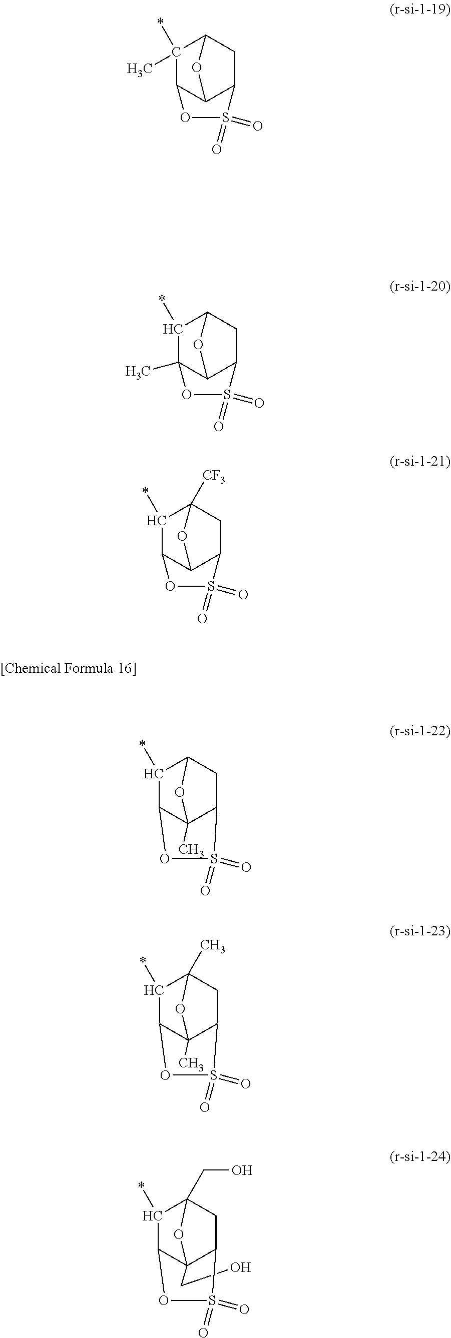

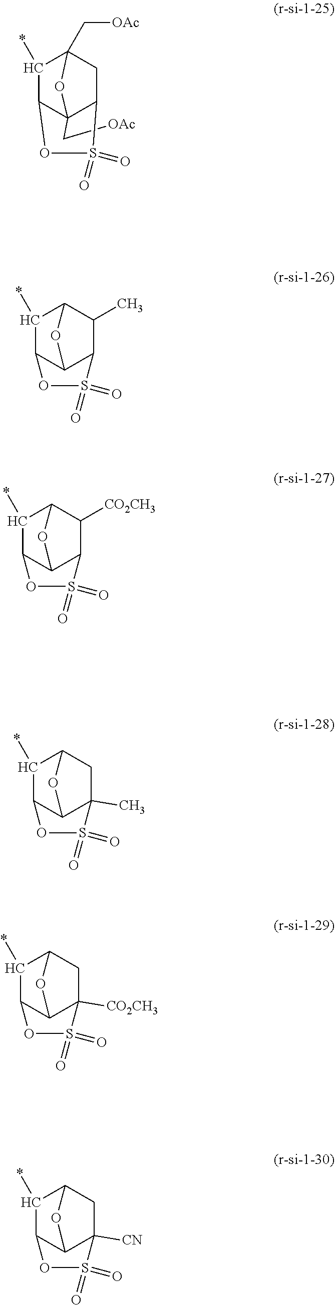

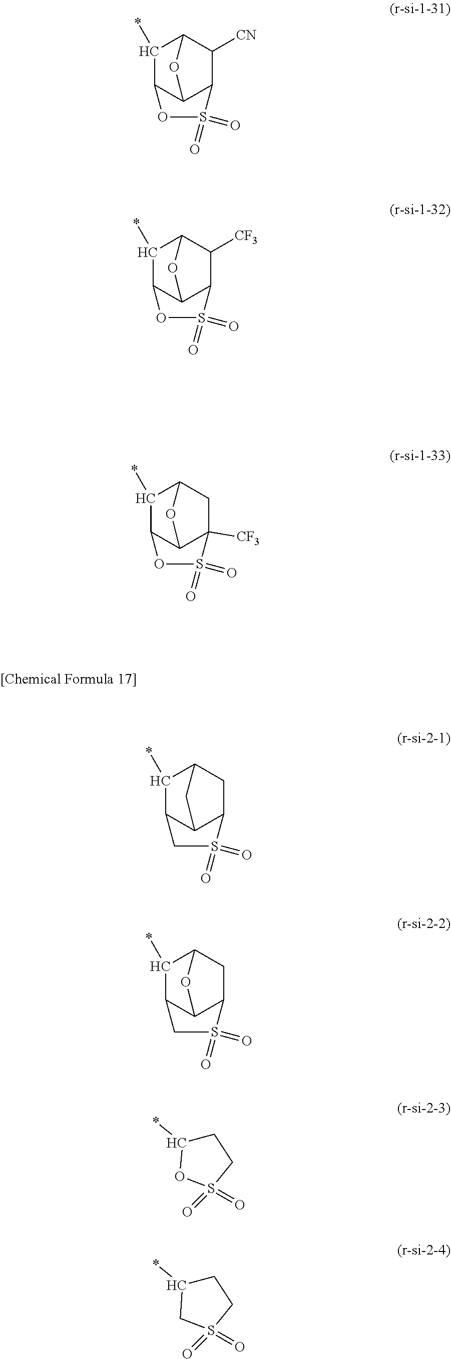

Specific examples of the suitable cation represented by General Formula (ca-1) include cations each represented by the following chemical formulae.

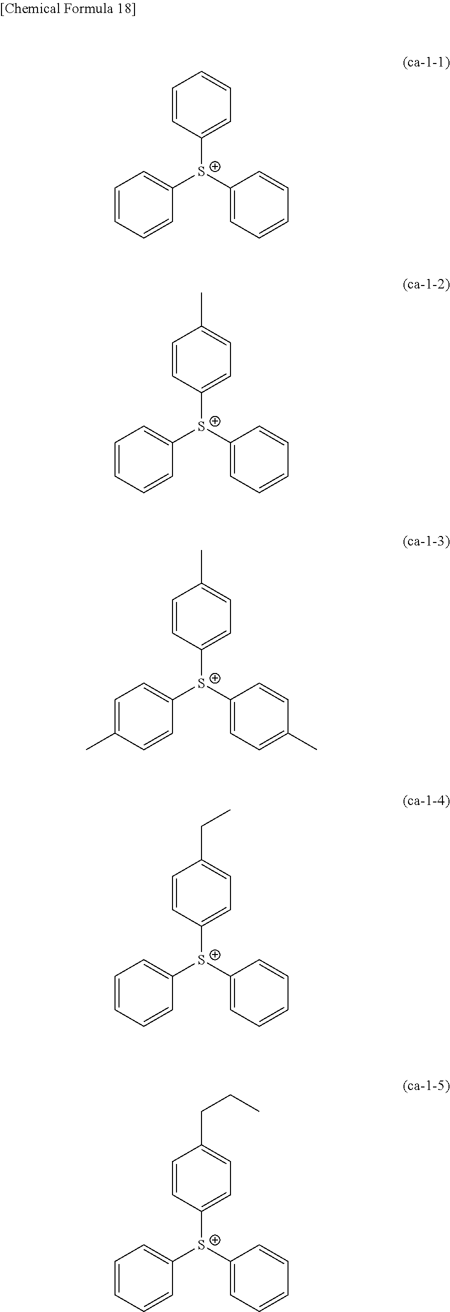

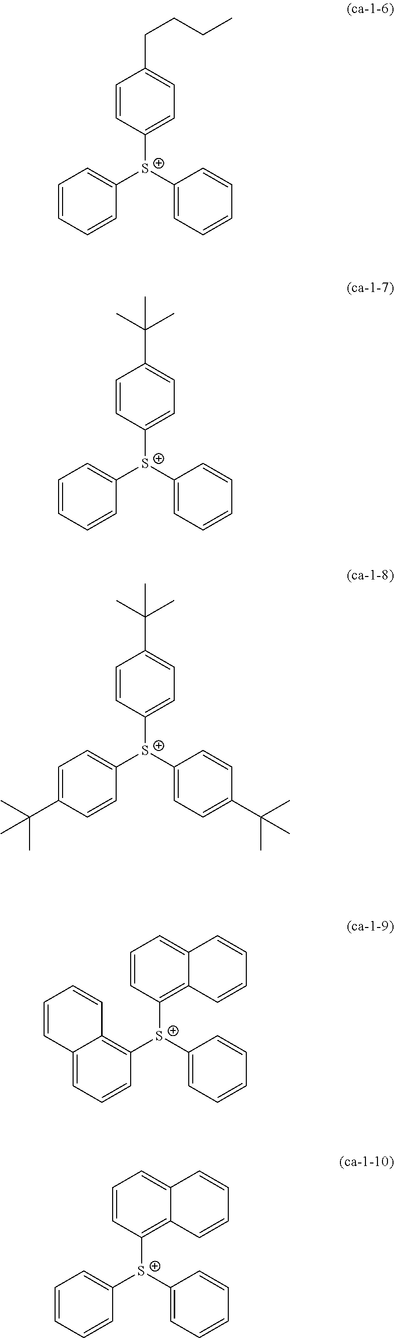

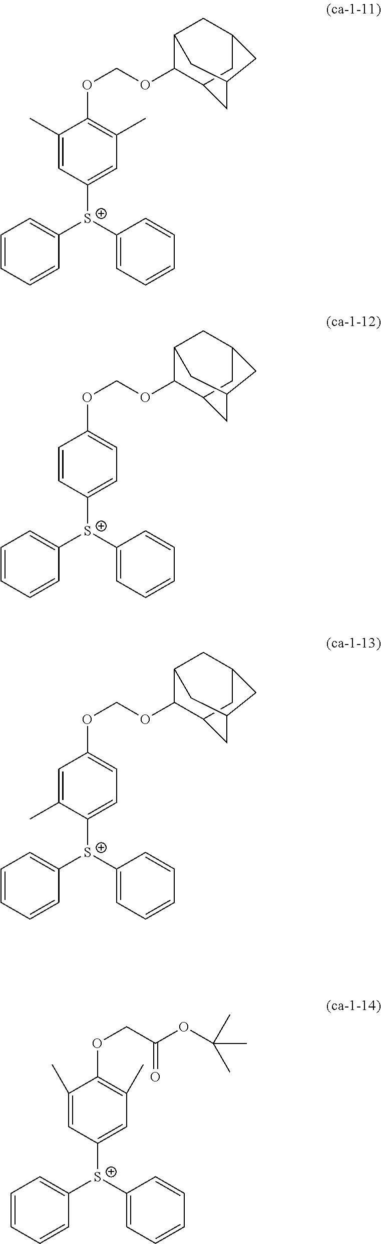

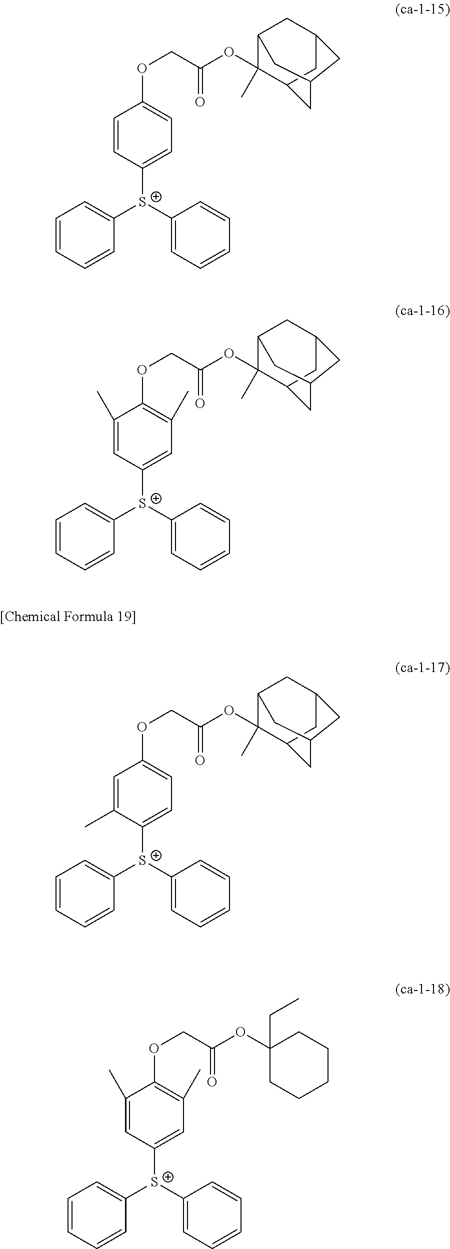

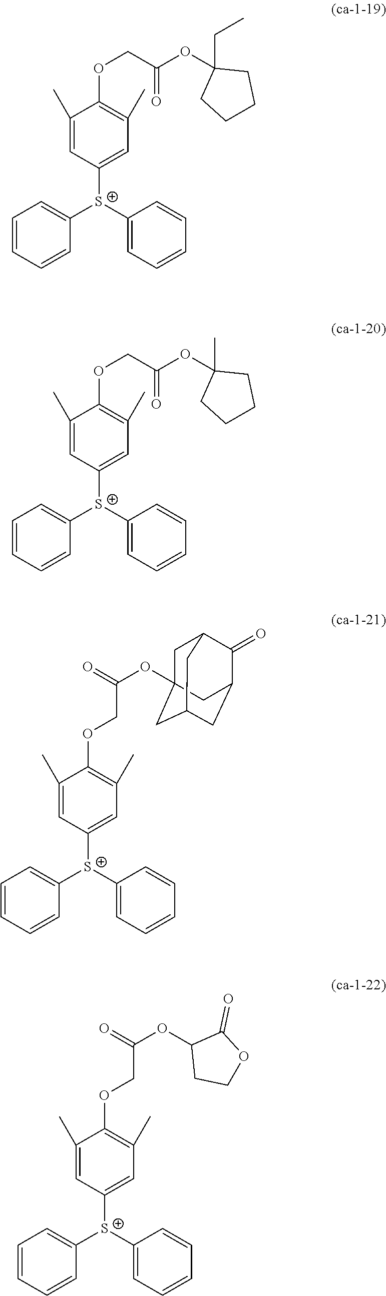

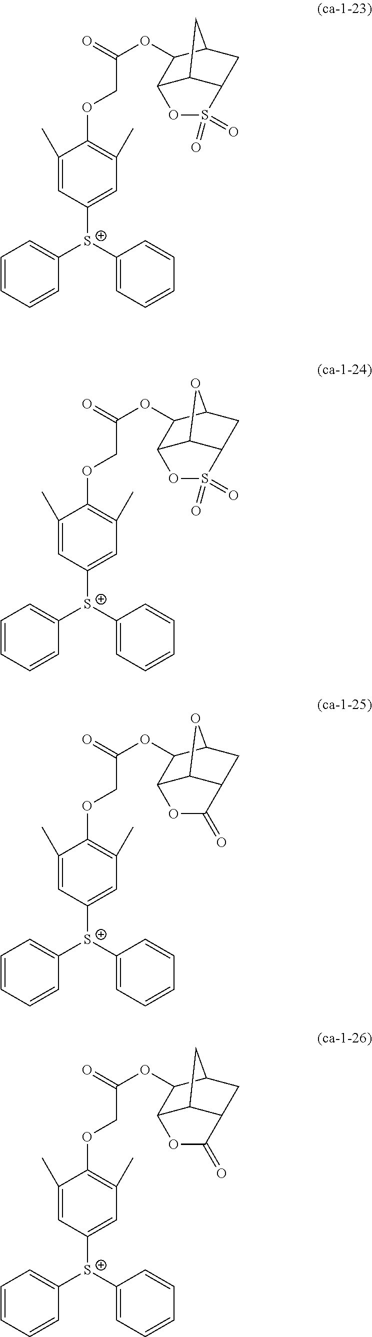

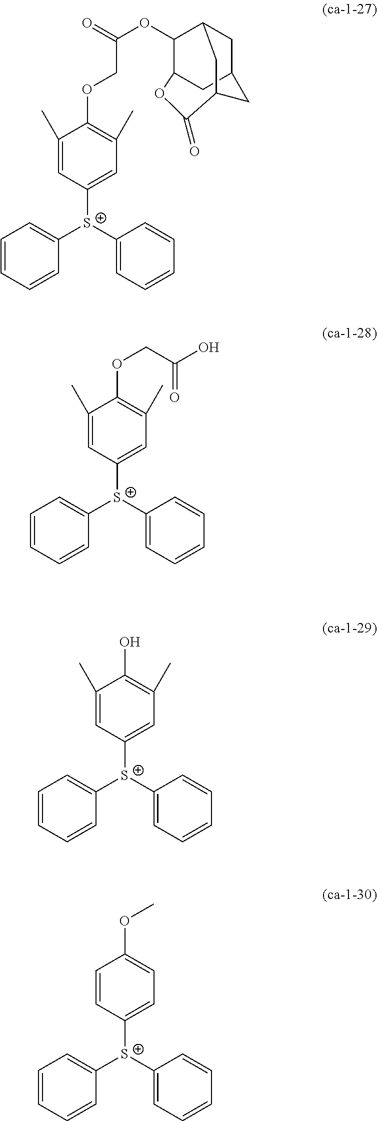

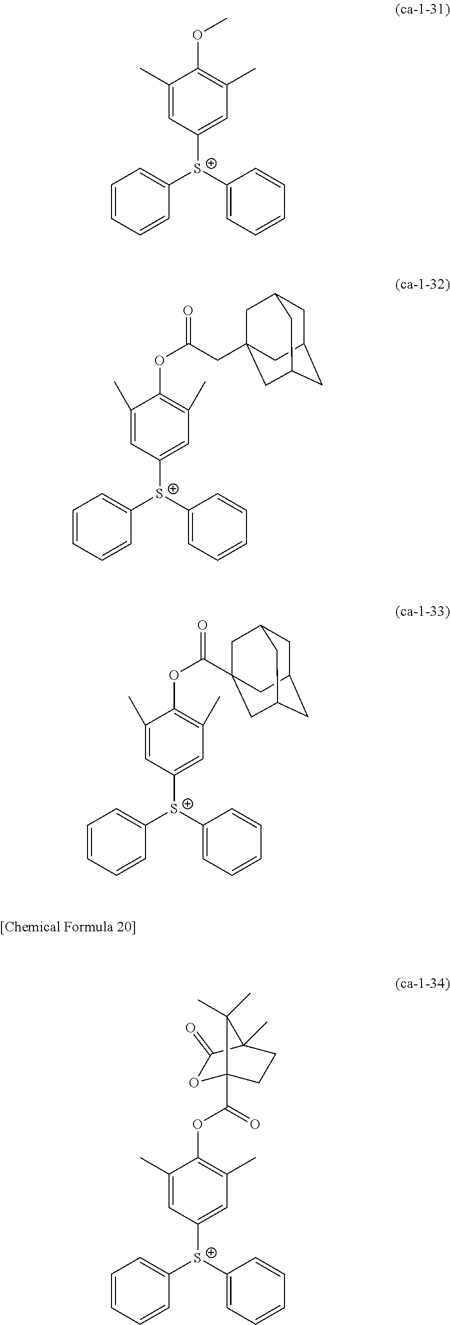

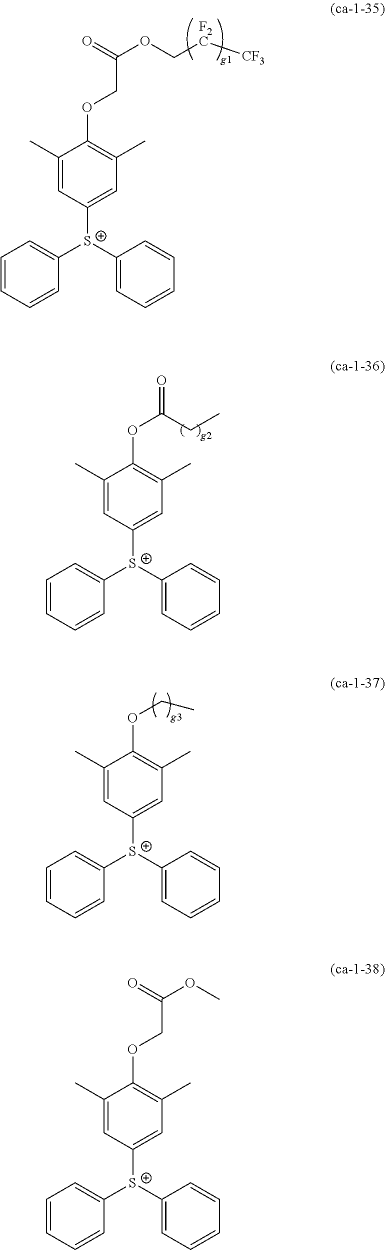

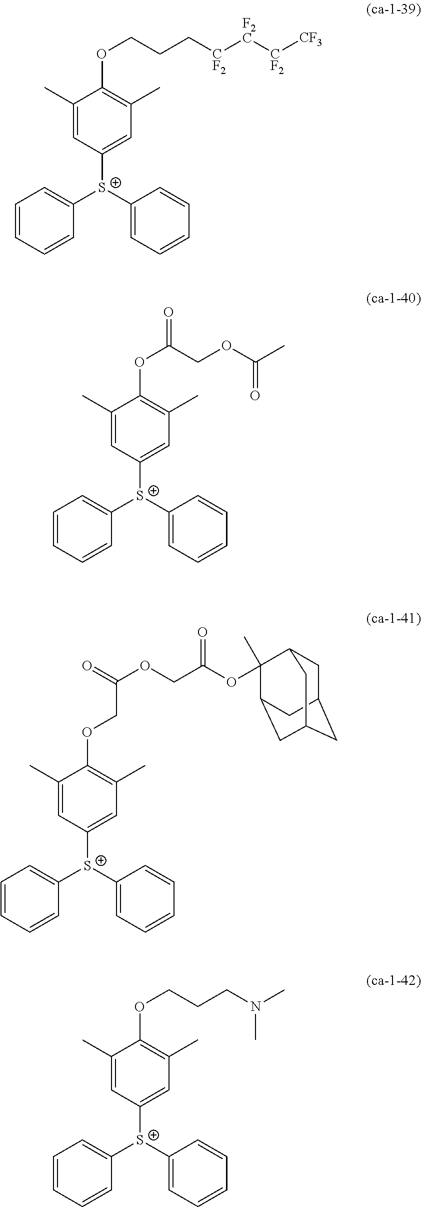

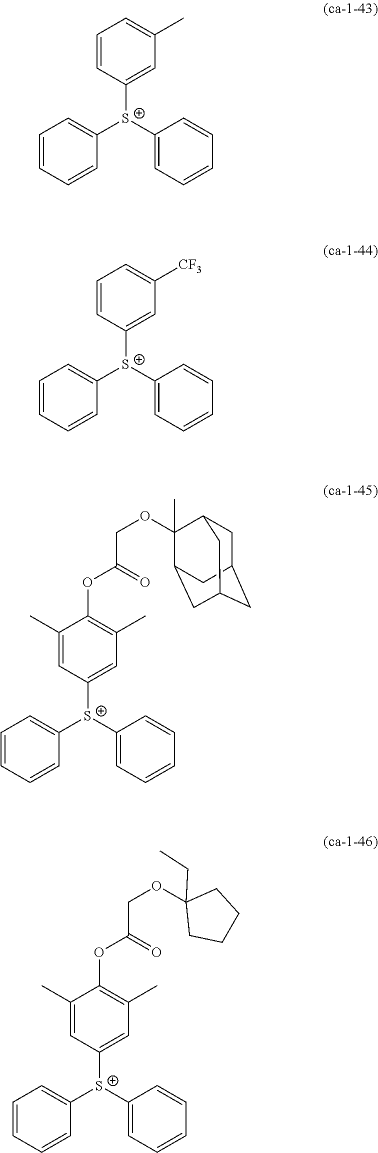

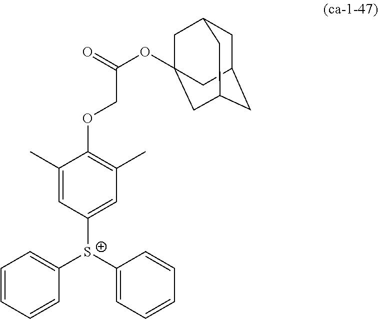

[In the formulae, g1, g2, and g3 represent a repeating number, g1 represents an integer of 1 to 5, g2 represents an integer of 0 to 20, and g3 represents an integer of 0 to 20.]

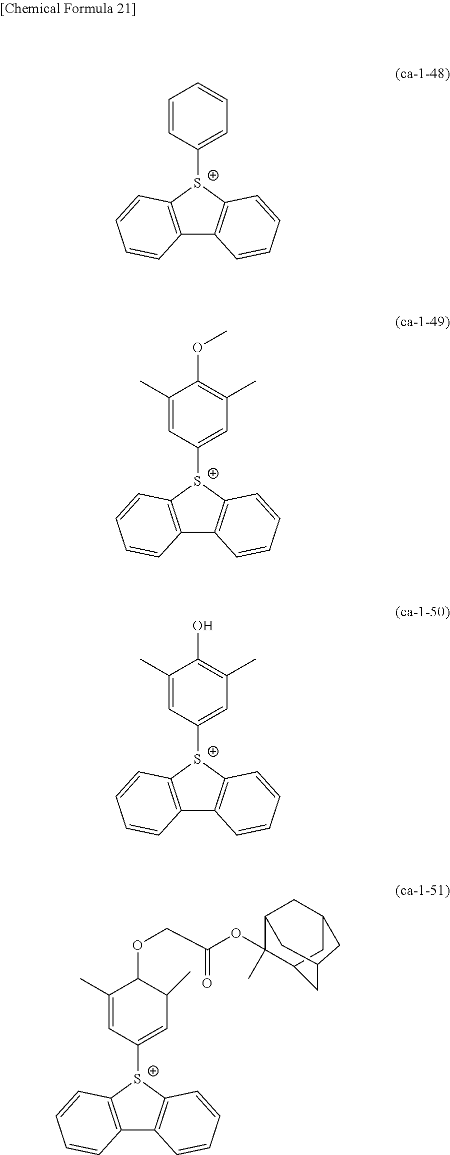 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; CC1=CC([s+]2c3ccccc3c3ccccc32)=CC(C)C1OCC(=O)OC1(C)C2CC3CC(C2)CC1C3]

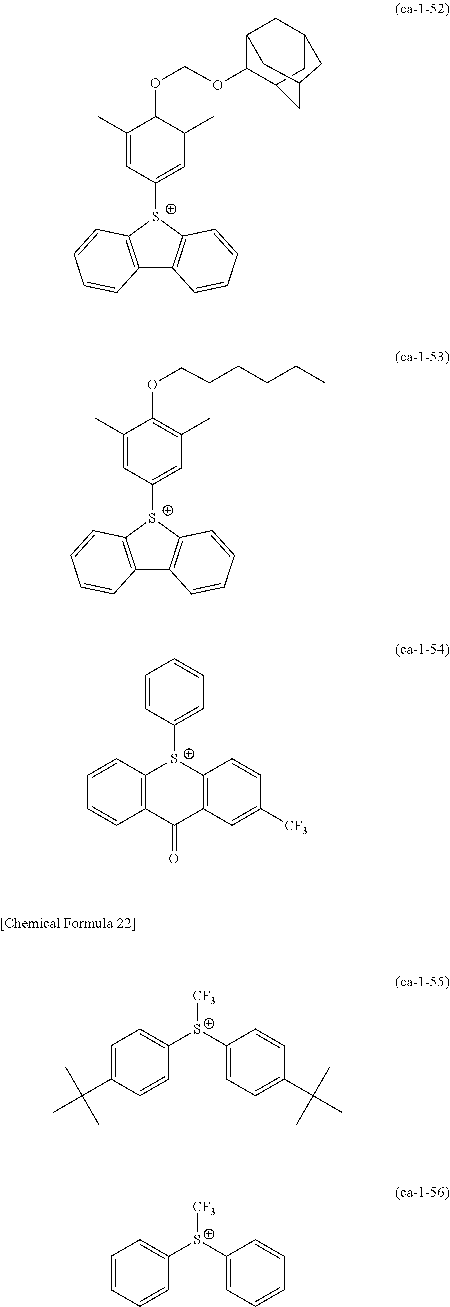

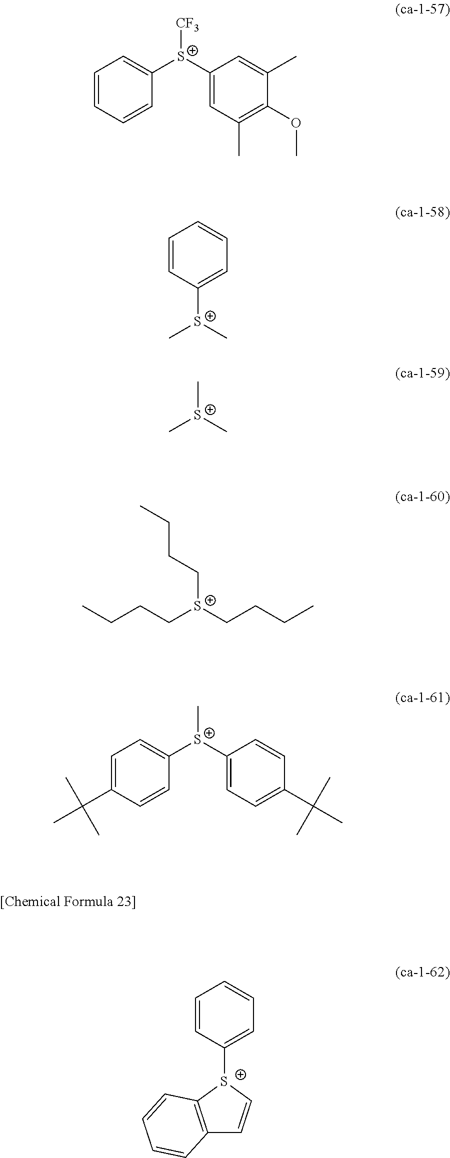

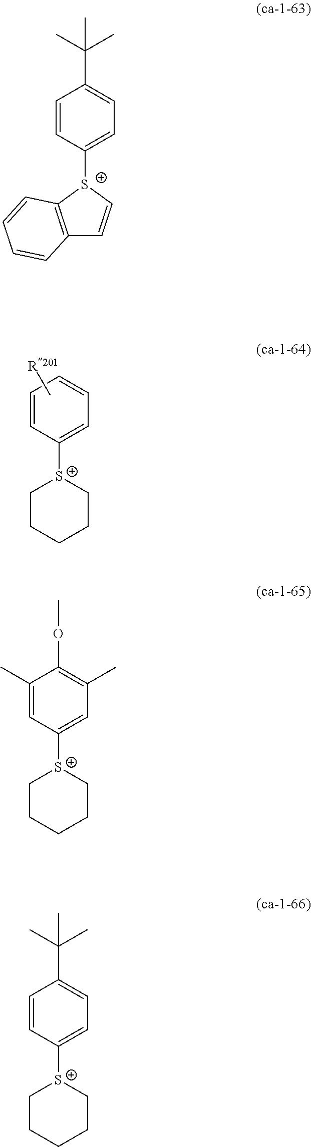

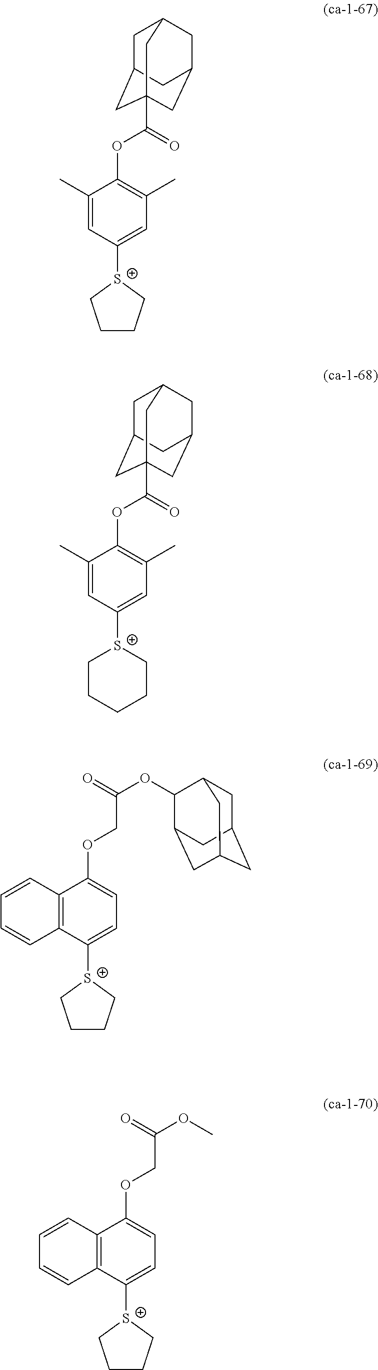 [SMILES: Cc1cc([S+]2CCCC2)cc(C)c1OC(=O)C12CC3CC(CC(C3)C1)C2 ; Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3 ; COC(=O)COc1ccc([S+]2CCCC2)c2ccccc12]

[In the formulae, R″²⁰¹ represents a hydrogen atom or a substituent, and examples of the substituent include the same groups as those for the substituents which may be included in R²⁰¹ to R²⁰⁷ and R²¹⁰ to R²¹².]

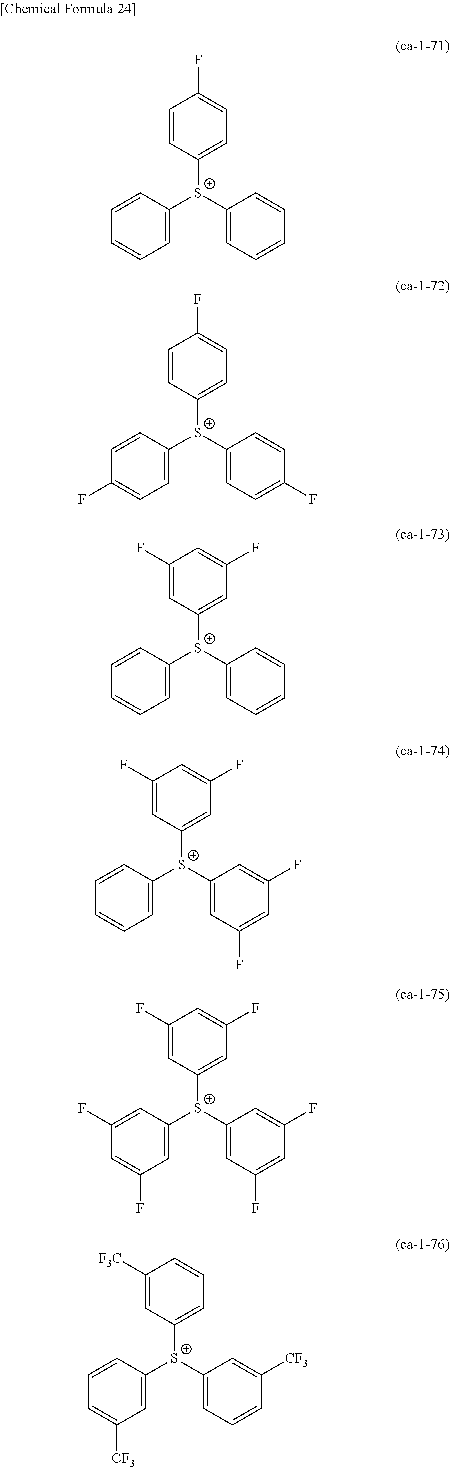 [SMILES: Fc1ccc([S+](c2ccccc2)c2ccccc2)cc1 ; Fc1ccc([S+](c2ccc(F)cc2)c2ccc(F)cc2)cc1 ; Fc1cc(F)cc([S+](c2ccccc2)c2ccccc2)c1 ; Fc1cc(F)cc([S+](c2ccccc2)c2cc(F)cc(F)c2)c1 ; Fc1cc(F)cc([S+](c2cc(F)cc(F)c2)c2cc(F)cc(F)c2)c1 ; FC(F)(F)c1cccc([S+](c2cccc(C(F)(F)F)c2)c2cccc(C(F)(F)F)c2)c1]

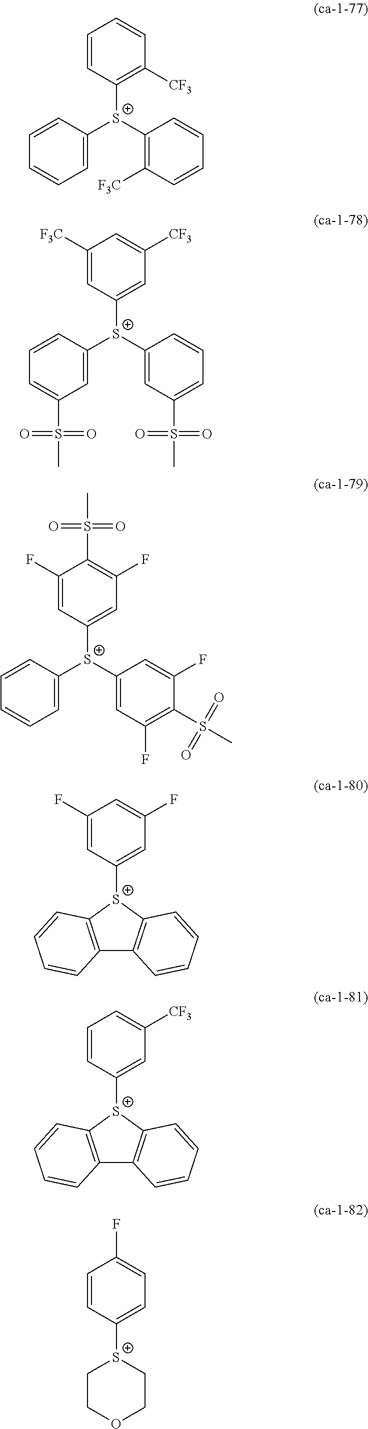 [SMILES: FC(F)(F)c1ccccc1[S+](c1ccccc1)c1ccccc1C(F)(F)F ; CS(=O)(=O)c1cccc([S+](c2cc(C(F)(F)F)cc(C(F)(F)F)c2)c2cccc(S(C)(=O)=O)c2)c1 ; CS(=O)(=O)c1c(F)cc([S+](c2ccccc2)c2cc(F)c(S(C)(=O)=O)c(F)c2)cc1F ; Fc1cc(F)cc(-[s+]2c3ccccc3c3ccccc32)c1 ; FC(F)(F)c1cccc(-[s+]2c3ccccc3c3ccccc32)c1 ; Fc1ccc([S+]2CCOCC2)cc1]

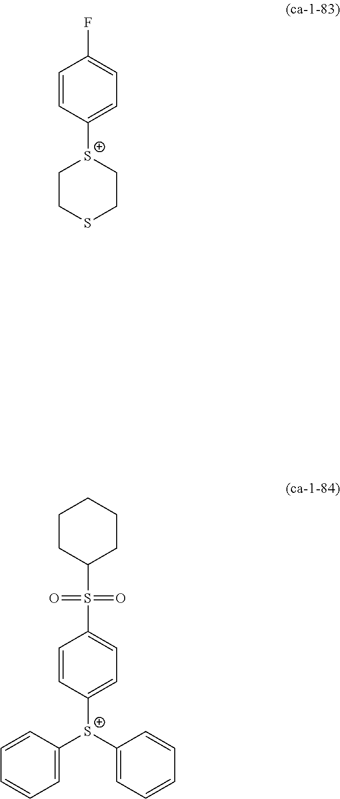 [SMILES: Fc1ccc([S+]2CCSCC2)cc1 ; O=S(=O)(c1ccc([S+](c2ccccc2)c2ccccc2)cc1)C1CCCCC1]

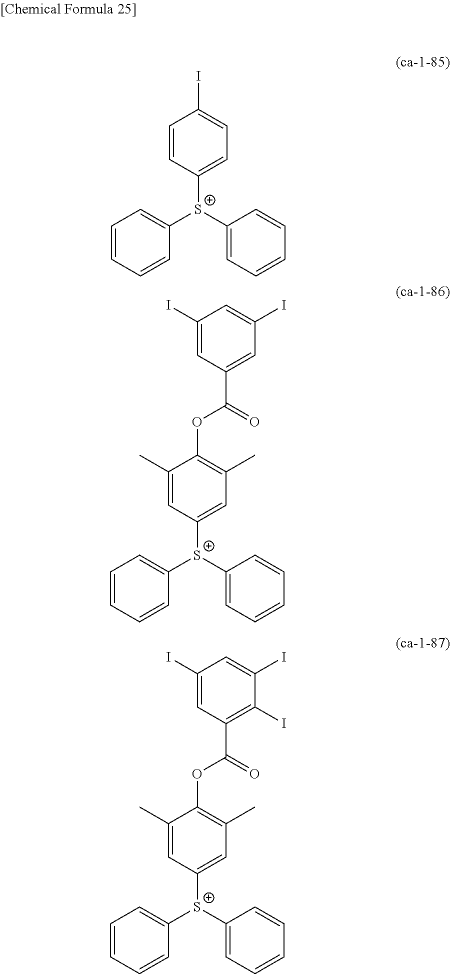 [SMILES: Ic1ccc([S+](c2ccccc2)c2ccccc2)cc1 ; Cc1cc([S+](c2ccccc2)c2ccccc2)cc(C)c1OC(=O)c1cc(I)cc(I)c1 ; Cc1cc([S+](c2ccccc2)c2ccccc2)cc(C)c1OC(=O)c1cc(I)cc(I)c1I]

Specific examples of suitable cations represented by Formula (ca-2) include a diphenyliodonium cation and a bis(4-tert-butylphenyl)iodonium cation.

Specific examples of suitable cations represented by Formula (ca-3) include cations each represented by Formulae (ca-3-1) to (ca-3-6).

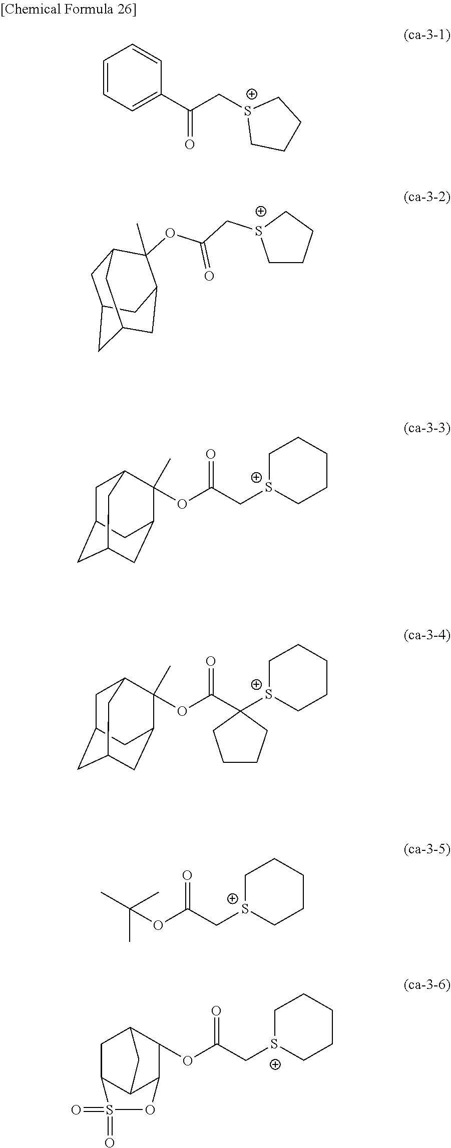 [SMILES: O=C(C[S+]1CCCC1)c1ccccc1 ; CC1(OC(=O)C[S+]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=C(C[S+]1CCCCC1)OC1C2CC3C1OS(=O)(=O)C3C2]

As the cation moiety ((M^{m+})_{1/m}) in Formula (a01-b) and Formula (a01-b0), cations each represented by Formulae (ca-1) to (ca-3) are more preferable, and cations each represented by Formulae (ca-1-1) to (ca-1-87), a diphenyliodonium cation, and a bis(4-tert-butylphenyl) iodonium cation are particularly preferable.

Among these, a cation represented by Formula (ca-1) is preferable, and from the viewpoint of high sensitivity, as a suitable cation represented by Formula (ca-1), a cation containing an electron-withdrawing group such as a fluorine atom, a fluorinated alkyl group, or a sulfonyl group as a substituent or a cation having an iodine atom is preferable, and a cation selected from the group consisting of cations each represented by Chemical Formulae (ca-1-44), (ca-1-71) to (ca-1-84), and (ca-1-85) to (ca-1-87) is particularly preferable.

The number of substituents may be, for example, 1 to 4, 1 to 3, 2, or 1.

Specific examples of the constitutional unit (a01) are shown below, but the examples are not limited thereto.

In the formulae shown below, R″ represents a hydrogen atom, a methyl group, or a trifluoromethyl group.

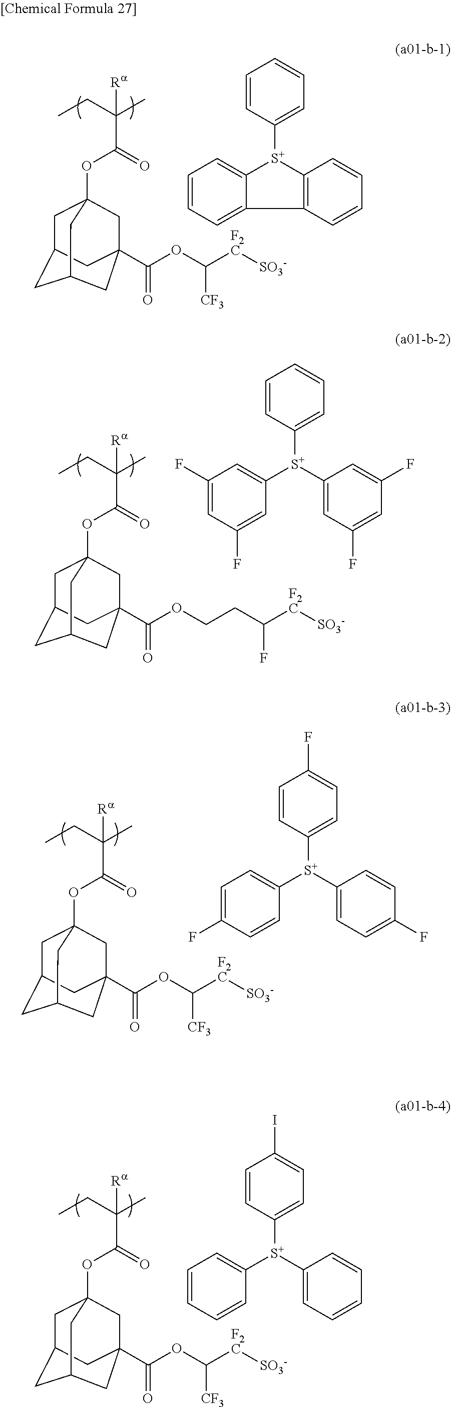

The constitutional unit (a01) is preferably a constitutional unit selected from the group consisting of constitutional units represented by any of Formulae (a01-b-1) to (a01-b-4).

The constitutional unit (a01) contained in the component (A1) may be used alone or two or more kinds thereof.

The proportion of the constitutional unit (a01) in the component (A1) is preferably in a range of 1% to 40% by mole, more preferably in a range of 2% to 30% by mole, still more preferably in a range of 5% to 25% by mole, and particularly preferably in a range of 10% to 20% by mole with respect to the total amount (100% by mole) of all constitutional units constituting the component (A1).

In a case where the proportion of the constitutional unit (a01) is greater than or equal to the lower limits of the above-described preferable ranges, the sensitivity in a case of forming a resist pattern is further improved, and the lithography characteristics such as reduction of roughness in a pattern are likely to be improved. In a case where the proportion of the constitutional unit (a01) is less than or equal to the upper limits of the above-described preferable ranges, the constitutional unit (a01) and other constitutional units are likely to be balanced.

<<Constitutional Unit (a02)>>

The constitutional unit (a02) is a constitutional unit having an onium salt structure that generates a carboxylic acid upon light exposure.

Examples of the constitutional unit (a02) include a constitutional unit (anion-bound unit) containing an anion group (—COO—) which generates a carboxylic acid upon light exposure in a side chain and a constitutional unit (cation-bound unit) containing a cation group (onium cation) which is decomposed upon light exposure in a side chain. Among these, in the resist pattern formation, from the viewpoint that satisfactory lithography characteristics are likely to be obtained, the constitutional unit (a02) is preferably a constitutional unit (anion-bound unit) containing an anion group (—COO—) which generates a carboxylic acid upon light exposure in a side chain.

Suitable examples of the constitutional unit (a02) include constitutional units derived from compounds represented by General Formula (a02-d).

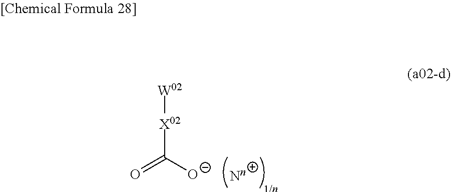

[In Formula (a02-d), W⁰² represents a polymerizable group-containing group. X⁰² represents a divalent linking group or a single bond. N^{m+} represents an n-valent onium cation. n represents an integer of 1 or greater.]

### {Anion Moiety}

In Formula (a02-d), the polymerizable group-containing group as W⁰² is the same as the description of the polymerizable group-containing group as W⁰¹ in Formula (a01-b) described above.

Suitable examples of the polymerizable group-containing group as W⁰² include a group represented by Formula: C(R^{X11})(R^{X12})═C(R^{X13})-Ya^{x0}-. In the formula, R^{X11}, R^{X12}, and R^{X13} each independently represent an alkyl group having 1 to 5 carbon atoms, a halogenated alkyl group having 1 to 5 carbon atoms, a halogen atom, or a hydrogen atom, and Ya^{x0} represents a divalent linking group or a single bond.

It is preferable that R^{X11}, R^{X12}, and R^{X13} represent a hydrogen atom, an alkyl group having 1 to 5 carbon atoms, or a fluorinated alkyl group having 1 to 5 carbon atoms and particularly preferable that R^{X11} and R^{X12} represent a hydrogen atom and R^{X13} represents a hydrogen atom or a methyl group from the viewpoint of industrial availability.

Examples of Ya^{x0} include a single bond, an ester bond (—C(═O)—O—), an oxycarbonyl group (—O—C(═O)—), an ether bond (—O—), a linear or branched alkylene group, and arylene group which may have a substituent, and an optional combination thereof.

In Formula (a02-d), examples of the divalent linking group represented by X⁰² include a divalent hydrocarbon group which may have a substituent and a divalent linking group having a heteroatom.

Each of the divalent hydrocarbon group which may have a substituent and the divalent linking group having a heteroatom as X⁰² is the same as the description of the divalent hydrocarbon group which may have a substituent and the divalent linking group having a heteroatom as X⁰¹ in Formula (a01-b).

X⁰² represents preferably a cyclic aliphatic hydrocarbon group which may have a substituent, an aromatic hydrocarbon group which may have a substituent, an ester bond [—C(═O)—O— or —O—C(═O)—], an ether bond (—O—), an amide bond [—NH—C(═O)— or —C(═O)—NH—], a linear or branched aliphatic hydrocarbon group, a combination of two or more of these groups, or a single bond and more preferably an aromatic hydrocarbon group which may have a substituent, an ether bond (—O—), a linear or branched aliphatic hydrocarbon group, or a combination of two or more of these groups.

Suitable examples of the constitutional unit (a02) include a constitutional unit represented by General Formula (a02-d0).

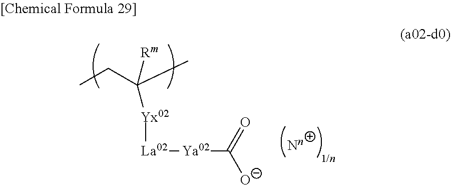

[In the formula, R^{m} represents an alkyl group having 1 to 5 carbon atoms, a halogenated alkyl group having 1 to 5 carbon atoms, a halogen atom, or a hydrogen atom. Yx⁰² represents a divalent linking group or a single bond. La⁰² represents a hydrocarbon group which may have a substituent. Ya⁰² represents a divalent linking group or a single bond. N^{m+} represents an n-valent onium cation. n represents an integer of 1 or greater.]

In Formula (a02-d0), R^{m} has the same definition as that for R^{m} in Formula (a01-b0). It is preferable that R^{m} represents a hydrogen atom or a methyl group from the viewpoint of the industrial availability.

In Formula (a02-d0), Yx⁰² has the same definition as that for Yx⁰¹ in Formula (a01-b0). Specific examples of Yx⁰² include an ester bond (—C(═O)—O—), an oxycarbonyl group (—O—C(═O)—), an ether bond (—O—), a linear or branched alkylene group, an arylene group which may have a substituent, an optional combination of these groups, and a single bond.

Among these, a combination of an arylene group which may have a substituent, a linear or branched alkylene group, and an ether bond (—O—), an ester bond (—C(═O)—O—), or a single bond is preferable.

In Formula (a02-d0), La⁰² is the same as the description of the hydrocarbon group which may have a substituent as La⁰¹ in Formula (a01-b0).

Examples of La⁰² include the same groups as those for the aliphatic hydrocarbon group (the chain-like aliphatic hydrocarbon group or the aliphatic hydrocarbon group having a ring in the structure), and the aromatic hydrocarbon group described in the section of the divalent hydrocarbon group which may have a substituent as X⁰¹. Among these, an aliphatic hydrocarbon group having a ring in the structure or an aromatic hydrocarbon group is preferable, and a cyclic aliphatic hydrocarbon group (group obtained by removing two hydrogen atoms from an aliphatic hydrocarbon ring) which may have a substituent, and an aromatic hydrocarbon group which may have a substituent are more preferable.

The cyclic aliphatic hydrocarbon group as La⁰² may be a monocyclic alicyclic hydrocarbon group or a polycyclic alicyclic hydrocarbon group, and is preferably a polycyclic alicyclic hydrocarbon group, more preferably a group obtained by removing two hydrogen atoms from a polycycloalkane, and still more preferably a group obtained by removing two hydrogen atoms from adamantane.

The cyclic aliphatic hydrocarbon group may or may not have a substituent. Examples of the substituent include an alkyl group, an alkoxy group, a halogen atom, a halogenated alkyl group, a hydroxy group, and a carbonyl group.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂—O— is preferable.

As the aromatic hydrocarbon group as La⁰², a group (an arylene group or a heteroarylene group) obtained by removing two hydrogen atoms from an aromatic hydrocarbon ring or an aromatic heterocyclic ring is preferable, an arylene group is more preferable, and a phenylene group is still more preferable.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of the substituent include an alkyl group, an alkoxy group, a halogen atom, a halogenated alkyl group, and a hydroxy group.

Among these, La⁰² represents preferably an aromatic hydrocarbon group which may have a substituent and more preferably an arylene group which may have a substituent.

From the viewpoint of the solubility in the developing solution, a hydroxy group is more preferable as the substituent that the aromatic hydrocarbon group as La⁰² may have. The number of hydroxy groups as a substituent is, for example, 2 or 1. In addition, a halogen atom or a hydroxy group is preferable as the substituent that the aromatic hydrocarbon group as La⁰² may have. From the viewpoint of increasing the sensitivity in a case of forming a resist pattern, a halogen atom is more preferable, and an iodine atom is still more preferable. The number of iodine atoms as the substituent is, for example, 1 to 4, 1 to 3, 2, or 1. As the number of iodine atoms as the substituent increases, the sensitivity is likely to increase. In addition, due to the effect of the iodine atoms, reduction of pattern bridging in regions with low exposure amounts and reduction of pattern collapse in regions with high exposure amounts are achieved, and thus the process margin is likely to be widened.

In Formula (a02-d0), examples of the divalent linking group represented by Ya⁰² include the same groups as those for the divalent linking group having a heteroatom as X⁰¹ in Formula (a01-b). Examples of Ya⁰² include an oxygen atom (ether bond: —O—), an ester bond (—C(═O)—O—), an oxycarbonyl group (—O—C(═O)—), a linear or branched alkylene group, an optional combination thereof, and a single bond. Among these, a single bond is preferable.

Specific examples of the constitutional unit (a02) are shown below, but the examples are not limited thereto.

In each formula, R^{α} represents a trifluoromethyl group, a methyl group, or a hydrogen atom. N^{m+} represents an n-valent onium cation. n represents an integer of 1 or more.

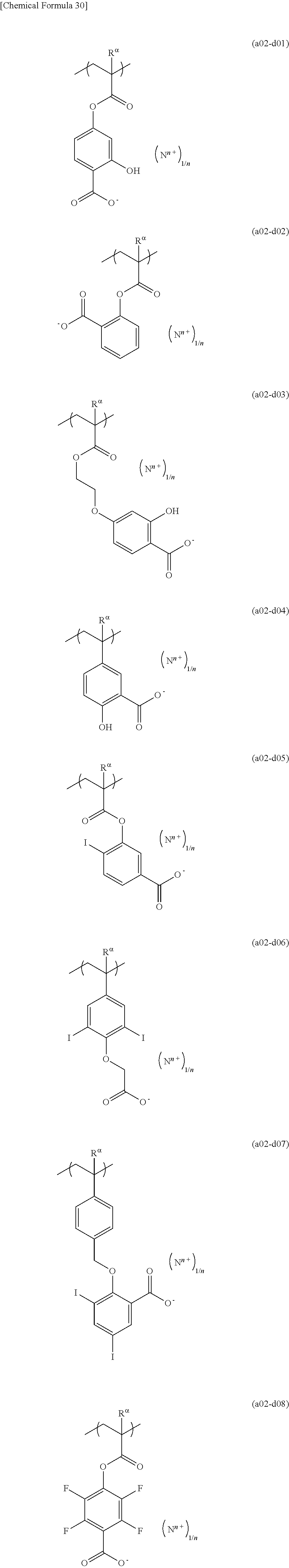

A constitutional unit selected from the group consisting of constitutional units represented by any of Formulae (a02-d01) to (a02-d08) is preferable, and a constitutional unit selected from the group consisting of constitutional units represented by any of Formulae (a02-d01) to (a02-d04) is more preferable.

### {Cation Moiety}

In Formula (a02-d) and Formula (a02-d0), N^{m+} represents an n-valent onium cation. n represents an integer of 1 or more. Among the onium cations as Nⁿ⁺, a sulfonium cation or an iodonium cation is preferable, and a sulfonium cation is more preferable.

Preferred examples of the cation moiety (N^{m+})_{1/n} include onium cations each represented by General Formulae (ca-1) to (ca-3). Among these, the cation represented by any of Formulae (ca-1-1) to (ca-1-87), a diphenyliodonium cation, or a bis(4-tert-butylphenyl) iodonium cation is more preferable.

Among these, a cation represented by Formula (ca-1) is preferable, and from the viewpoint of high sensitivity, as a suitable cation represented by Formula (ca-1), a cation containing an electron-withdrawing group such as a fluorine atom, a fluorinated alkyl group, or a sulfonyl group as a substituent or a cation having an iodine atom is preferable, and a cation selected from the group consisting of cations each represented by Chemical Formulae (ca-1-44), (ca-1-71) to (ca-1-84), and (ca-1-85) to (ca-1-87) is particularly preferable.

The number of substituents may be, for example, 1 to 4, 1 to 3, 2, or 1.

Specific examples of the constitutional unit (a02) are shown below, but the examples are not limited thereto.

In the formulae shown below, R^{α} represents a hydrogen atom, a methyl group, or a trifluoromethyl group.

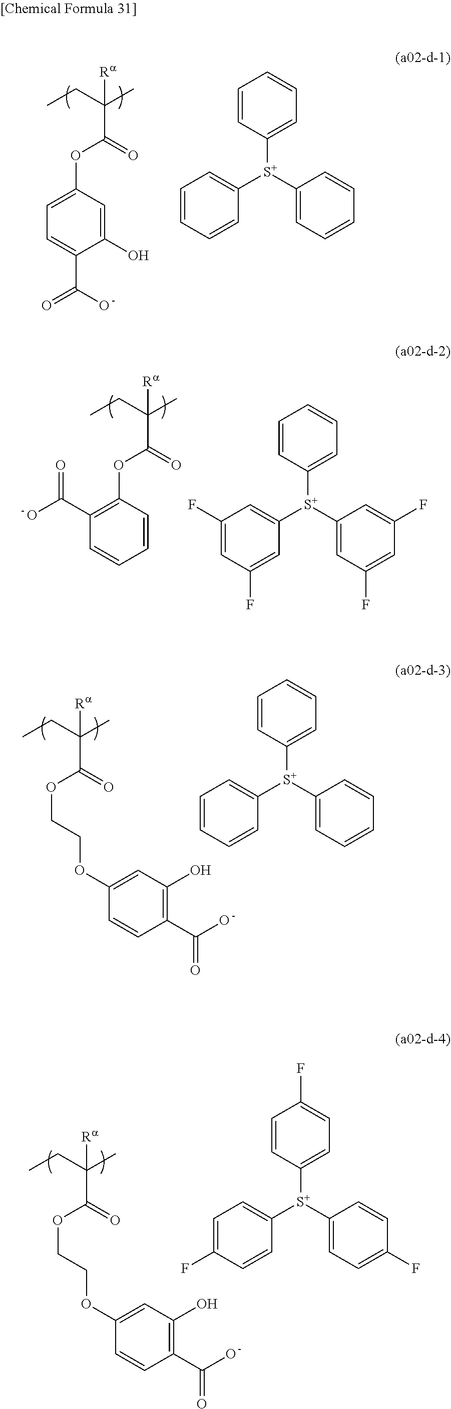

The constitutional unit (a02) is preferably a constitutional unit selected from the group consisting of constitutional units each represented by Formulae (a02-d-1) to (a02-d-4).

The constitutional unit (a02) that the component (A1) has may be used alone or two or more kinds thereof.

The proportion of the constitutional unit (a02) in the component (A1) is preferably in a range of 0.5% to 20% by mole, more preferably in a range of 1% to 15% by mole, still more preferably in a range of 1% to 10% by mole, and particularly preferably in a range of 2% to 8% by mole with respect to the total amount (100% by mole) of all constitutional units constituting the component (A1).

In a case where the proportion of the constitutional unit (a02) is greater than or equal to the lower limits of the above-described preferable ranges, the lithography characteristics such as reduction of roughness in a pattern are likely to be improved. In a case where the proportion of the constitutional unit (a02) is less than or equal to the upper limits of the above-described preferable ranges, the constitutional unit (a02) and other constitutional units are likely to be balanced.

### <<Other Constitutional Units>>

The component (A1) may have other constitutional units as necessary in addition to the constitutional unit (a01) and the constitutional unit (a02) described above. Examples of the other constitutional units include a constitutional unit (a1) containing an acid decomposable group whose polarity is increased by the action of an acid; a constitutional unit (a10) represented by General Formula (a10-1); a constitutional unit (a2) containing a lactone-containing cyclic group; and a constitutional unit (a8) derived from a compound represented by General Formula (a8-1).

### Constitutional Unit (a1):

The constitutional unit (a1) is a constitutional unit that contains an acid decomposable group whose polarity is increased by the action of an acid. Examples of the acid dissociable group constituting an acid decomposable group include the same groups as those which have been suggested as acid dissociable groups of the base resin for a chemically amplified resist composition.

Examples of the acid dissociable group of the base resin for a chemically amplified resist composition, which has been suggested, include “acetal type acid dissociable groups”, “tertiary alkyl ester type acid dissociable groups”, “tertiary alkyl oxycarbonyl acid dissociable groups”, and “secondary alkyl ester type acid dissociable groups”.

### Acetal Type Acid Dissociable Group:

Examples of the acid dissociable group that protects a carboxy group or a hydroxyl group in the polar groups include an acid dissociable group represented by General Formula (a1-r-1) (hereinafter, also referred to as “acetal type acid dissociable group”).

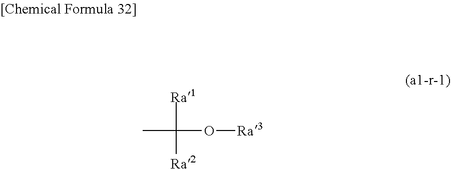

[In the formula, Ra′¹ and Ra′² represent a hydrogen atom or an alkyl group. Ra′³ represents a hydrocarbon group, and Ra′³ may be bonded to any of Ra′¹ and Ra′² to form a ring.]

In Formula (a1-r-1), it is preferable that at least one of Ra′¹ and Ra′² represents a hydrogen atom and more preferable that both Ra′¹ and Ra′² represent a hydrogen atom. In a case where Ra′¹ or Ra′² represents an alkyl group, examples of the alkyl group include the same alkyl groups exemplified as the substituent which may be bonded to the carbon atom at the α-position in the description on α-substituted acrylic acid ester. Among these, an alkyl group having 1 to 5 carbon atoms is preferable. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In Formula (a1-r-1), examples of the hydrocarbon group as Ra′³ include a linear or branched alkyl group and a cyclic hydrocarbon group.

The linear alkyl group has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group has preferably 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′³ represents a cyclic hydrocarbon group, the hydrocarbon group may be an alicyclic hydrocarbon group or an aromatic hydrocarbon group and may be a polycyclic group or a monocyclic group.

The alicyclic hydrocarbon group which is a monocyclic group is preferably a group in which one hydrogen atom has been removed from a monocycloalkane. The monocycloalkane has preferably 3 to 6 carbon atoms, and specific examples thereof include cyclopentane and cyclohexane.

The alicyclic hydrocarbon group which is a polycyclic group is preferably a group in which one hydrogen atom has been removed from a polycycloalkane. The polycycloalkane preferably has 7 to 12 carbon atoms, and specific examples thereof include adamantane, norbornane, isobornane, tricyclodecane, and tetracyclododecane.

In a case where the cyclic hydrocarbon group as Ra′³ becomes an aromatic hydrocarbon group, the aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms.

Specifically, as the aromatic ring, an aromatic hydrocarbon ring such as benzene, naphthalene, anthracene, and phenanthrene; and an aromatic heterocyclic ring in which some carbon atoms constituting the aromatic hydrocarbon ring have been substituted with heteroatoms are exemplary examples. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group in which one hydrogen atom has been removed from the above-described aromatic hydrocarbon ring or aromatic heterocyclic ring (such as an aryl group or a heteroaryl group); a group in which one hydrogen atom has been removed from an aromatic compound having two or more aromatic rings (such as biphenyl or fluorene); and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aromatic hydrocarbon ring or aromatic heterocyclic ring is preferably in a range of 1 to 4, more preferably 1 or 2, and particularly preferably 1.

The cyclic hydrocarbon group as Ra′³ may include a substituent. Examples of the substituent include —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{P2}—CN, and —R^{P2}—COOH (hereinafter, these substituents will also be collectively referred to as “Ra^{x5}”). Here, R^{P1} represents a chain-like monovalent saturated hydrocarbon group having 1 to 10 carbon atoms, a monovalent aliphatic cyclic saturated hydrocarbon group having 3 to 20 carbon atoms, or a monovalent aromatic hydrocarbon group having 6 to 30 carbon atoms. Further, R^{P2} represents a single bond, a chain-like divalent saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. Some or all hydrogen atoms in the chain-like saturated hydrocarbon group, the aliphatic cyclic saturated hydrocarbon group, and the aromatic hydrocarbon group as R^{P1} and R^{P2} may be substituted with fluorine atoms. The aliphatic cyclic hydrocarbon group may have one or more of one kind of substituents or one or more of each of plural kinds of the substituents.

Examples of the chain-like monovalent saturated hydrocarbon group having 1 to 10 carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, or an adamantyl group.

Examples of the monovalent aromatic hydrocarbon group having 6 to 30 carbon atoms include a group formed by removing one hydrogen atom from an aromatic hydrocarbon ring such as benzene, biphenyl, fluorene, naphthalene, anthracene, or phenanthrene.

In a case where Ra′³ is bonded to any of Ra′¹ and Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

### Tertiary Alkyl Ester Type Acid Dissociable Group:

Examples of the acid dissociable group that protects a carboxy group among the polar groups include an acid dissociable group represented by General Formula (a1-r-2).

Among the examples of the acid dissociable group represented by Formula (a1-r-2), a group formed of an alkyl group is referred to as “tertiary alkyl ester type acid dissociable group” for convenience.

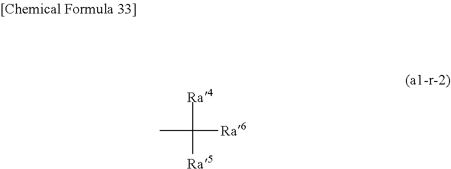

[In the formula, Ra′⁴ to Ra′⁶ each independently represent a hydrocarbon group, and Ra′⁵ and Ra′⁶ may be bonded to each other to form a ring.]

Examples of the hydrocarbon group as Ra′⁴ include a linear or branched alkyl group, a chain-like or cyclic alkenyl group, a chain-like alkynyl group, and a cyclic hydrocarbon group.

Examples of the linear or branched alkyl group and the cyclic hydrocarbon group (an alicyclic hydrocarbon group which is a monocyclic group, an alicyclic hydrocarbon group which is a polycyclic group, or an aromatic hydrocarbon group) as Ra′⁴ include the same groups as those for Ra′³.

As the chain-like or cyclic alkenyl group as Ra′⁴, an alkenyl group having 2 to carbon atoms is preferable.

Examples of the hydrocarbon group as Ra′⁵ or Ra′⁶ include the same groups as those for Ra′³.

In a case where Ra′⁵ and Ra′⁶ are bonded to each other to form a ring, suitable examples thereof include a group represented by Formula (a1-r2-1), a group represented by Formula (a1-r2-2), and a group represented by Formula (a1-r2-3).

Meanwhile, in a case where Ra′⁴ to Ra′⁶ represent an independent hydrocarbon group without being bonded to one another, suitable examples thereof include a group represented by Formula (a1-r2-4).

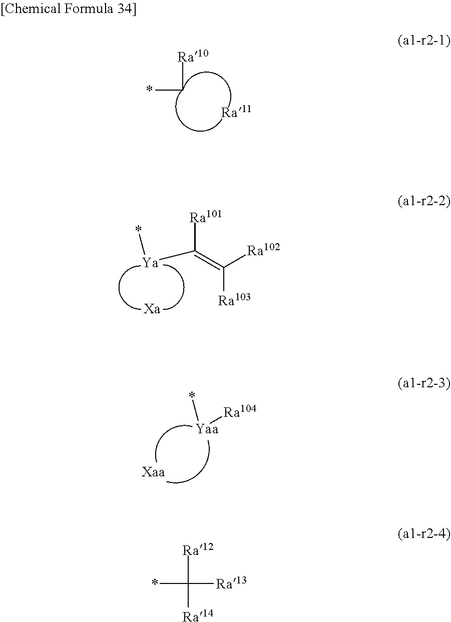

[In Formula (a1-r2-1), Ra′¹⁰ represents a linear or branched alkyl group having 1 to 12 carbon atoms, in which a part thereof may be substituted with a halogen atom or a heteroatom-containing group. Ra′¹¹ represents a group that forms an aliphatic cyclic group with the carbon atom to which Ra′¹⁰ has been bonded. In Formula (a1-r2-2), Ya represents a carbon atom. Xa represents a group that forms a cyclic hydrocarbon group with Ya. Some or all hydrogen atoms in this cyclic hydrocarbon group may be substituted. Ra¹⁰¹ to Ra¹⁰³ each independently represent a hydrogen atom, a chain-like monovalent saturated hydrocarbon group having 1 to 10 carbon atoms, or a monovalent aliphatic cyclic saturated hydrocarbon group having 3 to 20 carbon atoms. Some or all hydrogen atoms in the chain-like saturated hydrocarbon group and the aliphatic cyclic saturated hydrocarbon group may be substituted. Two or more of Ra¹⁰¹ to Ra¹⁰³ may be bonded to one another to form a cyclic structure. In Formula (a1-r2-3), Yaa represents a carbon atom. Xaa represents a group that forms an aliphatic cyclic group with Yaa. Ra¹⁰⁴ represents an aromatic hydrocarbon group which may have a substituent. In Formula (a1-r2-4), Ra′¹² and Ra′¹³ each independently represent a monovalent chain-like saturated hydrocarbon group having 1 to 10 carbon atoms. Some or all of hydrogen atoms contained in the chain-like saturated hydrocarbon group may be substituted. Ra′¹⁴ represents a hydrocarbon group which may have a substituent. * represents a bonding site (the same applies hereinafter).]

In Formula (a1-r2-1), Ra′¹⁰ represents a linear or branched alkyl group having 1 to 12 carbon atoms, in which a part thereof may be substituted with a halogen atom or a heteroatom-containing group.

The linear alkyl group as Ra′¹⁰ has 1 to 12 carbon atoms, preferably 1 to 10 carbon atoms, and particularly preferably 1 to 5 carbon atoms.

Examples of the branched alkyl group as Ra′¹⁰ include those for Ra′³ described above.

The alkyl group in Ra′¹⁰ may be partially substituted with a halogen atom or a heteroatom-containing group. For example, some hydrogen atoms constituting the alkyl group may be substituted with a halogen atom or a heteroatom-containing group. Further, some carbon atoms (methylene group or the like) constituting the alkyl group may be substituted with a heteroatom-containing group.

Examples of the heteroatoms here include an oxygen atom, a nitrogen atom, and a sulfur atom. Examples of the heteroatom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

In Formula (a1-r2-1), preferred examples of Ra′¹¹ (an aliphatic cyclic group that is formed together with a carbon atom to which Ra′¹⁰ is bonded) include the groups described as the alicyclic hydrocarbon group (alicyclic hydrocarbon group) which is a monocyclic group or a polycyclic group as Ra′³ in Formula (a1-r-1). Among these, it is preferably a monocyclic alicyclic hydrocarbon group, and specifically, it is more preferably a cyclopentyl group or a cyclohexyl group.

In Formula (a1-r2-2), examples of the cyclic hydrocarbon group that is formed by Xa together with Ya include a group in which one or more hydrogen atoms have been further removed from the cyclic monovalent hydrocarbon group (alicyclic hydrocarbon group) as Ra′³ in Formula (a1-r-1).

The cyclic hydrocarbon group that is formed by Xa together with Ya may have a substituent. Examples of the substituent include those which are the same as the substituents which may be included in the cyclic hydrocarbon group as Ra′³.

In Formula (a1-r2-2), examples of the chain-like monovalent saturated hydrocarbon group having 1 to 10 carbon atoms as Ra¹⁰¹ to Ra¹⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, or an adamantyl group.

From the viewpoint of ease of synthesis, Ra¹⁰¹ to Ra¹⁰³ represent preferably a hydrogen atom or a chain-like monovalent saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by Ra¹⁰¹ to Ra¹⁰³ are the same as those for Ra^{x5}.

Examples of the group having a carbon-carbon double bond generated by two or more of Ra¹⁰¹ to Ra¹⁰³ being bonded to one another to form a cyclic structure include a cyclopentenyl group, a cyclohexenyl group, a methylcyclopentenyl group, a methylcyclohexenyl group, a cyclopentylidenethenyl group, and a cyclohexylidenethenyl group. Among these, from the viewpoint of ease of synthesis, a cyclopentenyl group, a cyclohexenyl group, or a cyclopentylidenethenyl group is preferable.

In Formula (a1-r2-3), as the aliphatic cyclic group that is formed by Xaa together with Yaa, the group described as the alicyclic hydrocarbon group which is a monocyclic group or a polycyclic group as Ra′³ in Formula (a1-r-1) is preferable.

In Formula (a1-r2-3), examples of the aromatic hydrocarbon group as Ra¹⁰4 include a group in which one or more hydrogen atoms have been removed from an aromatic hydrocarbon ring having 5 to 30 carbon atoms. Among the examples, Ra¹⁰⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent which may be included in Ra¹⁰⁴ in Formula (a1-r2-3) include a methyl group, an ethyl group, a propyl group, a hydroxyl group, a carboxyl group, a halogen atom, an alkoxy group (such as a methoxy group, an ethoxy group, a propoxy group, or a butoxy group), and an alkyloxycarbonyl group.

In Formula (a1-r2-4), Ra′¹² and Ra′¹³ each independently represent a monovalent chain-like saturated hydrocarbon group having 1 to 10 carbon atoms. Examples of the monovalent chain-like saturated hydrocarbon group having 1 to 10 carbon atoms as Ra′¹² and Ra′¹³ include the same one as the monovalent chain-like saturated hydrocarbon group having 1 to 10 carbon atoms as Ra¹⁰¹ to Ra¹⁰³ as described above. Some or all of hydrogen atoms contained in the chain-like saturated hydrocarbon group may be substituted.

Ra′¹² and Ra′¹³ represent preferably an alkyl group having 1 to 5 carbon atoms, more preferably an alkyl group having 1 to 5 carbon atoms, still more preferably a methyl group or an ethyl group, and particularly preferably a methyl group.

In a case where the chain-like saturated hydrocarbon group represented by Ra′¹² and Ra′¹³ is substituted, examples of the substituent are those for Ra^{x5} described above.

In Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an alicyclic hydrocarbon group or an aromatic hydrocarbon group and may be a polycyclic group or a monocyclic group.

The alicyclic hydrocarbon group which is a monocyclic group is preferably a group in which one hydrogen atom has been removed from a monocycloalkane. The monocycloalkane has preferably 3 to 6 carbon atoms, and specific examples thereof include cyclopentane and cyclohexane.

The alicyclic hydrocarbon group which is a polycyclic group is preferably a group in which one hydrogen atom has been removed from a polycycloalkane. The polycycloalkane preferably has 7 to 12 carbon atoms, and specific examples thereof include adamantane, norbornane, isobornane, tricyclodecane, and tetracyclododecane.

Examples of the aromatic hydrocarbon group as Ra′¹⁴ include the same groups as those for the aromatic hydrocarbon group as Ra¹⁰⁴. Among these, Ra′¹⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent which may be included in Ra′¹⁴ include the same groups as those for the substituent which may be included in Ra¹⁰⁴.

In a case where Ra′¹⁴ in Formula (a1-r2-4) represents a naphthyl group, the position bonded to the tertiary carbon atom in Formula (a1-r2-4) may be the 1-position or the 2-position of the naphthyl group.

In a case where Ra′¹⁴ in Formula (a1-r2-4) represents an anthryl group, the position bonded to the tertiary carbon atom in Formula (a1-r2-4) may be the 1-position, the 2-position, or the 9-position of the anthryl group.

Specific examples of the group represented by Formula (a1-r2-1) are shown below.

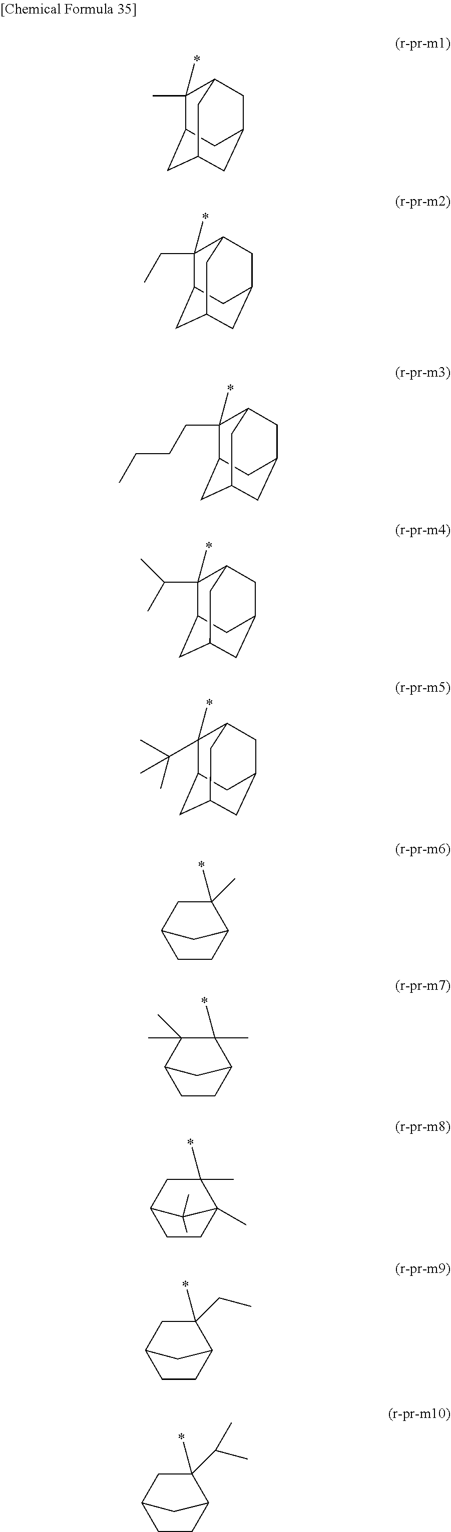

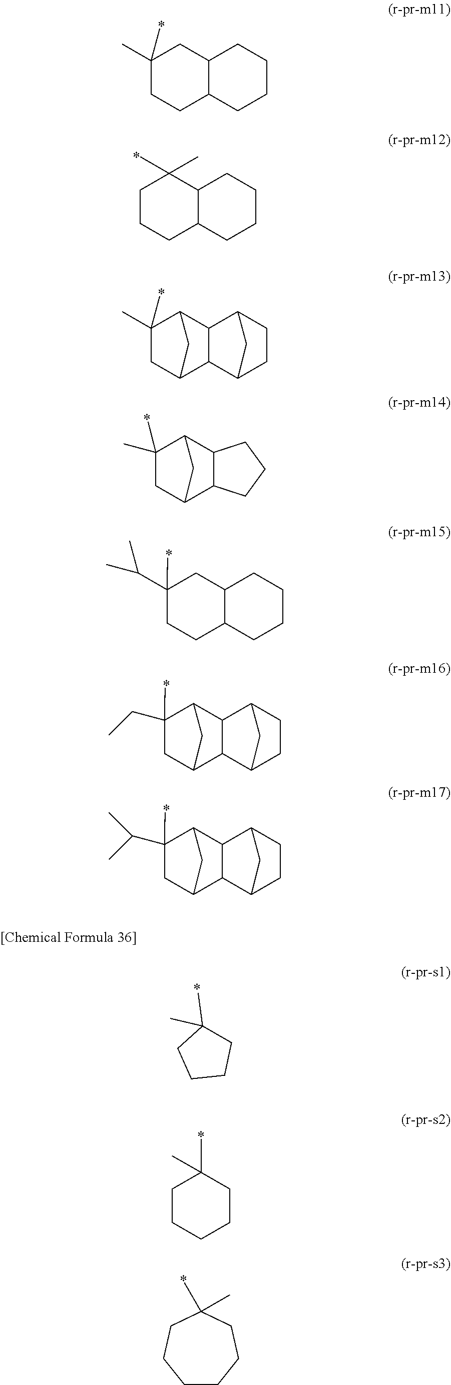

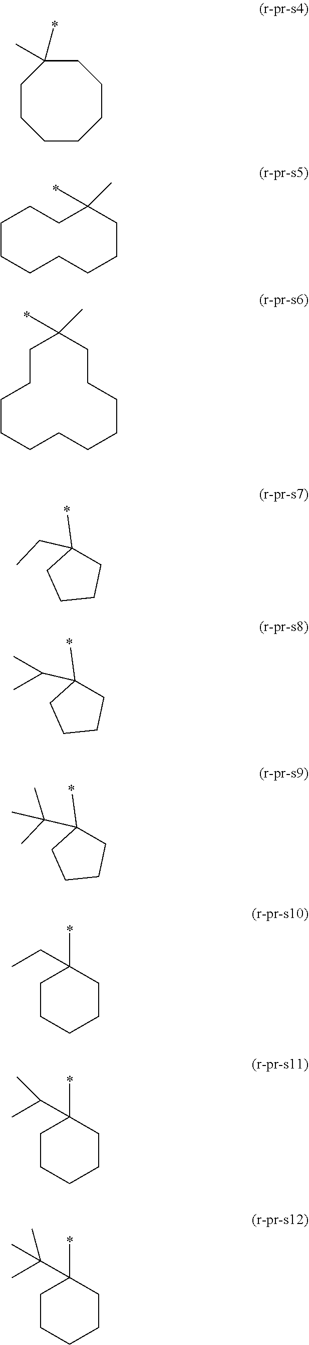

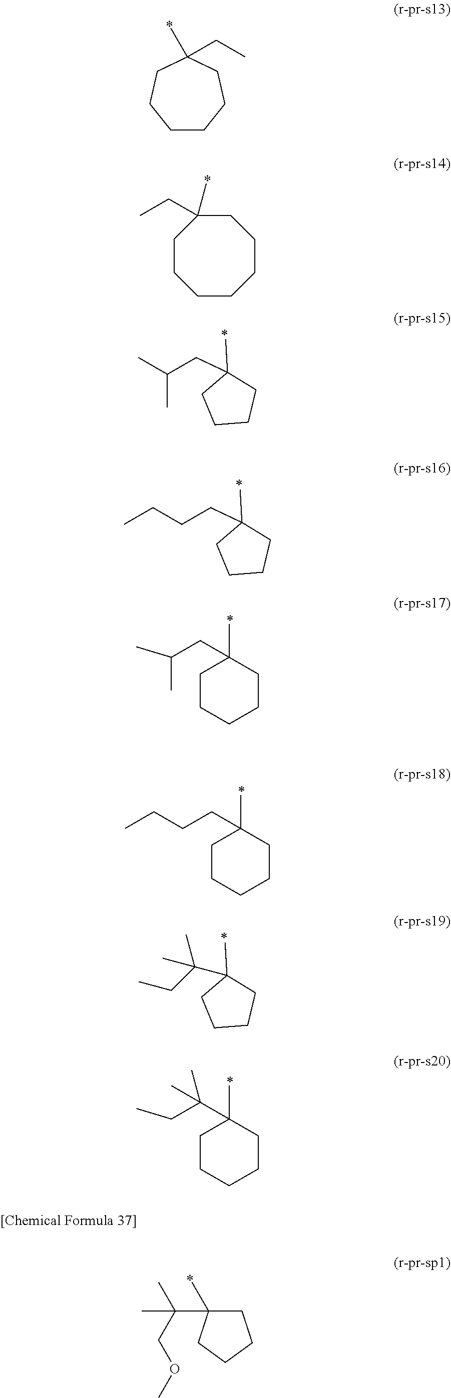

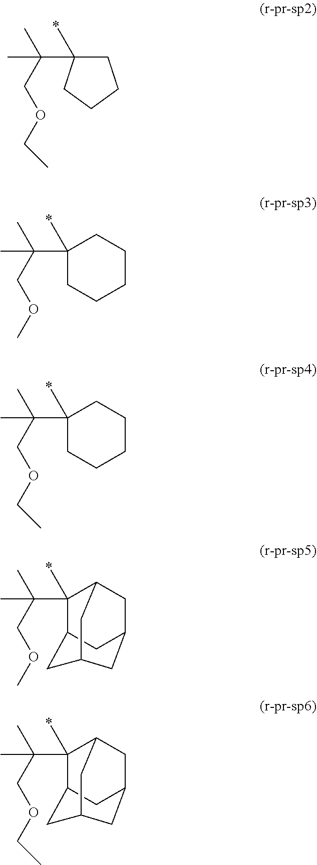

Specific examples of the group represented by Formula (a1-r2-2) are shown below.

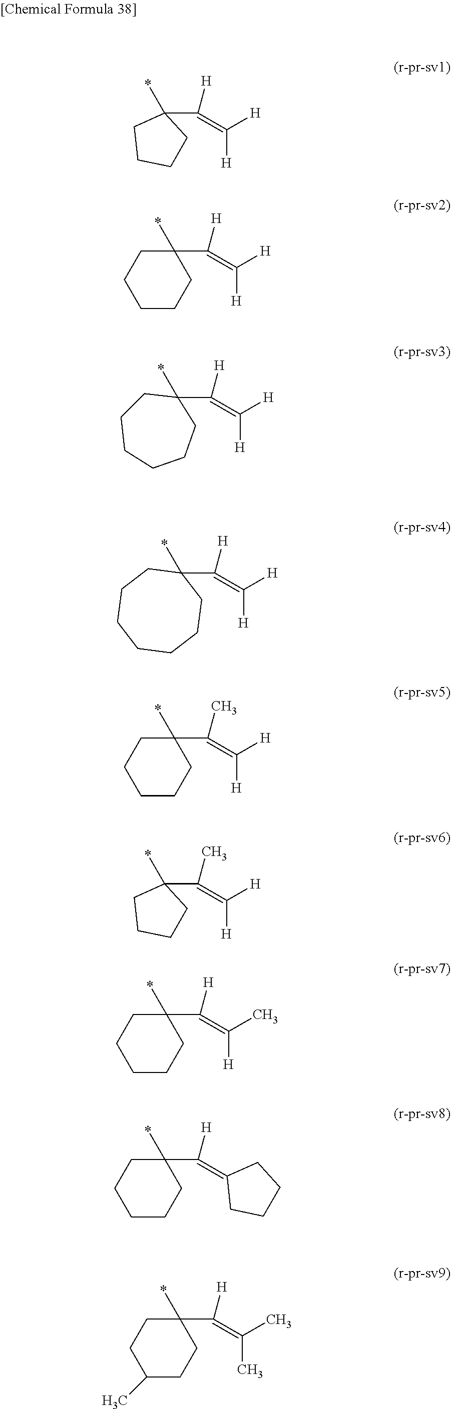

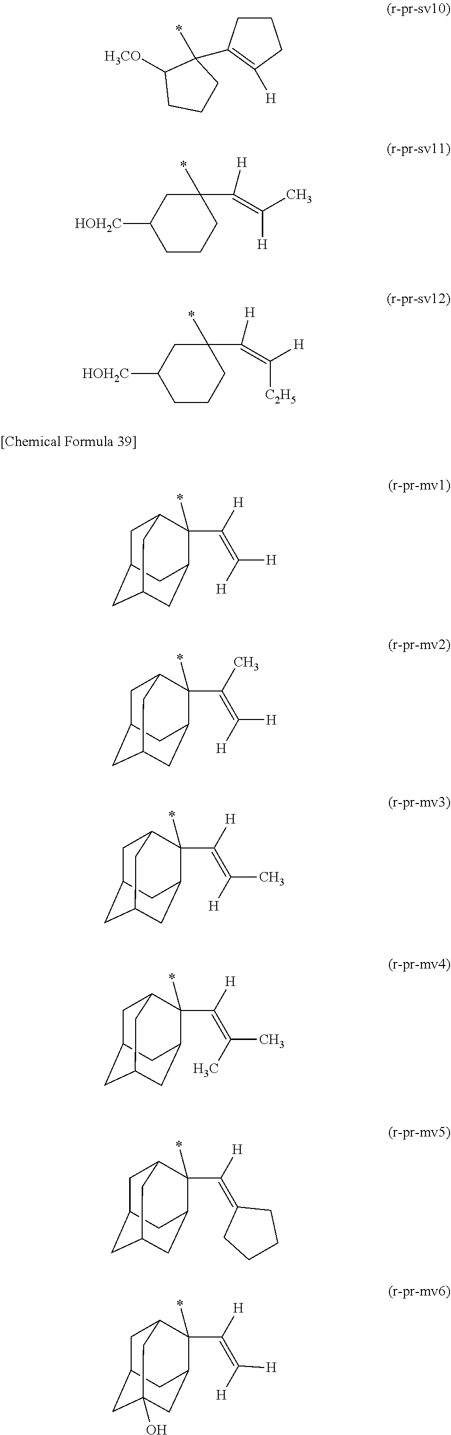

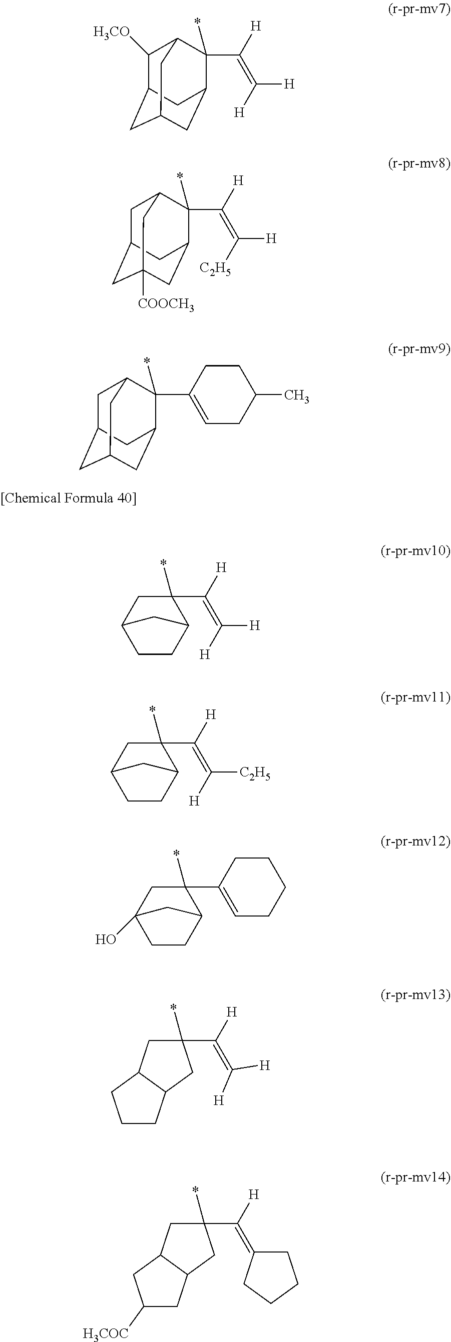

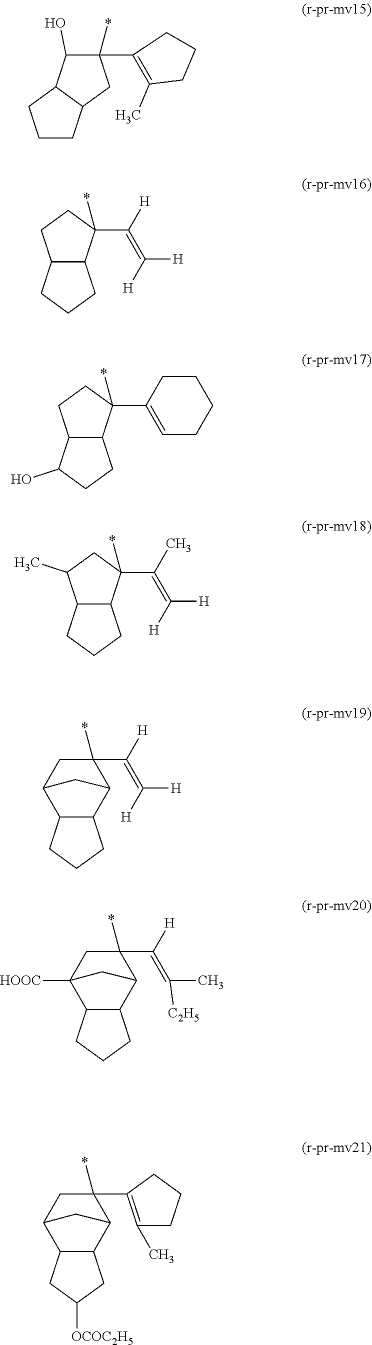

Specific examples of the group represented by Formula (a1-r2-3) are shown below.

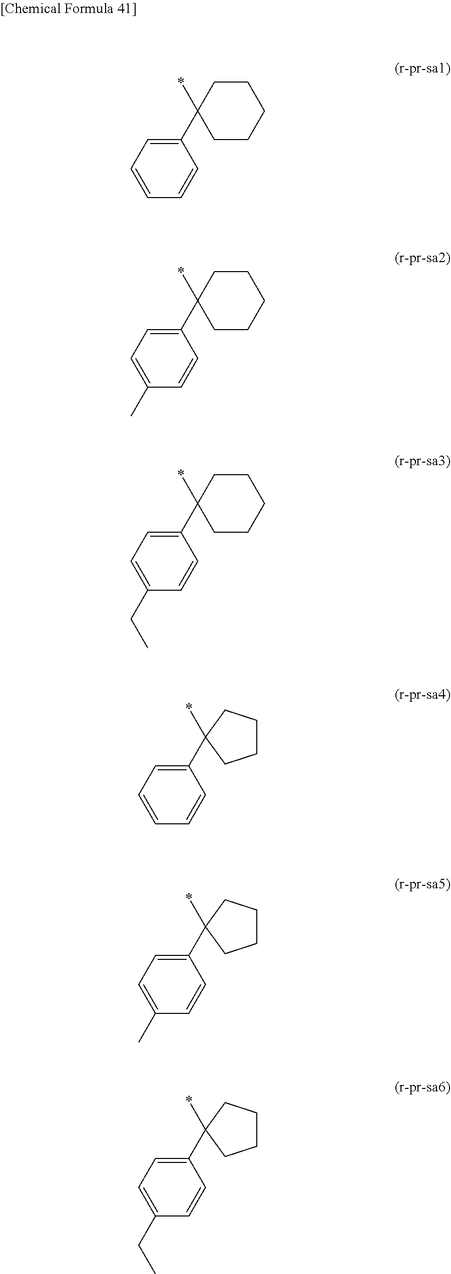 [SMILES: *C1(c2ccccc2)CCCCC1 ; *C1(c2ccc(C)cc2)CCCCC1 ; *C1(c2ccc(CC)cc2)CCCCC1 ; *C1(c2ccccc2)CCCC1 ; *C1(c2ccc(C)cc2)CCCC1 ; *C1(c2ccc(CC)cc2)CCCC1]

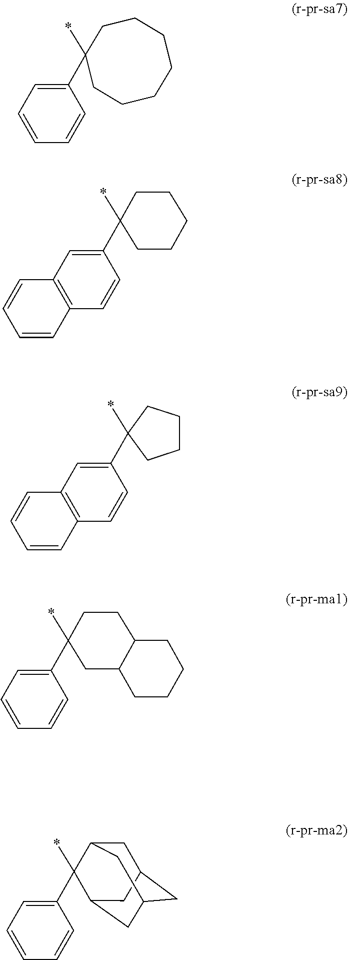 [SMILES: *C1(c2ccccc2)CCCCCCC1 ; *C1(c2ccc3ccccc3c2)CCCCC1 ; *C1(c2ccc3ccccc3c2)CCCC1 ; *C1(c2ccccc2)CCC2CCCCC2C1 ; *C1(c2ccccc2)C2CC3CC(C2)CC1C3]

Specific examples of the group represented by Formula (a1-r2-4) are shown below.

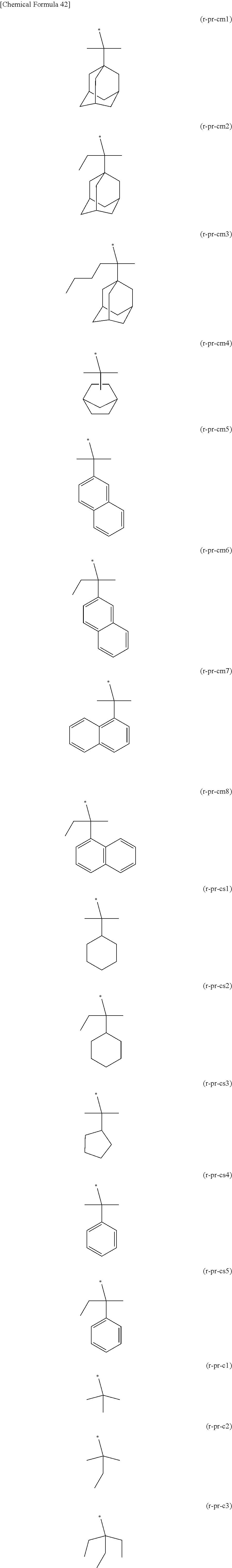

### Tertiary Alkyloxycarbonyl Acid Dissociable Group:

Examples of the acid dissociable group that protects a hydroxyl group among the polar groups include an acid dissociable group (hereinafter, also referred to as “tertiary alkyloxycarbonyl acid dissociable group” for convenience) represented by General Formula (a1-r-3).

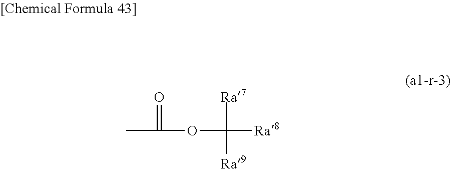

[In the formula, Ra′⁷ to Ra′⁹ each independently represent an alkyl group.]

In Formula (a1-r-3), Ra′⁷ to Ra′⁹ each represent preferably an alkyl group having 1 to 5 carbon atoms and more preferably an alkyl group having 1 to 3 carbon atoms.

Further, the total number of carbon atoms in each alkyl group is preferably in a range of 3 to 7, more preferably in a range of 3 to 5, and most preferably 3 or 4.

### Secondary Alkyl Ester Type Acid Dissociable Group:

Examples of the acid dissociable group that protects a carboxy group among the polar groups include an acid dissociable group represented by General Formula (a1-r-4).

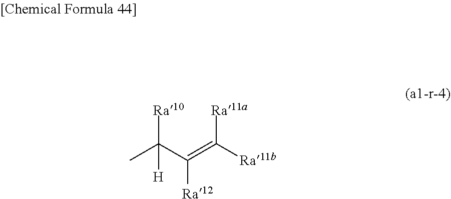

[In the formula, Ra′¹⁰ represents a hydrocarbon group. Ra′^{11a} and Ra′^{11b} each independently represent a hydrogen atom, a halogen atom, or an alkyl group. Ra′¹² represents a hydrogen atom or a hydrocarbon group. Ra′¹⁰ and Ra′^{11a} or Ra′^{11b} may be bonded to each other to form a ring. Ra′^{11a} or Ra′^{11b} and Ra′¹² may be bonded to each other to form a ring.]

Examples of the hydrocarbon group as Ra′¹⁰ or Ra′¹² in the formula include the same groups as those for Ra′³.

Examples of the alkyl group as Ra′^{11a} and Ra′^{11b} in the formula include the same groups as those for the alkyl group as Ra′¹.

In the formula, the hydrocarbon group as Ra′¹⁰ or Ra′¹² and the alkyl group as Ra′^{11a} and Ra′^{11b} may have a substituent. Examples of this substituent include Ra^{x5} described above.

Ra′¹⁰ and Ra′^{1a} or Ra′^{11b} may be bonded to each other to form a ring. The ring may be a polycyclic ring or a monocyclic ring, and may be an alicyclic ring or an aromatic ring.

The alicyclic ring and the aromatic ring may have a heteroatom.

Among the examples described above, as the ring formed by Ra′¹⁰ and Ra′^{11a} or Ra′^{11b} being bonded to each other, monocycloalkene, a ring in which some carbon atoms of monocycloalkene are substituted with heteroatoms (such as an oxygen atom and a sulfur atom), or monocycloalkadiene is preferable, cycloalkene having 3 to 6 carbon atoms is preferable, and cyclopentene or cyclohexene is preferable.

The ring formed by Ra′¹⁰ and Ra′^{1a} or Ra′^{11b} being bonded to each other may be a condensed ring. Specific examples of the condensed ring include indane.

The ring formed by Ra′¹⁰ and Ra′^{1a} or Ra′^{11b} being bonded to each other may have a substituent. Examples of this substituent include Ra^{x5} described above.

Ra′^{11a} or Ra′^{11b} and Ra′¹² may be bonded to each other to form a ring, and examples of the ring include the rings formed by Ra′¹⁰ and Ra′^{11a} or Ra′^{11b} being bonded to each other.

Specific examples of the group represented by Formula (a1-r-4) are shown below.

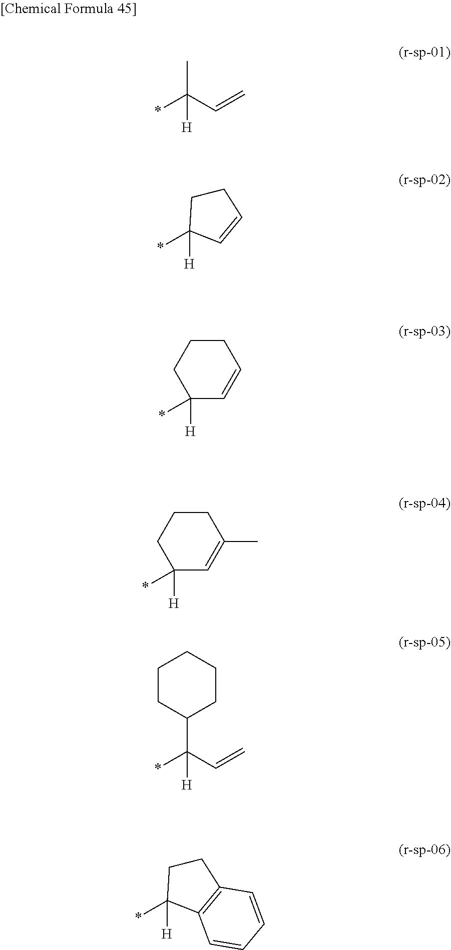

Examples of the constitutional unit (a1) include a constitutional unit derived from acrylic acid ester in which the hydrogen atom bonded to the carbon atom at the α-position may be substituted with a substituent; a constitutional unit derived from acrylamide; a constitutional unit in which at least some hydrogen atoms in a hydroxyl group of a constitutional unit derived from hydroxystyrene or a hydroxystyrene derivative are protected by a substituent containing the acid decomposable group; and a constitutional unit in which at least some hydrogen atoms in —C(═O)—OH of a constitutional unit derived from vinylbenzoic acid or a vinylbenzoic acid derivative are protected by a substituent containing the acid decomposable group.

Among the examples, as the constitutional unit (a1), a constitutional unit derived from acrylic acid ester in which the hydrogen atom bonded to the carbon atom at the α-position may be substituted with a substituent is preferable. Specific preferred examples of such a constitutional unit (a1) include a constitutional unit represented by General Formula (a1-1), General Formula (a1-2), or General Formula (a1-3).

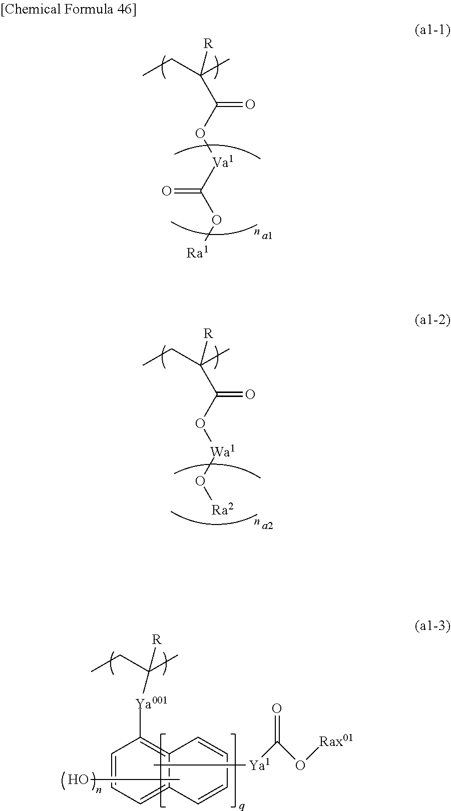

[In the formula, R represents a hydrogen atom, an alkyl group having 1 to 5 carbon atoms, or a halogenated alkyl group having 1 to 5 carbon atoms. Va¹ represents a divalent hydrocarbon group which may contain an ether bond. nₐ₁ represents an integer of 0 to 2. Ra¹ represents an acid dissociable group represented by General Formula (a1-r-1), (a1-r-2), or (a1-r-4). Wa¹ represents an (nₐ₂+1)-valent hydrocarbon group. nₐ₂ represents an integer of 1 to 3. Ra² represents an acid dissociable group represented by Formula (a1-r-1) or (a1-r-3). Ya⁰⁰¹ represents a single bond or a divalent linking group. Ya¹ represents a single bond or a divalent linking group. Rax⁰¹ represents an acid dissociable group represented by General Formula (a1-r-1), (a1-r-2), or (a1-r-4). q represents an integer of 0 to 3. n represents an integer of 1 or greater. Here, n≤q×2+4 is satisfied.]

In Formulae (a1-1) to (a1-3), as the alkyl group having 1 to 5 carbon atoms as R, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

R represents preferably a hydrogen atom, an alkyl group having 1 to 5 carbon atoms, or a fluorinated alkyl group having 1 to 5 carbon atoms and most preferably a hydrogen atom or a methyl group from the viewpoint of the industrial availability.

In Formula (a1-1), the divalent hydrocarbon group as Va¹ may be an aliphatic hydrocarbon group or an aromatic hydrocarbon group.

The aliphatic hydrocarbon group as the divalent hydrocarbon group represented by Va¹ may be saturated or unsaturated. In general, it is preferable that the aliphatic hydrocarbon group is saturated.

More specific examples of the aliphatic hydrocarbon group include a linear or branched aliphatic hydrocarbon group and an aliphatic hydrocarbon group having a ring in the structure thereof.

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂-], a trimethylene group [—(CH₂)₃-], a tetramethylene group [—(CH₂)₄-], and a pentamethylene group [—(CH₂)₅-].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Examples of the aliphatic hydrocarbon group having a ring in the structure thereof include an alicyclic hydrocarbon group (a group in which two hydrogen atoms have been removed from an aliphatic hydrocarbon ring), a group in which the alicyclic hydrocarbon group is bonded to the terminal of the linear or branched aliphatic hydrocarbon group, and a group in which the alicyclic hydrocarbon group is interposed in the middle of the linear or branched aliphatic hydrocarbon group. Examples of the linear or branched aliphatic hydrocarbon group include the same groups as those for the linear aliphatic hydrocarbon group or the branched aliphatic hydrocarbon group.

The alicyclic hydrocarbon group has preferably 3 to 20 carbon atoms and more preferably 3 to 12 carbon atoms.

The alicyclic hydrocarbon group may be monocyclic or polycyclic. The monocyclic alicyclic hydrocarbon group is preferably a group obtained by removing two hydrogen atoms from a monocycloalkane. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples thereof include adamantane, norbornane, isobornane, tricyclodecane, and tetracyclododecane.

The aromatic hydrocarbon group as the divalent hydrocarbon group represented by Va¹ is a hydrocarbon group having an aromatic ring.

The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 12 carbon atoms. Here, the number of carbon atoms in a substituent is not included in the number of carbon atoms.

Specific examples of the aromatic ring contained in the aromatic hydrocarbon group include aromatic hydrocarbon rings such as benzene, biphenyl, fluorene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom.

Specific examples of the aromatic hydrocarbon group include a group in which two hydrogen atoms have been removed from the above-described aromatic hydrocarbon ring (an arylene group); and a group in which one hydrogen atom of a group (an aryl group) formed by removing one hydrogen atom from the aromatic hydrocarbon ring has been substituted with an alkylene group (for example, a group formed by further removing one more hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (a1-1), Ra¹ represents preferably an acid dissociable group represented by General Formula (a1-r-2) or (a1-r-4) and, among these, more preferably a group represented by General Formula (a1-r2-1) or an acid dissociable group represented by General Formula (a1-r-4).

In Formula (a1-2), the (nₐ₂+1)-valent hydrocarbon group as Wa¹ may be an aliphatic hydrocarbon group or an aromatic hydrocarbon group. The aliphatic hydrocarbon group indicates a hydrocarbon group that has no aromaticity and may be saturated or unsaturated. In general, it is preferable that the aliphatic hydrocarbon group is saturated. Examples of the aliphatic hydrocarbon group include a linear or branched aliphatic hydrocarbon group, an aliphatic hydrocarbon group having a ring in the structure thereof, and a group obtained by combining the linear or branched aliphatic hydrocarbon group and the aliphatic hydrocarbon group having a ring in the structure thereof. The valency of nₐ₂+1 is preferably divalent, trivalent, or tetravalent and more preferably divalent or trivalent.

In Formula (a1-2), it is preferable that Ra² represents an acid dissociable group represented by General Formula (a1-r-1).

In Formula (a1-3), the divalent linking group as Ya⁰⁰¹ is not particularly limited, and suitable examples thereof include a divalent hydrocarbon group which may have a substituent and a divalent linking group having a heteroatom.

Among these, it is preferable that Ya⁰⁰¹ represents preferably an ester bond [—C(═O)—O— or —O—C(═O)—], an ether bond (—O—), a linear or branched alkylene group, an aromatic hydrocarbon group, a combination thereof, or a single bond. The alkylene group has preferably 1 to 10 carbon atoms, more preferably 1 to 6 carbon atoms, still more preferably 1 to 4 carbon atoms, and particularly preferably 1 to 3 carbon atoms. Among these, Ya⁰⁰¹ represents more preferably a combination of an ester bond [—C(═O)—O— or —O—C(═O)—] and a linear alkylene group, or a single bond and still more preferably a single bond.

In Formula (a1-3), the divalent linking group as Ya¹ is not particularly limited, and suitable examples thereof include a divalent hydrocarbon group which may have a substituent and a divalent linking group having a heteroatom.

Among these, it is preferable that Ya¹ represents an ester bond [—C(═O)—O— or —O—C(═O)—], an ether bond (—O—), a linear or branched alkylene group, an aromatic hydrocarbon group, a combination thereof, or a single bond. Among these, Ya¹ represents more preferably a combination of an ester bond [—C(═O)—O— or —O—C(═O)—]and a linear alkylene group, or a single bond and still more preferably a single bond.

In Formula (a1-3), Rax⁰¹ represents preferably an acid dissociable group represented by General Formula (a1-r-2) or (a1-r-4) and, among these, more preferably an acid dissociable group represented by General Formula (a1-r-2) and still more preferably a group represented by General Formula (a1-r2-1).

In General Formula (a1-3), q represents an integer of 0 to 3. A benzene structure is formed in a case where q represents 0, a naphthalene structure is formed in a case where q represents 1, an anthracene structure is formed in a case where q represents 2, and a tetracene structure is formed in a case where q represents 3.

In General Formula (a1-3), n represents an integer of 1 or greater, preferably an integer of 1 to 5, more preferably an integer of 1 to 3, and still more preferably an integer of 1 or 2.

In Formula (a1-3), n≤q×2+4 is satisfied. For example, in a case where q represents 1 and thus a naphthalene structure is formed, all six hydrogen atoms of the naphthalene may be substituted with hydroxy groups. In addition, the substitution positions of Ya⁰⁰¹, the -Ya¹-C(═O)—O—Rax⁰¹ group, and the hydroxy group in the naphthalene are not particularly limited.

Specific examples of the constitutional unit (a1) are shown below. In the formulae shown below, R^{α} represents a hydrogen atom, a methyl group, or a trifluoromethyl group.

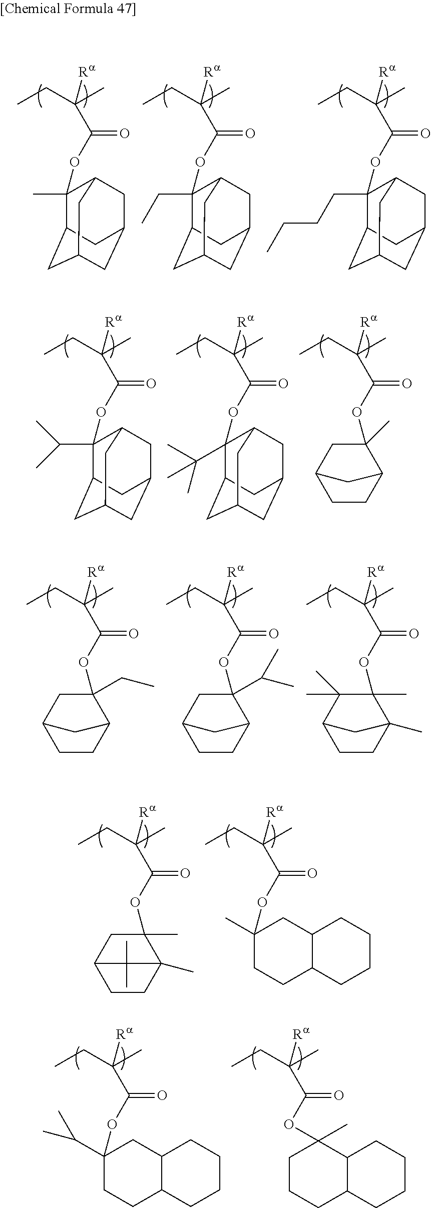

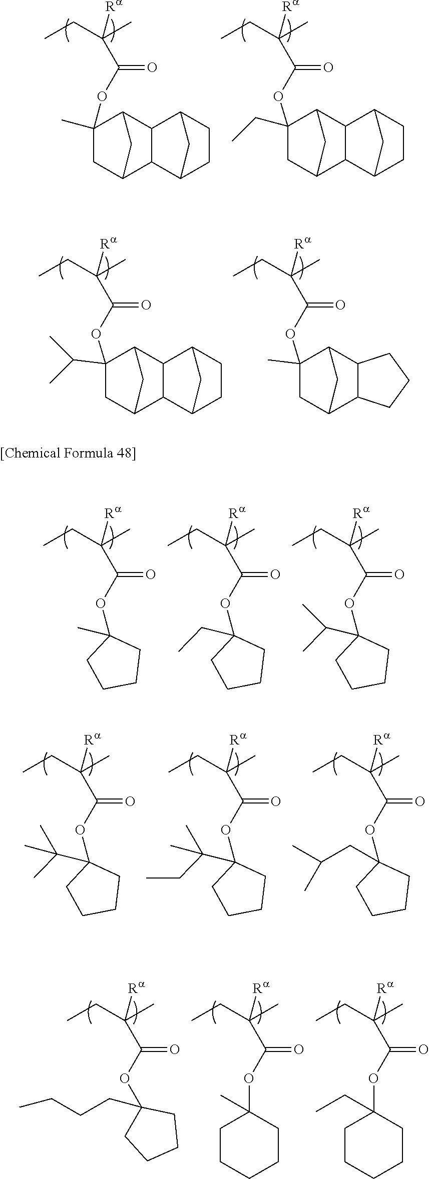

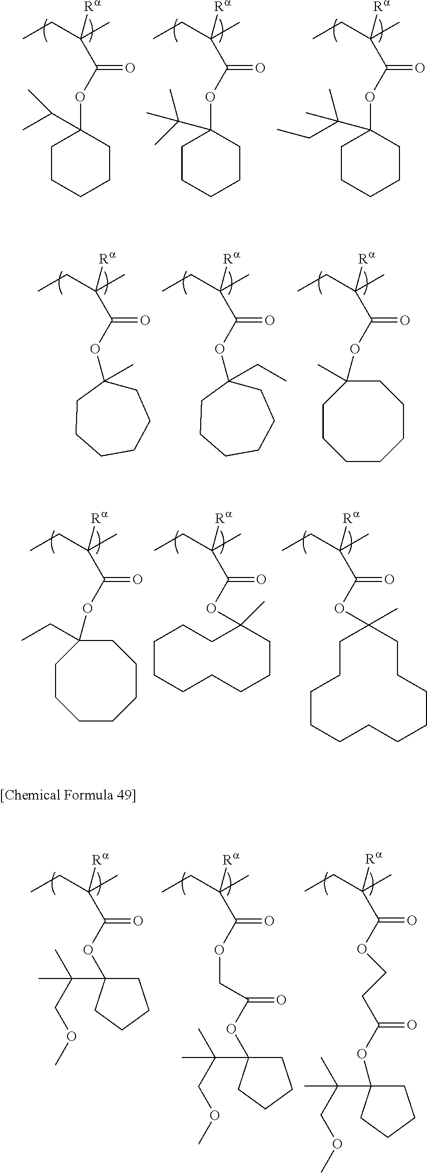

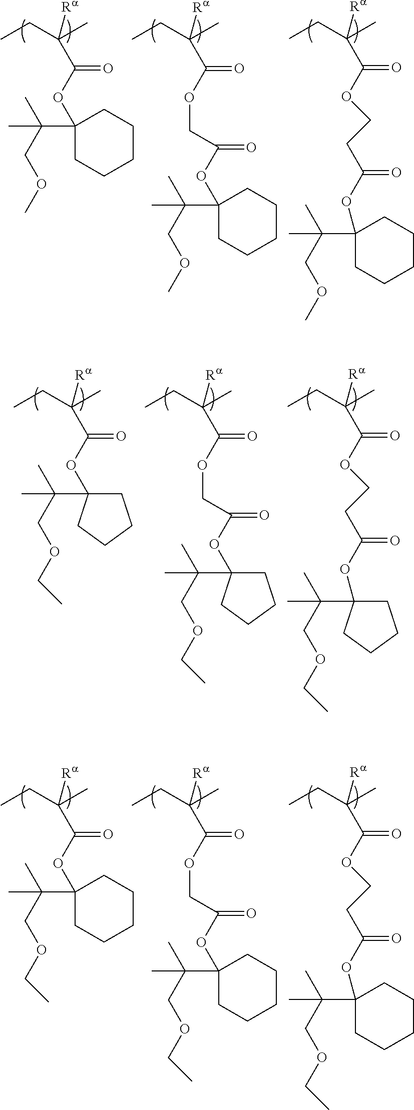

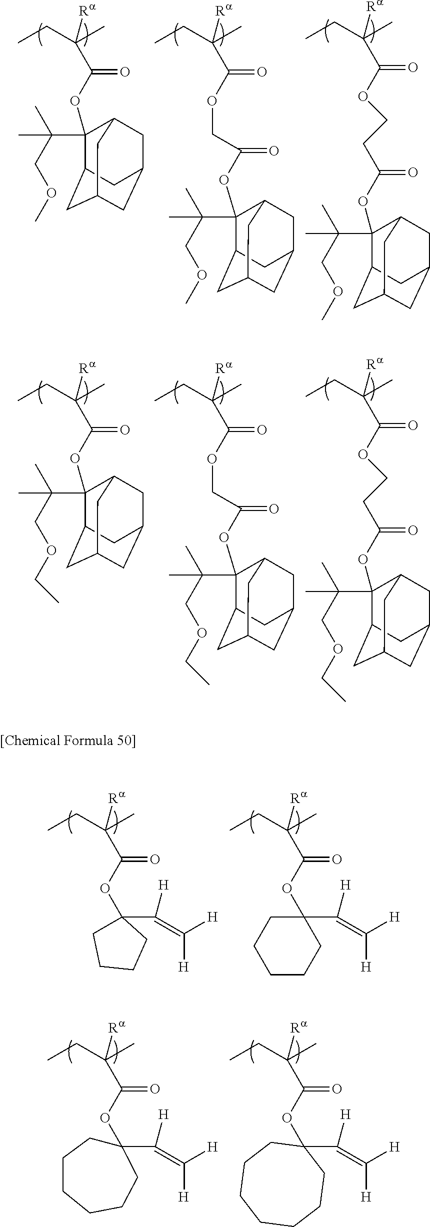

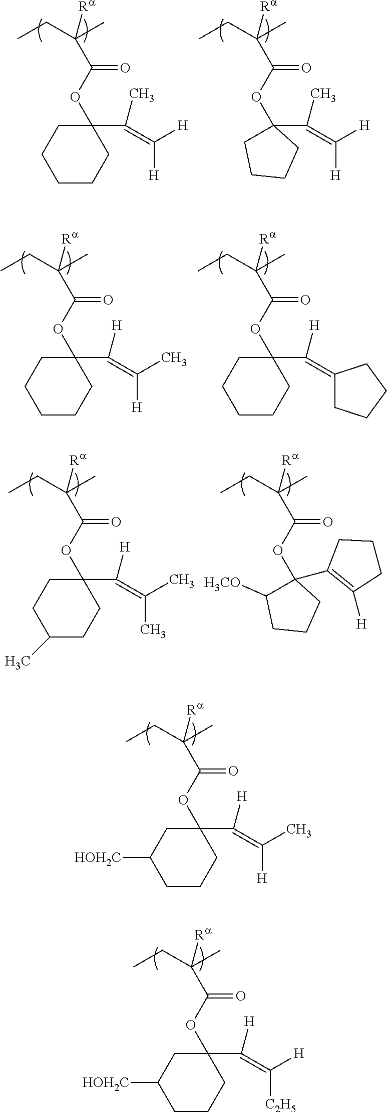

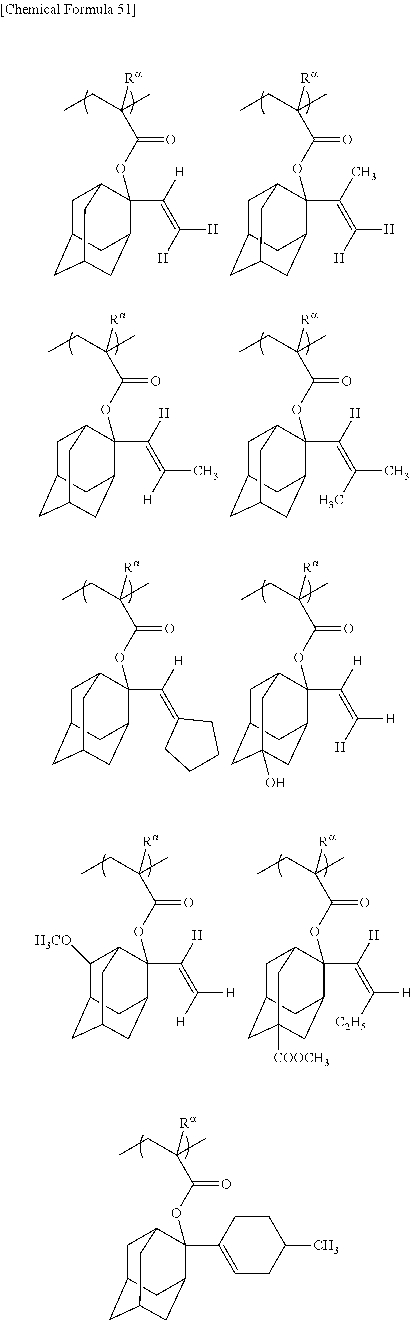

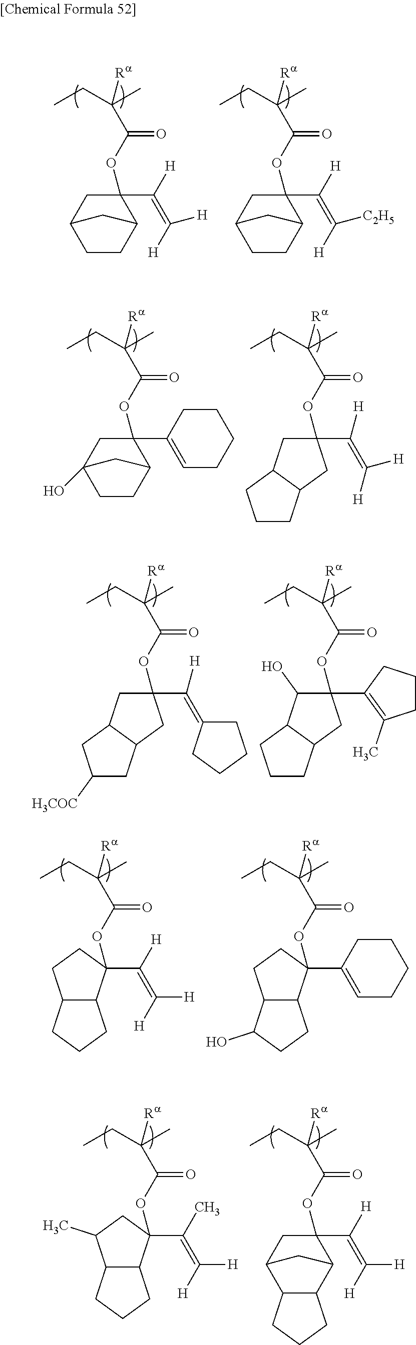

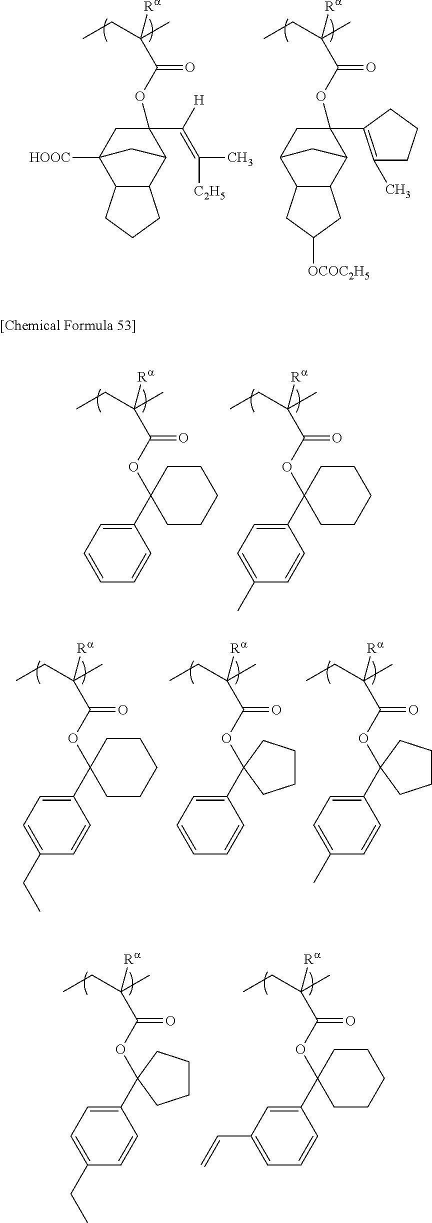

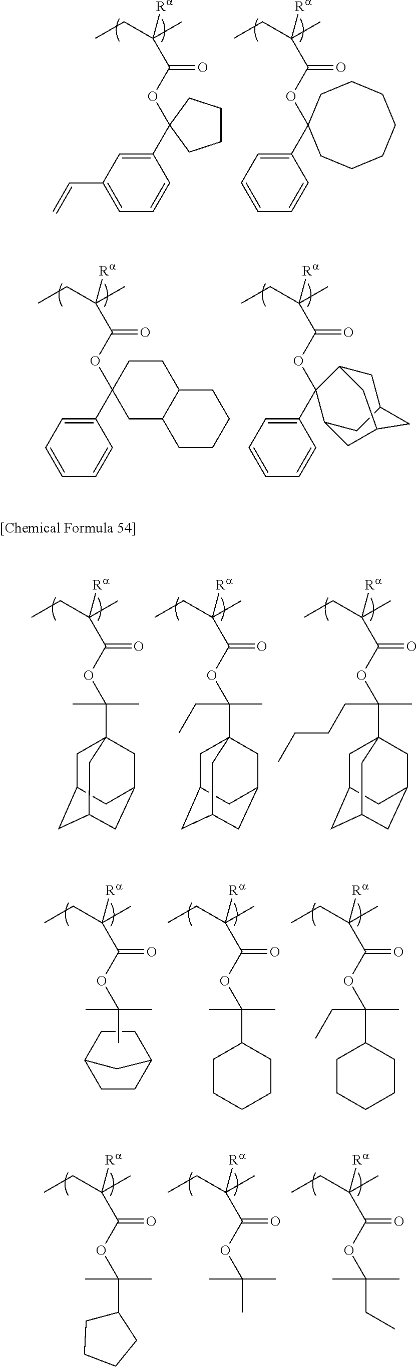

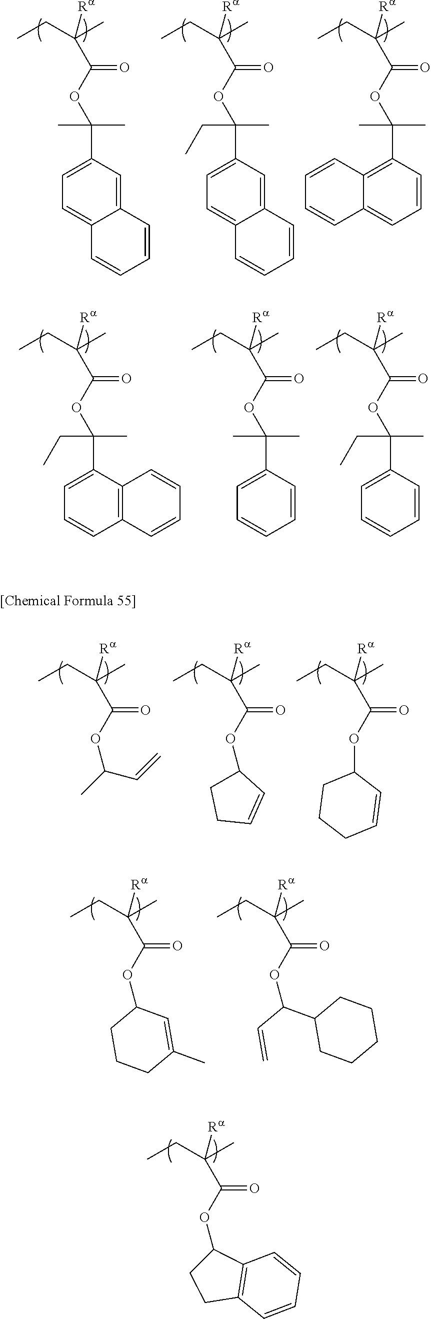

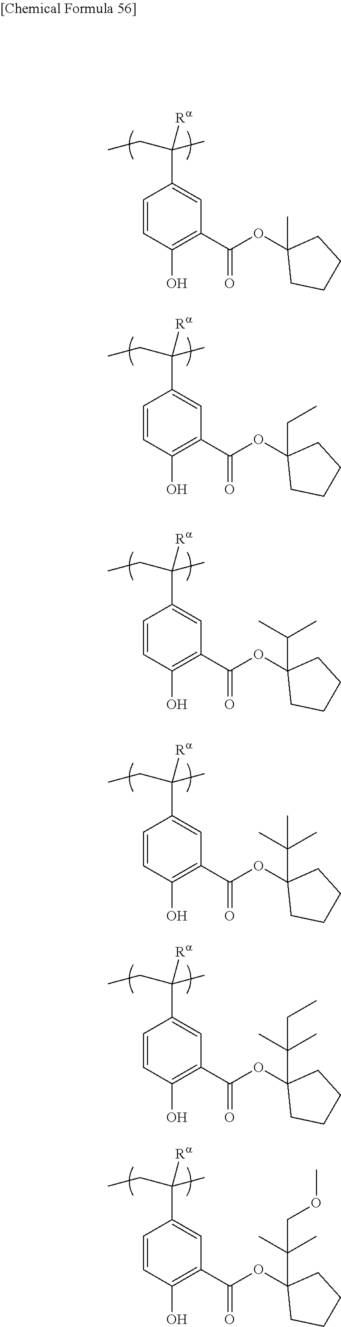

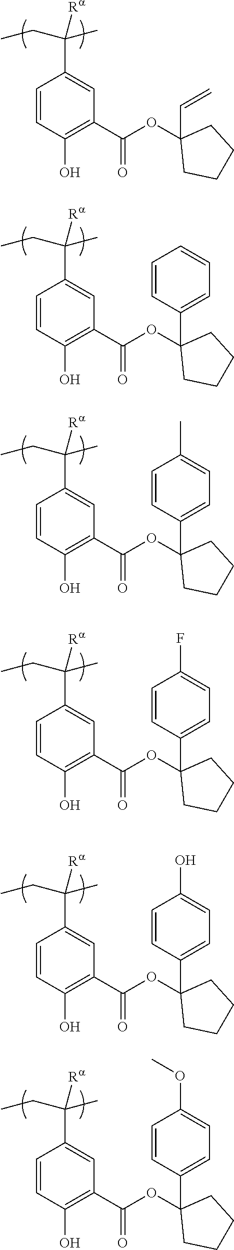

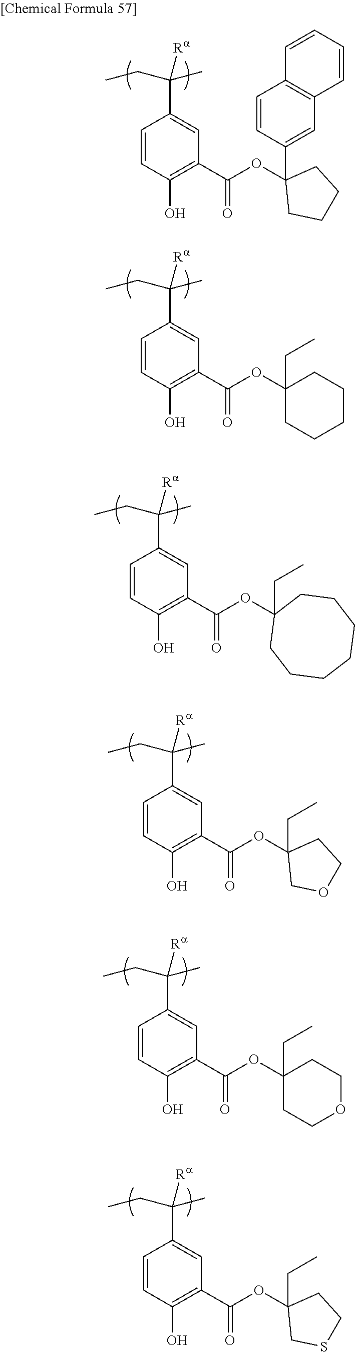

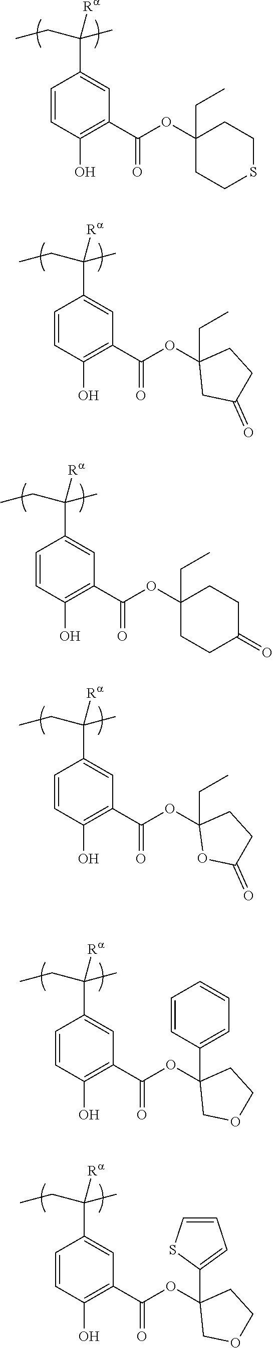

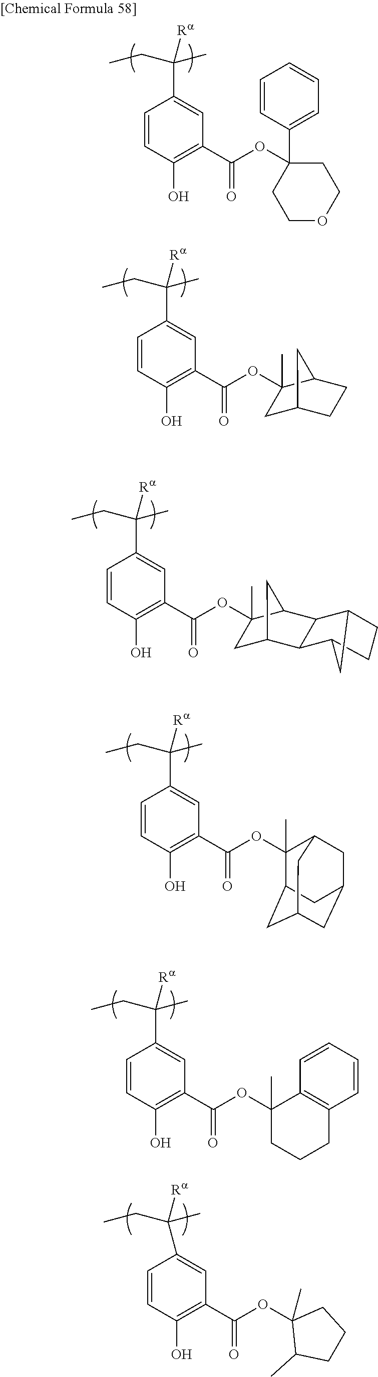

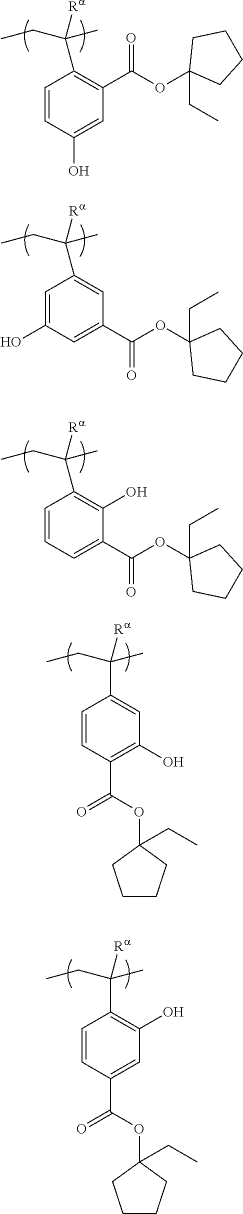

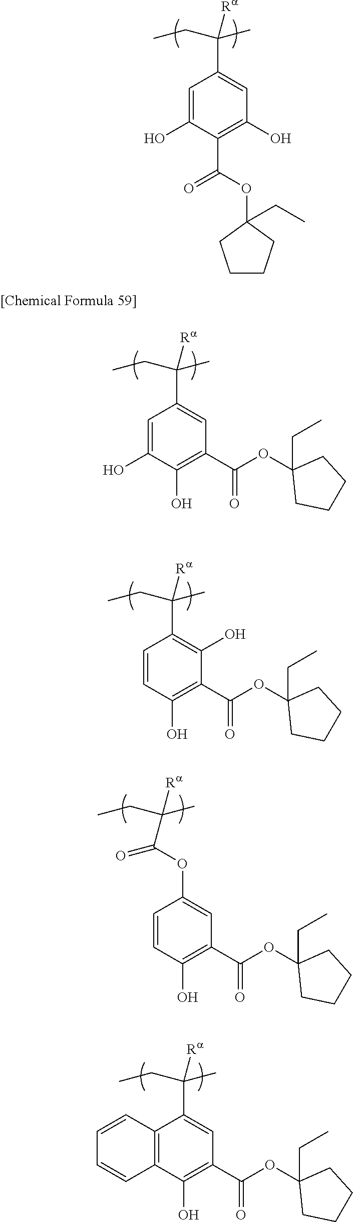

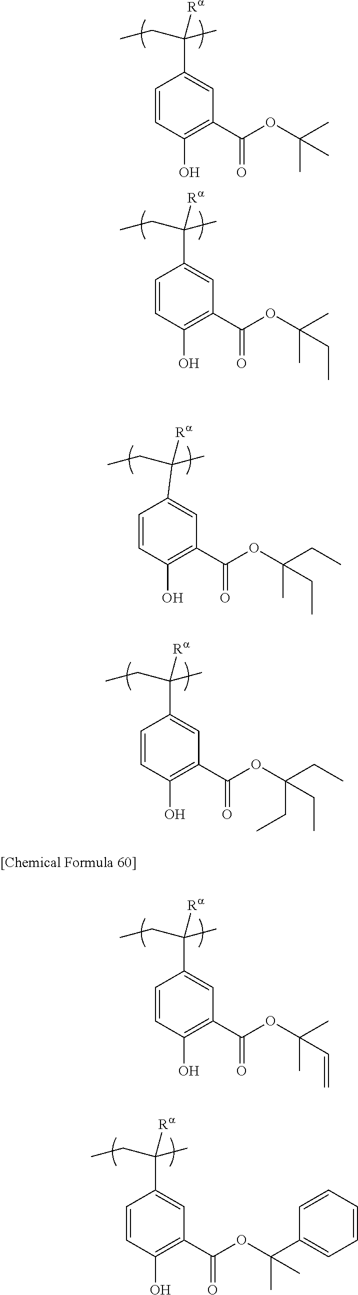

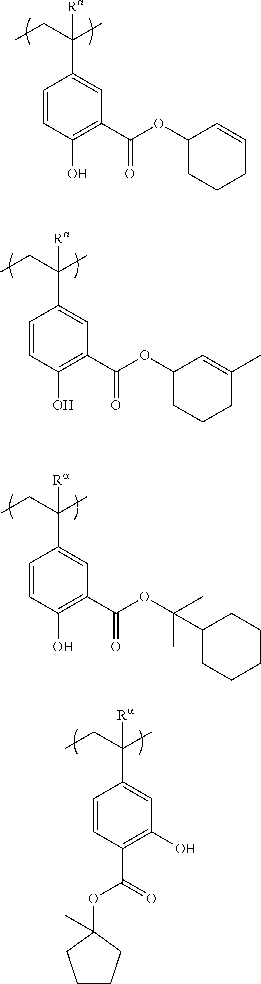

The constitutional unit (a1) included in the component (A1) may be used alone or two or more kinds thereof.

From the viewpoint of further easily enhancing the lithography characteristics (the sensitivity, the shape, and the like) using electron beams or EUV, a constitutional unit represented by Formula (a1-1) or a constitutional unit represented by Formula (a1-3) is more preferable as the constitutional unit (a1).

Among these, from the viewpoint of suitably increasing the reactivity for EB or EUV, the acid dissociable groups (Ra¹, Rax⁰¹) are each preferably an acid dissociable group represented by General Formula (a1-r2-1), (a1-r2-3), (a1-r2-4), or (a1-r-4) and, among these, particularly preferably a cyclic group.

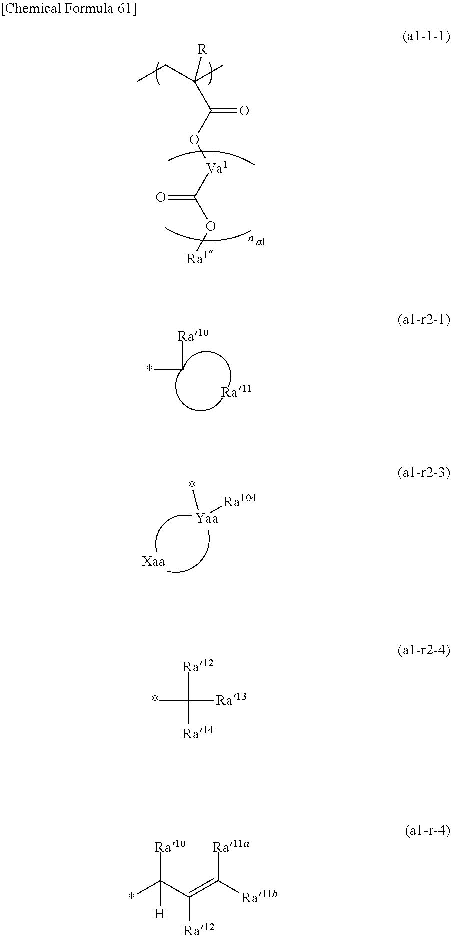

[In the formulae, Ra¹″ represents an acid dissociable group represented by General Formula (a1-r2-1), (a1-r2-3), (a1-r2-4), or (a1-r-4)]* represents a bonding site.]

In Formula (a1-1-1), R, Va¹, and nₐ₁ each have the same definition as that for R, Va¹, and nₐ₁ in Formula (a1-1).

The acid dissociable group represented by General Formula (a1-r2-1), (a1-r2-3), (a1-r2-4), or (a1-r-4) is as described above. Among these, it is preferable to select those in which the acid dissociable group is a cyclic group because the reactivity is enhanced for EB or EUV, which is preferable.

The proportion of the constitutional unit (a1) in the component (A1) is preferably in a range of 20% to 75% by mole, more preferably in a range of 25% to 70% by mole, still more preferably in a range of 30% to 60% by mole, and particularly preferably in a range of 35% to 55% by mole with respect to the total amount (100% by mole) of all constitutional units constituting the component (A1).

In a case where the proportion of the constitutional unit (a1) is set to be greater than or equal to the lower limits of the above-described preferable ranges, high sensitivity and etching resistance are likely to be achieved, and the roughness and the degree of light exposure margin are likely to be improved. Further, in a case where the proportion of the constitutional unit (a1) is less than or equal to the upper limits of the above-described preferable ranges, the constitutional unit (a1) and other constitutional units can be balanced, and the lithography characteristics are improved.

In regard to constitutional unit (a10):

The constitutional unit (a10) is a constitutional unit represented by General Formula (a10-1).

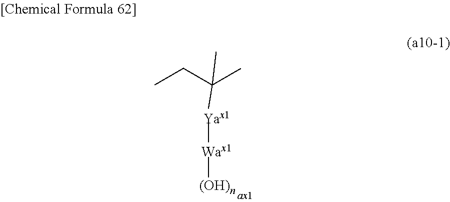

[In Formula (a10-1), R represents an alkyl group having 1 to 5 carbon atoms, a halogenated alkyl group having 1 to 5 carbon atoms, or a hydrogen atom. Ya^{x1} represents a divalent linking group or a single bond. Wa^{x1} represents an aromatic hydrocarbon group which may have a substituent. nₐₓ₁ represents an integer of 1 or greater as long as the valence allows it.

In Formula (a10-1), R has the same definition as that for R in General Formula (a1-1).

R represents preferably a hydrogen atom, an alkyl group having 1 to 5 carbon atoms, or a fluorinated alkyl group having 1 to 5 carbon atoms, and from the viewpoint of industrial availability, more preferably a hydrogen atom, a methyl group, or trifluoromethyl group, still more preferably a hydrogen atom or a methyl group, and particularly preferably a hydrogen atom.

In Formula (a10-1), Ya^{x1} represents a single bond or a divalent linking group.

In the chemical formula, the divalent linking group as Ya^{x1} is not particularly limited, and suitable examples thereof include a divalent hydrocarbon group which may have a substituent and a divalent linking group having a heteroatom.

Divalent Hydrocarbon Group which May have Substituent:

The divalent hydrocarbon group which may have a substituent may be an aliphatic hydrocarbon group or an aromatic hydrocarbon group.

### Aliphatic Hydrocarbon Group

The aliphatic hydrocarbon group indicates a hydrocarbon group that has no aromaticity. The aliphatic hydrocarbon group may be saturated or unsaturated. In general, it is preferable that the aliphatic hydrocarbon group is saturated. Examples of the aliphatic hydrocarbon group include a linear or branched aliphatic hydrocarbon group and an aliphatic hydrocarbon group having a ring in the structure thereof.

### Linear or Branched Aliphatic Hydrocarbon Group

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable. Specific examples thereof include a methylene group [—CH₂—], an ethylene group [—(CH₂)₂-], a trimethylene group [—(CH₂)₃-], a tetramethylene group [—(CH₂)₄-], and a pentamethylene group [—(CH₂)₅-].

The branched aliphatic hydrocarbon group preferably has 2 to 10 carbon atoms, more preferably has 3 to 6 carbon atoms, still more preferably has 3 or 4 carbon atoms, and most preferably has 3 carbon atoms. As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

The linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

### Aliphatic Hydrocarbon Group Having Ring in Structure Thereof

Examples of the aliphatic hydrocarbon group having a ring in the structure thereof include a cyclic aliphatic hydrocarbon group which may have a substituent having a heteroatom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. As the linear or branched aliphatic hydrocarbon group, the same groups as those described above are exemplary examples.

The cyclic aliphatic hydrocarbon group preferably has 3 to 20 carbon atoms and more preferably has 3 to 12 carbon atoms.

The cyclic aliphatic hydrocarbon group may be a polycyclic group or a monocyclic group. The monocyclic alicyclic hydrocarbon group is preferably a group obtained by removing two hydrogen atoms from a monocycloalkane. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples of the polycycloalkane include adamantane, norbornane, isobornane, tricyclodecane, and tetracyclododecane.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is still more preferable. As the halogen atom as the substituent, a fluorine atom is preferable.

Examples of the halogenated alkyl group as the substituent include groups in which some or all hydrogen atoms in the above-described alkyl groups are substituted with the above-described halogen atoms.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂—O— is preferable.

### Aromatic Hydrocarbon Group

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the number of carbon atoms.

Specifically, as the aromatic ring, an aromatic hydrocarbon ring such as benzene, naphthalene, anthracene, and phenanthrene; and an aromatic heterocyclic ring in which some carbon atoms constituting the aromatic hydrocarbon ring have been substituted with heteroatoms are exemplary examples. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group in which two hydrogen atoms have been removed from the above-described aromatic hydrocarbon ring or aromatic heterocyclic ring (an arylene group or a heteroarylene group); a group in which two hydrogen atoms have been removed from an aromatic compound having two or more aromatic rings (such as biphenyl or fluorene); and a group in which one hydrogen atom of a group (an aryl group or a heteroaryl group) obtained by removing one hydrogen atom from the above-described aromatic hydrocarbon ring or aromatic heterocyclic ring has been substituted with an alkylene group (for example, a group obtained by further removing one hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aryl group or the heteroaryl group is preferably in a range of 1 to 4, more preferably 1 or 2, and particularly preferably 1.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group, the halogen atom, and the halogenated alkyl group as the substituents, the groups described as the substituents that substitute a hydrogen atom in the cyclic aliphatic hydrocarbon group are exemplary examples.

### Divalent Linking Group Having Heteroatom:

As the divalent linking group containing a heteroatom, —O—, —C(═O)—O—, —O—C(═O)—, —C(═O)—O—, —O—C(═O)—O—, —C(═O)—NH—, —NH—, —NH—C(═NH)—(H may be substituted with a substituent such as an alkyl group and an acyl group), —S—, —S(═O)₂—, —S(═O)₂—O—, and a group represented by General Formulae —Y²¹-Y²²—, —Y²¹—O—, —Y²¹—C(═O)—O—, —C(═O)—O—Y²¹—, —Y²¹—C(═O)—O]ₘ, —Y²²—, —Y²¹—O—C(═O)—Y²²—, or —Y²¹—S(═O)₂—O—Y²²—[in the formulae, Y²¹ and Y²² each independently represent a divalent hydrocarbon group which may have a substituent, O represents an oxygen atom, and m″ represents an integer of 0 to 3] are exemplary examples.

In a case where the above-described divalent linking group containing a heteroatom is —C(═O)—NH—, —C(═O)—NH—C(═O)—, —NH—, or —NH—C(═NH)—, H may be substituted with a substituent such as an alkyl group and an acyl group. The substituent (alkyl group, acyl group, and the like) preferably has 1 to 10 carbon atoms, more preferably has 1 to 8 carbon atoms, and particularly preferably has 1 to 5 carbon atoms.

In General Formula —Y²¹-Y²²—, —Y²¹—, —Y²¹—C(═O)—O—, —C(═O)—O—Y²¹—, —[Y²¹—C(═O)—O]ₘ, —Y²²—, —Y²¹—O—C(═O)—Y²²—, or —Y²¹—S(═O)₂—O—Y²²—, Y²¹ and Y²² each independently represent a divalent hydrocarbon group which may have a substituent. As the divalent hydrocarbon group, the same ones as those described above are exemplary examples.

As Y²¹, a linear aliphatic hydrocarbon group is preferable, a linear alkylene group is more preferable, a linear alkylene group having 1 to 5 carbon atoms is still more preferable, and a methylene group or an ethylene group is particularly preferable.

As Y²², a linear or branched aliphatic hydrocarbon group is preferable, and a methylene group, an ethylene group, or an alkylmethylene group is more preferable. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y²¹—C(═O)—O]ₘ″-Y²²—, m″ represents an integer of 0 to 3, preferably an integer of 0 to 2, more preferably 0 or 1, and particularly preferably 1. That is, a group represented by Formula —Y²¹—C(═O)—O—Y²²— is preferable as the group represented by Formula —[Y²¹—C(═O)—O]ₘ″-Y²²—. Among these, a group represented by Formula —(CH₂)ₐ′-C(═O)—O—(CH₂)_{b′}— is preferable. In the formula, a′ represents an integer of 1 to 10, preferably an integer of 1 to 8, more preferably an integer of 1 to 5, still more preferably 1 or 2, and most preferably 1. b′ represents an integer of 1 to 10, preferably an integer of 1 to 8, more preferably an integer of 1 to 5, still more preferably 1 or 2, and most preferably 1.

Ya^{x1} represents preferably a single bond, an ester bond [—C(═O)—O— or —O—C(═O)— an ether bond (—O—), a linear or branched alkylene group, or a combination thereof and more preferably a single bond or an ester bond [—C(═O)—O— or —O—C(═O)—].

In General Formula (a10-1), Wa^{x1} represents an aromatic hydrocarbon group which may have a substituent.

Examples of the aromatic hydrocarbon group as Wa^{x1} include a group obtained by removing (nₐₓ₁+1) hydrogen atoms from an aromatic ring which may have a substituent. The aromatic ring is not particularly limited as long as the aromatic ring is a cyclic conjugated system having (4n+2) π electrons. The aromatic ring has preferably 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a heteroatom. Examples of the heteroatom in the aromatic heterocyclic rings include an oxygen atom, a sulfur atom, and a nitrogen atom. Specific examples of the aromatic heterocyclic ring include a pyridine ring and a thiophene ring. Examples of the aromatic hydrocarbon group as Wa^{x1} also include a group obtained by removing (nₐₓ₁+1) hydrogen atoms from an aromatic compound including an aromatic ring (for example, biphenyl or fluorene) which may have two or more substituents.

Among these, Wa^{x1} represents preferably a group in which (nₐₓ₁+1) hydrogen atoms have been removed from benzene, naphthalene, anthracene, or biphenyl, more preferably a group in which (nₐₓ₁+1) hydrogen atoms have been removed from benzene or naphthalene, and still more preferably a group in which (nₐₓ₁+1) hydrogen atoms have been removed from benzene.

The aromatic hydrocarbon group as Wa^{x1} may have a substituent or may not have a substituent. Examples of substituents include an alkyl group, an alkoxy group, a halogen atom, and a halogenated alkyl group. Examples of the alkyl group, the alkoxy group, the halogen atom, and the halogenated alkyl group as the substituent include the same groups as those for the substituent of the cyclic alicyclic hydrocarbon group as Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. It is preferable that the aromatic hydrocarbon group as Wa^{x1} has no substituent.

In Formula (a10-1), nₐₓ₁ represents an integer of 1 or greater, preferably an integer of 1 to 10, more preferably an integer of 1 to 5, still more preferably 1, 2, or 3, and particularly preferably 1 or 2.

Specific examples of the constitutional unit (a10) represented by Formula (a10-1) are described below.

In the formulae shown below, R^{α} represents a hydrogen atom, a methyl group, or a trifluoromethyl group.

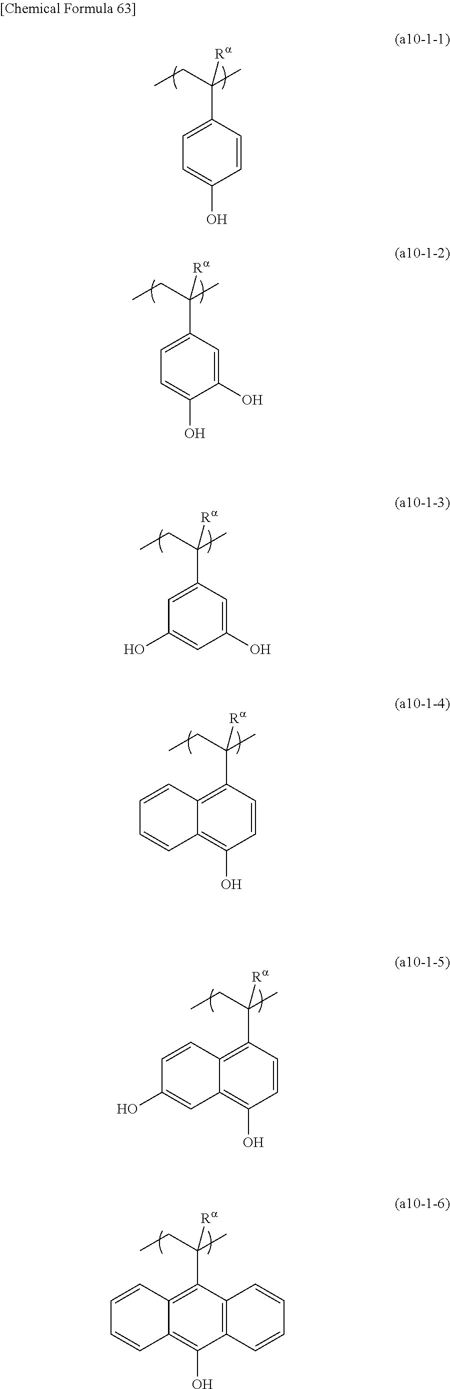

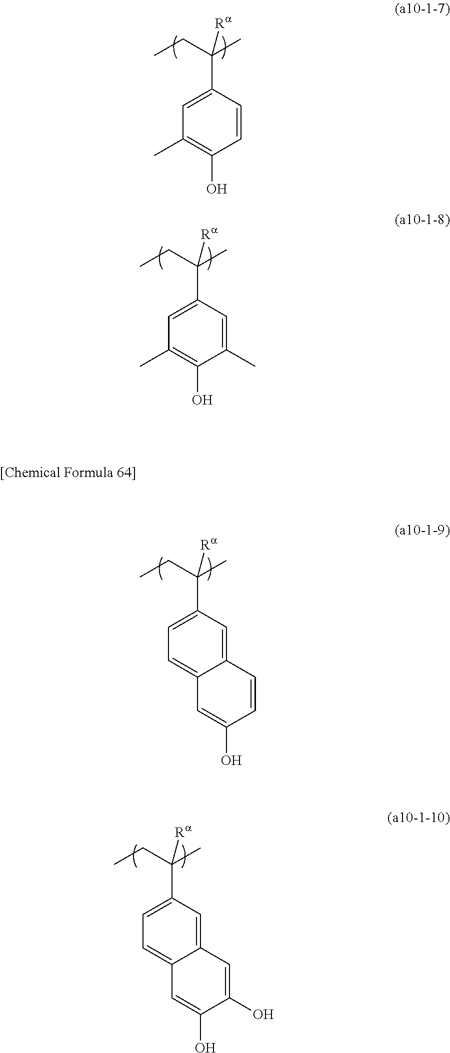

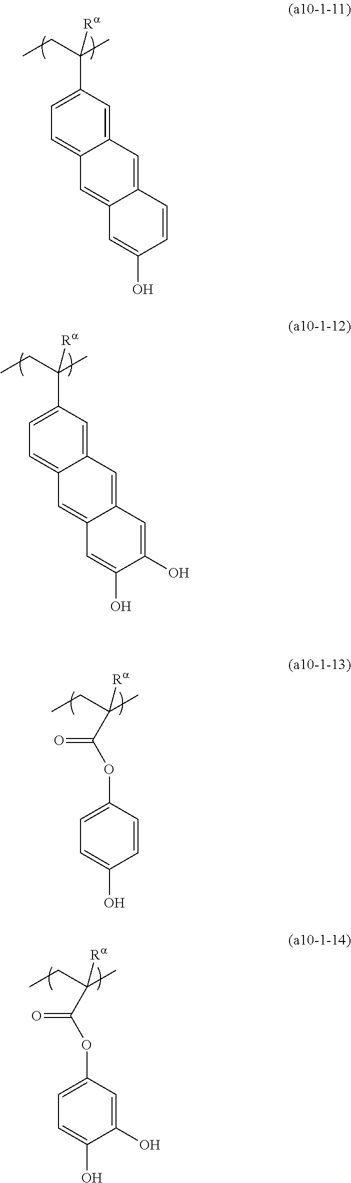

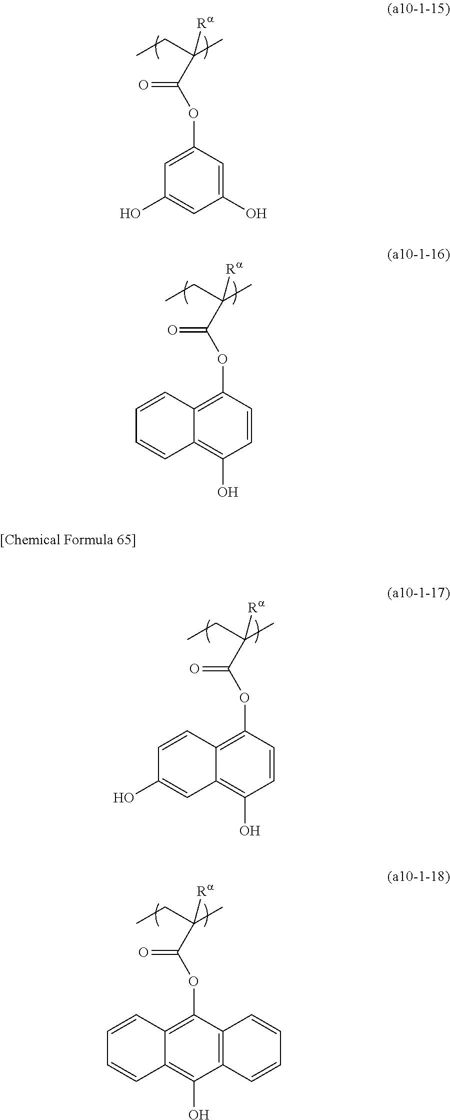

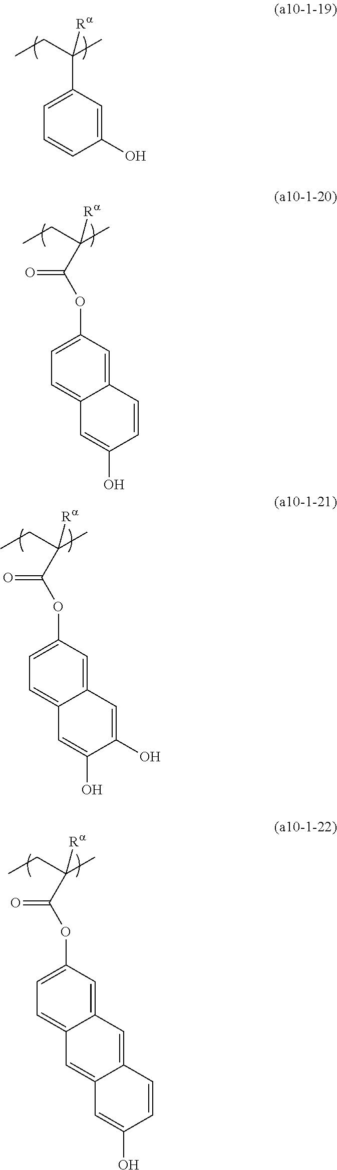

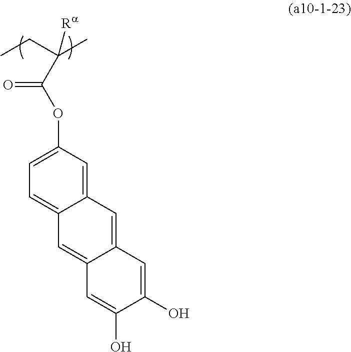

The constitutional unit (a10) that can be included in the component (A1) may be used alone or two or more kinds thereof.

The component (A1) may or may not have the constitutional unit (a10), but it is preferable that the component (A1) has the constitutional unit (a10).

In a case where the component (A1) has the constitutional unit (a10), the proportion of the constitutional unit (a10) in the component (A1) is preferably in a range of 10% to 60% by mole, more preferably in a range of 20% to 50% by mole, still more preferably in a range of 25% to 45% by mole, and particularly preferably in a range of 30% to 40% by mole with respect to the total amount (100% by mole) of all constitutional units constituting the component (A1).

In a case where the proportion of the constitutional unit (a10) is set to be greater than or equal to the above-described lower limits, the sensitivity is likely to be enhanced. Meanwhile, in a case where the proportion thereof is set to be less than or equal to the above-described upper limits, the constitutional unit (a10) and other constitutional units are likely to be balanced.

### In Regard to Constitutional Unit (a2):

The component (A1) may further have a constitutional unit (a2) (here, a constitutional unit corresponding to the constitutional unit (a1) is excluded) containing a lactone-containing cyclic group.

In a case where the component (A1) is used to form a resist film, the lactone-containing cyclic group of the constitutional unit (a2) is effective for increasing the adhesiveness of the resist film to the substrate. Further, in a case where the component (A1) contains the constitutional unit (a2), the lithography characteristics and the like are improved due to the effects of appropriately adjusting the acid diffusion length, increasing the adhesiveness of the resist film to the substrate, and appropriately adjusting the solubility during the development.

The term “lactone-containing cyclic group” indicates a cyclic group that has a ring (lactone ring) containing —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group in the constitutional unit (a2) is not particularly limited, and an optional constitutional unit can be used. Specific examples thereof include groups each represented by General Formulae (a2-r-1) to (a2-r-7).

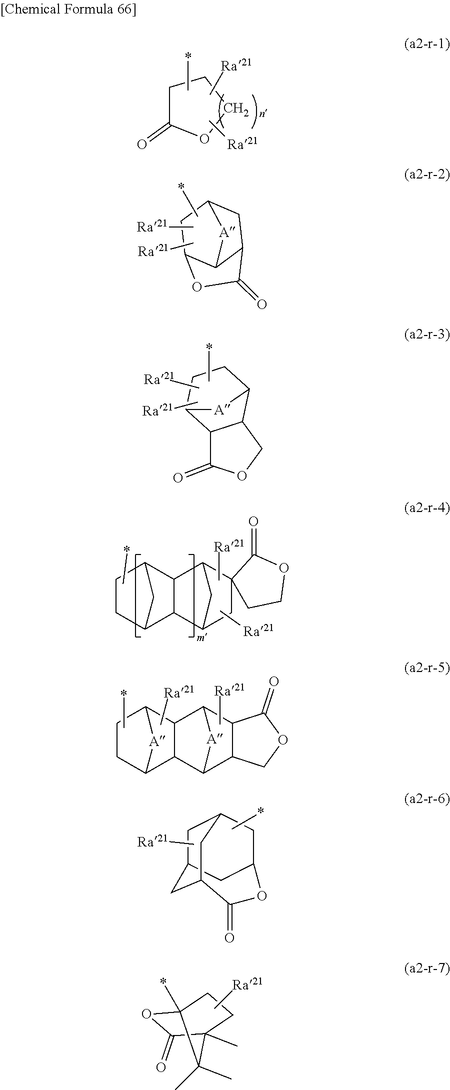

[In the formulae, Ra′²¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, or a lactone-containing cyclic group, A″ represents an alkylene group having 1 to 5 carbon atoms which may have an oxygen atom (—O—) or a sulfur atom (—S—), an oxygen atom, or a sulfur atom, n′ represents an integer of 0 to 2, and m′ represents 0 or 1. * represents a bonding site (the same applies hereinafter).]

In Formulae (a2-r-1) to (a2-r-7), it is preferable that the alkyl group as Ra′²¹ is an alkyl group having 1 to 6 carbon atom. Further, it is preferable that the alkyl group is linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ is an alkoxy group having 1 to 6 carbon atoms. Further, it is preferable that the alkoxy group is linear or branched. Specific examples of the alkoxy groups include a group formed by linking the above-described alkyl group exemplified as the alkyl group represented by Ra′²¹ to an oxygen atom (—O—).

As the halogen atom as Ra′²¹, a fluorine atom is preferable.

Examples of the halogenated alkyl group as Ra′²¹ include groups in which some or all hydrogen atoms in the alkyl group as Ra′²¹ have been substituted with the halogen atoms. As the halogenated alkyl group, a fluorinated alkyl group is preferable, and a perfluoroalkyl group is particularly preferable.

In —COOR″ and —OC(═O)R″ as Ra′²¹, each R″ represents a hydrogen atom, an alkyl group, or a lactone-containing cyclic group.

The alkyl group as R″ may be linear, branched, or cyclic and has preferably 1 to 15 carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, the number of carbon atoms thereof is preferably in a range of 3 to 15, more preferably in a range of 4 to 12, and most preferably in a range of 5 to 10. Specific examples thereof include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as bicycloalkane, tricycloalkane, or tetracycloalkane. More specific examples thereof include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane, or tetracyclododecane.

Examples of the lactone-containing cyclic group as R″ include the same groups as those for the groups each represented by General Formulae (a2-r-1) to (a2-r-7).

As the hydroxyalkyl group as Ra′²¹, a hydroxyalkyl group having 1 to 6 carbon atoms is preferable, and specific examples thereof include a group in which at least one hydrogen atom in the alkyl group as Ra′²¹ has been substituted with a hydroxyl group.

Among the examples, it is preferable that each Ra′²¹ independently represent a hydrogen atom or a cyano group.

In General Formulae (a2-r-2), (a2-r-3) and (a2-r-5), as the alkylene group having 1 to 5 carbon atoms as A″, a linear or branched alkylene group is preferable, and examples thereof include a methylene group, an ethylene group, an n-propylene group, and an isopropylene group. Specific examples of the alkylene groups that contain an oxygen atom or a sulfur atom include a group obtained by interposing —O— or —S— in the terminal of the alkylene group or between the carbon atoms of the alkylene group, and examples thereof include —O—CH₂—, —CH₂—O—CH₂—, —S—CH₂—, and —CH₂—S—CH₂—. A″ represents preferably an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below.

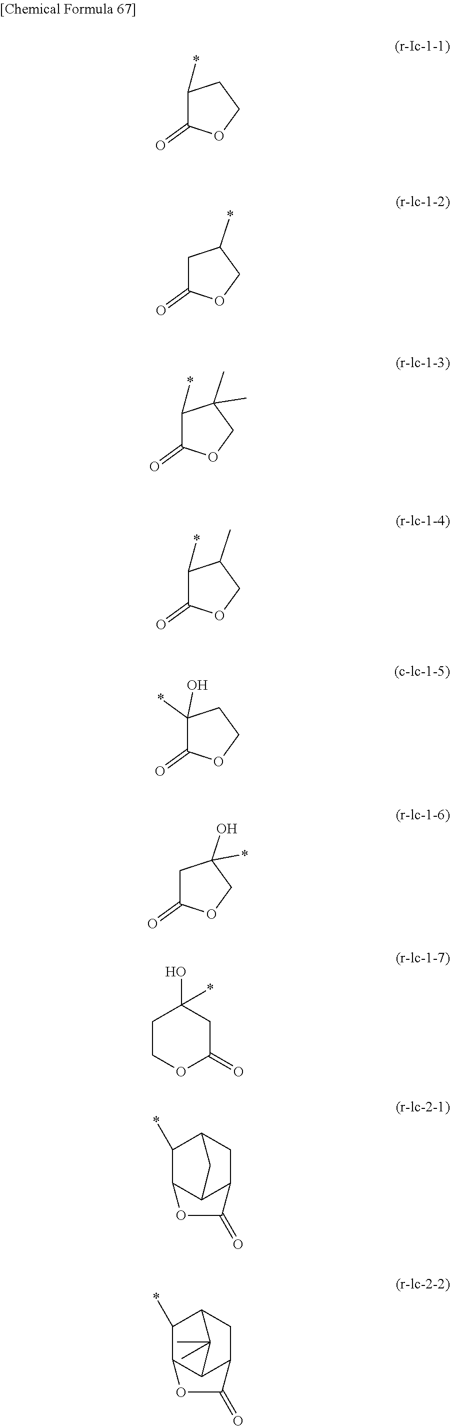

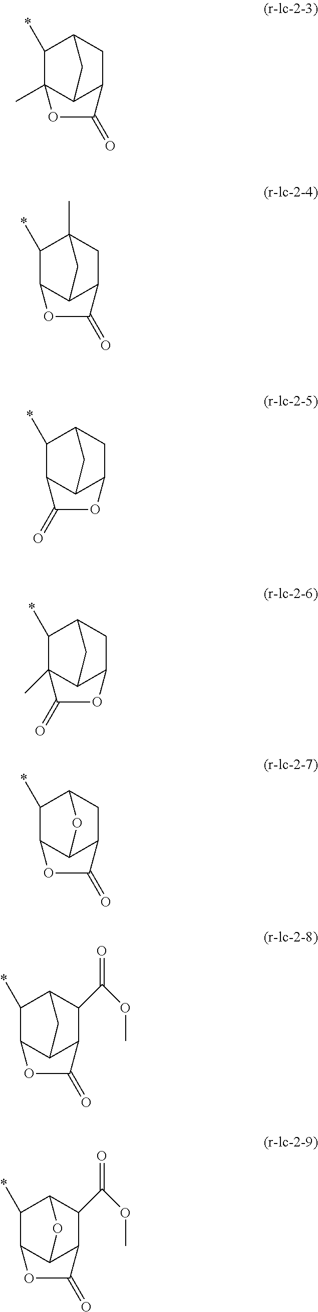

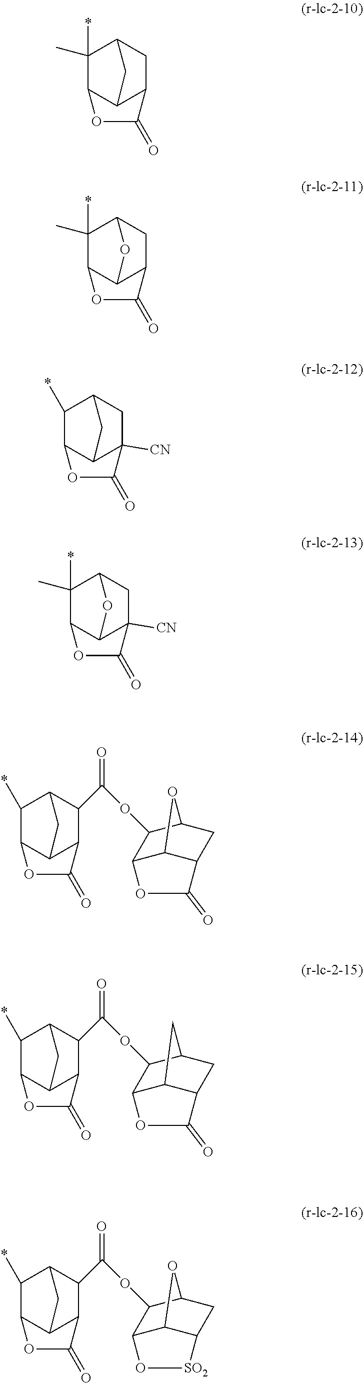

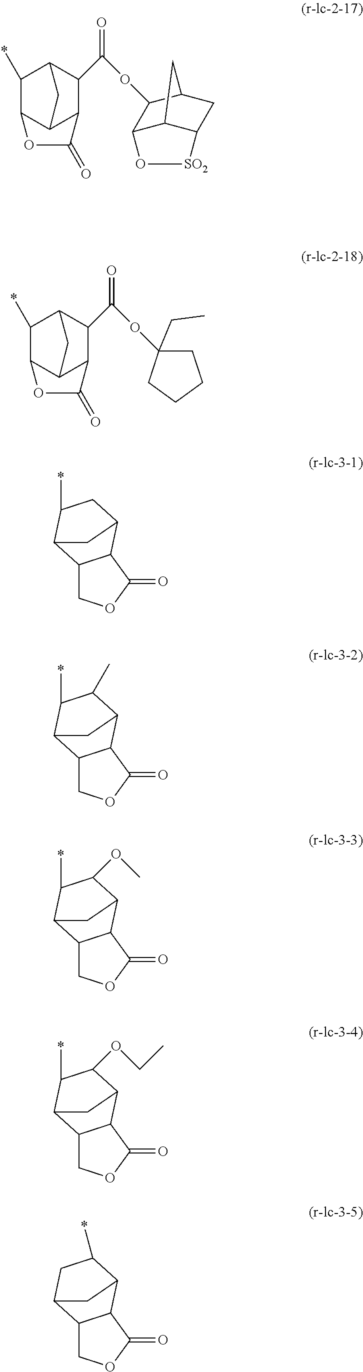

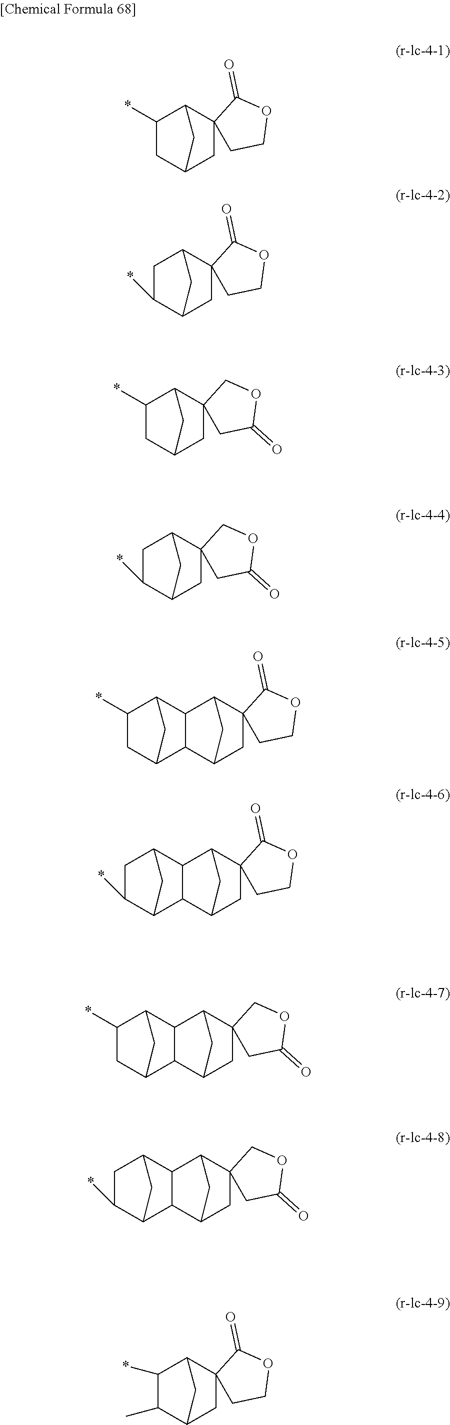

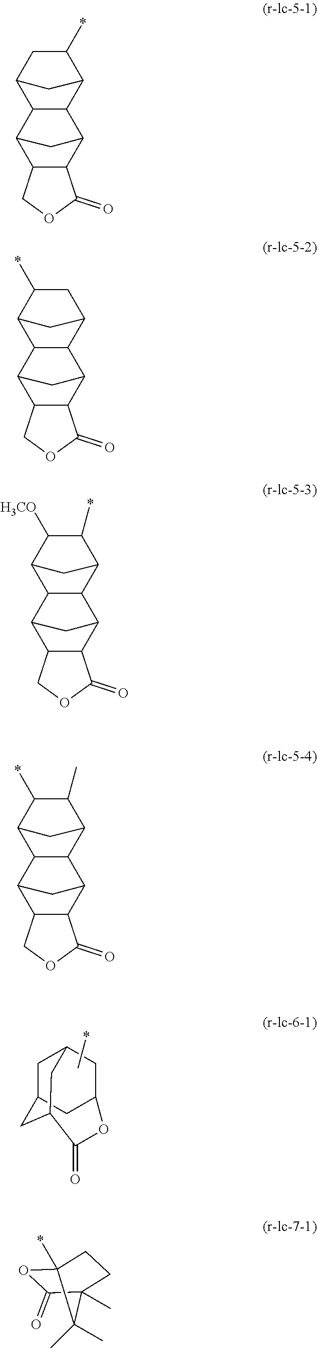

As the constitutional unit (a2), a constitutional unit derived from acrylic acid ester in which the hydrogen atom bonded to the carbon atom at the α-position may be substituted with a substituent is preferable.

It is preferable that such a constitutional unit (a2) is a constitutional unit represented by General Formula (a2-1).

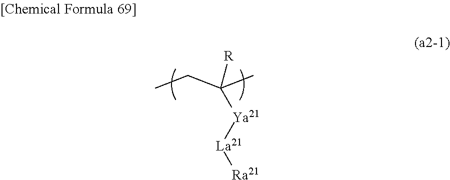

[In the formula, R represents a hydrogen atom, an alkyl group having 1 to 5 carbon atoms, or a halogenated alkyl group having 1 to 5 carbon atoms. Ya²¹ represents a single bond or a divalent linking group. La²¹ represents —O—, —COO—, —CON(R′)—, —OCO—, —CONHCO—, or —CONHCS—, and R′ represents a hydrogen atom or a methyl group. In a case where La²¹ represents —O—, Ya²¹ does not represent —CO—. Ra²¹ represents a lactone-containing cyclic group.]

In Formula (a2-1), R has the same definition as described above. R represents preferably a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a2-1), the divalent linking group as Ya²¹ is not particularly limited, and suitable examples thereof include a divalent hydrocarbon group which may have a substituent and a divalent linking group having heteroatoms.

Examples of the divalent linking group as Ya²¹ include the same groups as those for the divalent linking group as Ya^{x1} in General Formula (a10-1) described above.

It is preferable that Ya²¹ represents a single bond, an ester bond [—C(═O)—O—], an ether bond (—O—), a linear or branched alkylene group, or a combination thereof.

In Formula (a2-1), it is preferable that Ya²¹ represents a single bond and La²¹ represents —COO— or —OCO—.

In Formula (a2-1), Ra²¹ represents a lactone-containing cyclic group.

Suitable examples of the lactone-containing cyclic group as Ra²¹ include groups each represented by General Formulae (a2-r-1) to (a2-r-7).

The constitutional unit (a2) that can be included in the component (A1) may be used alone or two or more kinds thereof.

The component (A1) may or may not have the constitutional unit (a2).

In a case where the component (A1) has the constitutional unit (a2), the proportion of the constitutional unit (a2) is preferably in a range of 1% to 20% by mole, more preferably in a range of 2% to 15% by mole, and still more preferably in a range of 5% to 15% by mole with respect to the total amount (100% by mole) of all constitutional units constituting the component (A1).

In a case where the proportion of the constitutional unit (a2) is set to be greater than or equal to the lower limits of the above-described preferable ranges, the effect to be obtained by allowing the component (A1) to have the constitutional unit (a2) is sufficiently obtained by the above-described effects. Further, in a case where the proportion thereof is set to be less than or equal to the upper limits of the above-described preferable ranges, the constitutional unit (a2) and other constitutional units can be balanced, and the lithography characteristics are improved.

### In Regard to Constitutional Unit (a8):

The constitutional unit (a8) is a constitutional unit derived from a compound represented by General Formula (a8-1).

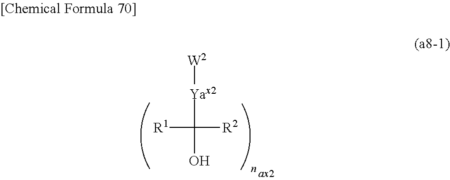

[In the formula, W² represents a polymerizable group-containing group. Ya^{x2} represents a single bond or an (nₐₓ₂+1)-valent linking group. Ya^{x2} and W² may form a condensed ring. R¹ represents a fluorinated alkyl group having 1 to 12 carbon atoms. R² represents an organic group having 1 to 12 carbon atoms which may have a fluorine atom, or a hydrogen atom. R² and Ya^{x2} may be bonded to each other to form a ring structure. nₐₓ2 represents an integer of 1 to 3.]

In Formula (a8-1), the polymerizable group-containing group as W² is the same as described in the section of the polymerizable group-containing group as W⁰¹ in Formula (a01-b).

Suitable examples of the polymerizable group-containing group as W² include a group represented by Chemical Formula: C(R^{X11})(R^{X12})═C(R^{X13})—Ya^{x0}-.

In the chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group.

Examples of the condensed ring formed by Ya^{x2} and W² include a condensed ring formed by a polymerizable group of the W² moiety and by Ya^{x2} and a condensed ring formed by a group other than the polymerizable group of the W² moiety and by Ya^{x2}. The condensed ring formed by Ya^{x2} and W² may have a substituent.

Specific examples of the constitutional unit (a8) are shown below.

In the following formulae, R^{α} represents a hydrogen atom, a methyl group, or a trifluoromethyl group.

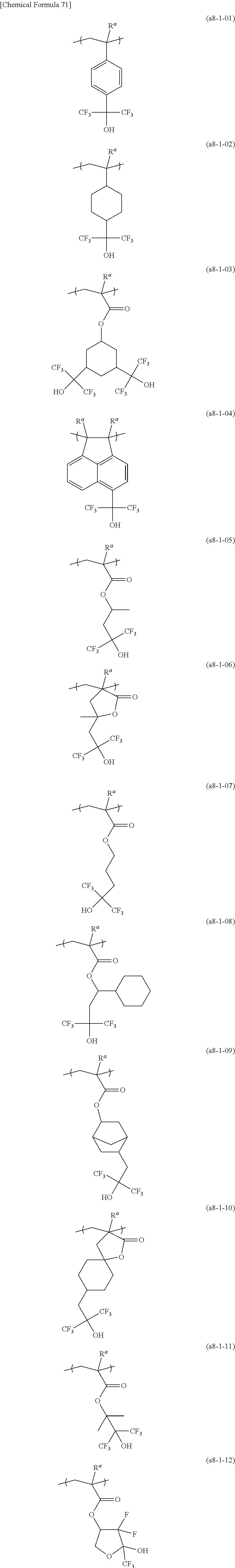

Among the examples, the constitutional unit (a8) is preferably at least one selected from the group consisting of constitutional units each represented by Chemical Formulae (a8-1-01) to (a8-1-04), (a8-1-06), (a8-1-08), (a8-1-09), and (a8-1-10) and more preferably at least one selected from the group consisting of constitutional units each represented by Chemical Formulae (a8-1-01) to (a8-1-04) and (a8-1-09).

The constitutional unit (a8) that can be included in the component (A1) may be used alone or two or more kinds thereof.

The component (A1) may or may not have the constitutional unit (a8).

In a case where the component (A1) has the constitutional unit (a8), the proportion of the constitutional unit (a8) in the component (A1) is preferably 30% by mole or less, more preferably in a range of 0% to 20% by mole, and still more preferably in a range of 0% to 10% by mole with respect to the total amount (100% by mole) of all constitutional units constituting the component (A1).

The component (A1) contained in the resist composition as a base material component may be used alone or in combination with two or more kinds thereof.

In the resist composition, which is a filtration target, the component (A1) contains a copolymer having the constitutional unit (a01) and the constitutional unit (a02) described above, preferably a copolymer having the constitutional unit (a01), the constitutional unit (a02), and the constitutional unit (a1), more preferably a copolymer having the constitutional unit (a01), the constitutional unit (a02), the constitutional unit (a1), and the constitutional unit (a10), and particularly preferably a copolymer having the constitutional unit (a01), the constitutional unit (a02), the constitutional unit (a1), and the constitutional unit (a10).

In the copolymer having the constitutional unit (a01), the constitutional unit (a02), the constitutional unit (a1), and the constitutional unit (a10), the proportion of the constitutional unit (a01) is preferably in a range of 2% to 30% by mole, more preferably in a range of 5% to 25% by mole, and still more preferably in a range of 10% to 20% by mole with respect to the total amount (100% by mole) of all constitutional units constituting the copolymer; the proportion of the constitutional unit (a02) is preferably in a range of 1% to 15% by mole, more preferably in a range of 1% to 10% by mole, and still more preferably in a range of 2% to 8% by mole with respect to the total amount (100% by mole) of all constitutional units constituting the copolymer; the proportion of the constitutional unit (a1) is preferably in a range of 25% to 70% by mole, more preferably in a range of 30% to 60% by mole, and still more preferably in a range of 35% to 55% by mole with respect to the total amount (100% by mole) of all constitutional units constituting the copolymer; and the proportion of the constitutional unit (a10) is preferably in a range of 20% to 50% by mole, more preferably in a range of 25% to 45% by mole, and still more preferably in a range of 30% to 40% by mole with respect to the total amount (100% by mole) of all constitutional units constituting the copolymer.

Here, the total proportion of the constitutional unit (a01), the constitutional unit (a02), the constitutional unit (a1), and the constitutional unit (a10) does not exceed 100% by mole.

The total proportion of the constitutional unit (a01) and the constitutional unit (a02) is preferably in a range of 5% to 40% by mole, more preferably in a range of 10% to 30% by mole, and still more preferably in a range of 15% to 25% by mole with respect to the total amount (100% by mole) of all constitutional units constituting the copolymer.

The ratio of the constitutional unit (a01) to the constitutional unit (a02) is preferably in a range of 60/40 to 90/10, more preferably in a range of 65/35 to 85/15, and still more preferably in a range of 70/30 to 80/20 in terms of the molar ratio expressed as constitutional unit (a01)/constitutional unit (a02).

In a case where the molar ratio thereof is greater than or equal to the lower limits of the above-described preferable ranges, the sensitivity in a case of forming a resist pattern is further improved. Meanwhile, in a case where the molar ratio thereof is less than or equal to the upper limits of the above-described preferable ranges, lithography characteristics such as the reduction of the roughness in a pattern are further improved.

The weight-average molecular weight (Mw) (in terms of polystyrene according to gel permeation chromatography (GPC)) of the component (A1) is not particularly limited, but is preferably in a range of 1000 to 50000, more preferably in a range of 2000 to 30000, and still more preferably in a range of 3000 to 20000.

In a case where the Mw of the component (A1) is less than or equal to the upper limits of the above-described preferable ranges, the resist composition exhibits a satisfactory solubility in a resist solvent for a resist enough to be used as a resist. On the contrary, in a case where the Mw of the component (A1) is greater than or equal to the lower limits of the above-described preferable ranges, the dry etching resistance and the cross-sectional shape of the resist pattern are excellent.

Further, the dispersity (Mw/Mn) of the component (A1) is not particularly limited, but is preferably in a range of 1.0 to 4.0, more preferably in a range of 1.0 to 3.0, and particularly preferably in a range of 1.0 to 2.0. Further, Mn represents the number average molecular weight.

### In Regard to Component (A2)

The resist composition, which is the filtration target, may use, as the component (A), a base material component (hereinafter, referred to as “component (A2)”) whose solubility in the developing solution is changed by the action of an acid, which does not correspond to the above-described component (A1), in combination.

The component (A2) is not particularly limited and may be optionally selected from a plurality of components of the related art which have been known as base material components for a chemically amplified resist composition and used.

As the component (A2), a polymer compound or a low-molecular-weight compound may be used alone or in combination of two or more kinds thereof.

The proportion of the component (A1) in the component (A) is preferably 25% by mass or greater, more preferably 50% by mass or greater, and still more preferably 75% by mass or greater, and may be 100% by mass with respect to the total mass of the component (A). In a case where the proportion thereof is 25% by mass or greater, a resist pattern having various excellent lithography characteristics such as high sensitivity, resolution, and reduction of roughness is likely to be formed, and in addition, a process margin is also likely to be widened.

The content of the component (A) in the resist composition may be adjusted depending on the resist film thickness to be formed and the like.

### <<Organic Solvent Component (S)>>

The resist composition, which is the filtration target, contains an organic solvent component (hereinafter, referred to as “component (S)”). Such a resist composition is produced by dissolving the resist material in the component (S). The component (S) may be any organic solvent which can dissolve each component to be used to obtain a uniform solution, and an optional organic solvent can be appropriately selected from those which have been known as solvents of a chemically amplified resist composition and then used.

In the resist composition, the component (S) may be used alone or in the form of a mixed solvent of two or more kinds thereof. Among these, PGMEA, PGME, γ-butyrolactone, EL, or cyclohexanone is preferable.

Further, a mixed solvent obtained by mixing PGMEA with a polar solvent is also preferable as the component (S). The blending ratio (mass ratio) may be appropriately determined in consideration of the compatibility between PGMEA and the polar solvent. Further, a mixed solvent of γ-butyrolactone and at least one selected from PGMEA and EL is also preferable as the component (S). In this case, as the mixing ratio, the mass ratio between the former and the latter is preferably in a range of 70:30 to 95:5.

The amount of the component (S) to be used is not particularly limited and is appropriately set to have a concentration which enables coating a substrate or the like depending on the thickness of the coated film. The component (S) is typically used in an amount such that the solid content concentration of the resist composition is set to be in a range of 0.1% to 20% by mass and preferably in a range of 0.2% to 15% by mass.

### <<Other Component>>

The resist composition, which is the filtration target, may further contain other components in addition to the above-described component (A). Examples of the other components include a component (B), a component (D), a component (E), and a component (F) described below.

### Acid Generator Component (B)

The resist composition, which is the filtration target, may further contain an acid generator component (B) (component (B)) that generates an acid upon light exposure. The component (B) is not particularly limited, and those which have been suggested so far as an acid generator for a chemically amplified resist composition in the related art can be used.

Examples of the acid generation agent include various acid generation agents, for example, onium salt-based acid generation agents such as iodonium salts and sulfonium salts; oxime sulfonate-based acid generation agents; diazomethane-based acid generation agents such as bisalkyl or bisaryl sulfonyl diazomethanes and poly(bis-sulfonyl)diazomethanes; nitrobenzyl sulfonate-based acid generation agents, iminosulfonate-based acid generation agents, and disulfone-based acid generation agents.

Examples of the onium salt-based acid generation agents include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

Examples of the onium salt-based acid generation agents include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

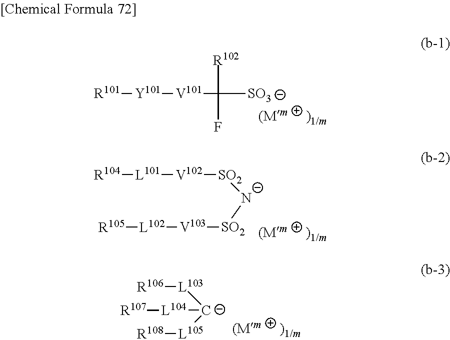

[In the formulae, R¹⁰¹ and R¹⁰⁴ to R¹⁰⁸ each independently represent a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group containing an oxygen atom or a single bond. V¹⁰¹ to V¹⁰³ each independently represent a single bond, an alkylene group, or a fluorinated alkylene group. Here, Y¹⁰¹ and V¹⁰¹ do not represent a single bond at the same time. L¹⁰¹ and L¹⁰² each independently represent a single bond or an oxygen atom. L¹⁰³ to L¹⁰⁵ each independently represent a single bond, —CO—, or —SO₂—. m represents an integer of 1 or greater, and M′^{m+} represents an m-valent onium cation.]

### {Anion Moiety}

Anions in Component (b-1)

In Formula (b-1), R¹⁰¹ represents a cyclic group which may have a substituent, a chain-like alkyl group which may have a substituent, or a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. In addition, it is preferable that the aliphatic hydrocarbon group is saturated.

The aromatic hydrocarbon group as R¹⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring of the aromatic hydrocarbon group as R¹⁰¹ include benzene, fluorene, naphthalene, anthracene, phenanthrene, biphenyl, or an aromatic heterocyclic ring in which some carbon atoms constituting any of these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as R¹⁰¹ include a group obtained by removing one hydrogen atom from the aromatic ring (an aryl group such as a phenyl group or a naphthyl group) and a group in which one hydrogen atom of the aromatic ring is substituted with an alkylene group (for example, a benzyl group, a phenethyl group, or a 1-naphthylmethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R¹⁰¹ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclodecane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R¹⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is still more preferable, and an adamantyl group is particularly preferable.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable. Specific examples thereof include a methylene group [—CH₂—], an ethylene group [—(CH₂)₂-], a trimethylene group [—(CH₂)₃-], a tetramethylene group [—(CH₂)₄-], and a pentamethylene group [—(CH₂)₅-].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms. As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Further, the cyclic hydrocarbon group as R¹⁰¹ may have a heteroatom such as a heterocyclic ring. Specific examples thereof include lactone-containing cyclic groups each represented by General Formulae (a2-r-1) to (a2-r-7), —SO₂-containing cyclic groups each represented by General Formulae (b5-r-1) to (b5-r-4), and other heterocyclic groups each represented by Chemical Formulae (r-hr-1) to (r-hr-16). In the formulae, * represents a bonding site with respect to Y¹⁰¹ in Formula (b-1).

Examples of the substituent for the cyclic group as R¹⁰¹ include an alkyl group, an alkoxy group, a halogen atom, a halogenated alkyl group, a hydroxyl group, a carbonyl group, and a nitro group. An alkyl group having 1 to 5 carbon atoms is preferable as the alkyl group serving as a substituent.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

A fluorine atom, a bromine atom, or an iodine atom is preferable as the halogen atom serving as a substituent.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group are substituted with the above-described halogen atoms.

The carbonyl group as the substituent is a group that substitutes a methylene group (—CH₂—) constituting the cyclic hydrocarbon group.

The cyclic hydrocarbon group as R¹⁰¹ may be a condensed cyclic group containing a condensed ring in which an aliphatic hydrocarbon ring and an aromatic ring are condensed. Examples of the condensed ring include those obtained by fusing one or more aromatic rings with a polycycloalkane having a crosslinked ring-based polycyclic skeleton. Specific examples of the crosslinked ring polycycloalkane include a bicycloalkane such as bicyclo[2.2.1]heptane (norbornane) and bicyclo[2.2.2]octane. As the condensed cyclic group, a group having a condensed ring in which two or three aromatic rings are condensed with a bicycloalkane is preferable, and a group having a condensed ring in which two or three aromatic rings are condensed with bicyclo[2.2.2]octane is more preferable. Specific examples of the condensed cyclic group as R¹⁰¹ include those represented by Formulae (r-br-1) and (r-br-2). In the formulae, * represents a bonding site with respect to Y¹⁰¹ in Formula (b-1).

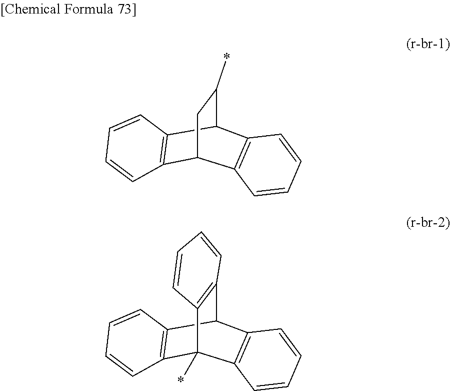 [SMILES: *C1CC2c3ccccc3C1c1ccccc12 ; *C12c3ccccc3C(c3ccccc31)c1ccccc12]

Examples of the substituent that the condensed cyclic group as R¹⁰¹ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the condensed cyclic group include those exemplified as the substituent of the cyclic group as R¹⁰¹.

Examples of the aromatic hydrocarbon group as the substituent of the condensed cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and a heterocyclic group represented by any of Formulae (r-hr-1) to (r-hr-6).

Examples of the alicyclic hydrocarbon group as the substituent of the condensed cyclic group include a group in which one hydrogen atom has been removed from a monocycloalkane such as cyclopentane or cyclohexane, a group in which one hydrogen atom has been removed from a polycycloalkane such as adamantane, norbornane, isobornane, tricyclodecane, or tetracyclododecane, a lactone-containing cyclic group represented by any of General Formulae (a2-r-1) to (a2-r-7), a —SO₂-containing cyclic group represented by any of General Formulae (b5-r-1) to (b5-r-4), and a heterocyclic group represented by any of Formulae (r-hr-7) to (r-hr-16).

Chain-Like Alkyl Group which May have Substituent:

The chain-like alkyl group as R¹⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R¹⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (allyl group), and a butynyl group. Examples of the branched alkenyl group include a 1-methylvinyl group, a 2-methylvinyl group, a 1-methylpropenyl group, and a 2-methylpropenyl group. Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹⁰¹.

In Formula (b-1), Y¹⁰¹ represents a single bond or a divalent linking group having an oxygen atom.

In a case where Y¹⁰¹ represents a divalent linking group having an oxygen atom, Y¹⁰¹ may have an atom other than the oxygen atom. Examples of atoms other than an oxygen atom include a carbon atom, a hydrogen atom, a sulfur atom, and a nitrogen atom.

Examples of the divalent linking group having an oxygen atom include linking groups each represented by General Formulae (y-al-1) to (y-al-7). In Formulae (y-al-1) to (y-al-7), V′¹⁰¹ in Formulae (y-al-1) to (y-al-7) is bonded to R¹⁰¹ in Formula (b-1).

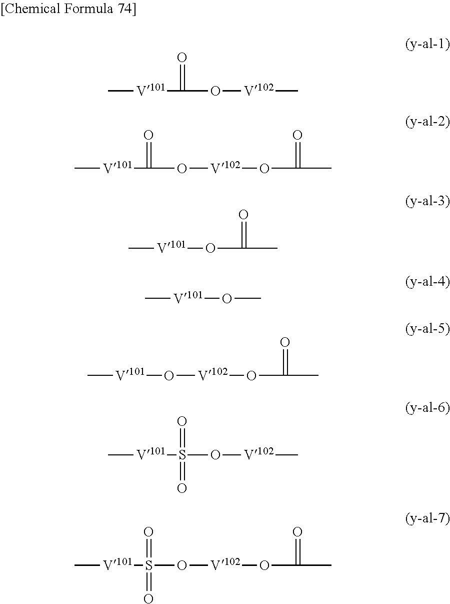

[In the formulae, V′¹⁰¹ represents a single bond or an alkylene group having 1 to 5 carbon atoms, and V′¹⁰² represents a divalent saturated hydrocarbon group having 1 to 30 carbon atoms.]

As the divalent saturated hydrocarbon group as V′¹⁰², an alkylene group having 1 to 30 carbon atoms is preferable, an alkylene group having 1 to 10 carbon atoms is more preferable, and an alkylene group having 1 to 5 carbon atoms is still more preferable.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)₂CH₂—, or —CH(CH₂CH₃)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—].

Further, a part of methylene group in the alkylene group as V′¹⁰¹ and V′¹⁰² may be substituted with a divalent aliphatic cyclic group having 5 to 10 carbon atoms. As the aliphatic cyclic group, a divalent group in which one hydrogen atom has been removed from the cyclic aliphatic hydrocarbon group (a monocyclic aliphatic hydrocarbon group or a polycyclic aliphatic hydrocarbon group) as Ra′³ in Formula (a1-r-1) is preferable, and a cyclohexylene group, a 1,5-adamantylene group, or a 2,6-adamantylene group is more preferable.

In Formula (b-1), V¹⁰¹ represents a single bond, an alkylene group, or a fluorinated alkylene group. Among these, it is preferable that V¹⁰¹ represents a single bond or a linear fluorinated alkylene group having 1 to 4 carbon atoms.

In Formula (b-1), R¹⁰² represents a fluorine atom or a fluorinated alkyl group having 1 to 5 carbon atoms. R¹⁰² represents preferably a fluorine atom or a perfluoroalkyl group having 1 to 5 carbon atoms and more preferably a fluorine atom.

In a case where Y¹⁰¹ represents a single bond, specific example of the anion moiety represented by Formula (b-1) include a fluorinated alkylsulfonate anion such as a trifluoromethanesulfonate anion or a perfluorobutanesulfonate anion. Further, in a case where Y¹⁰¹ represents a divalent linking group having an oxygen atom, specific examples thereof include an anion represented by any of Formulae (an-1) to (an-3).

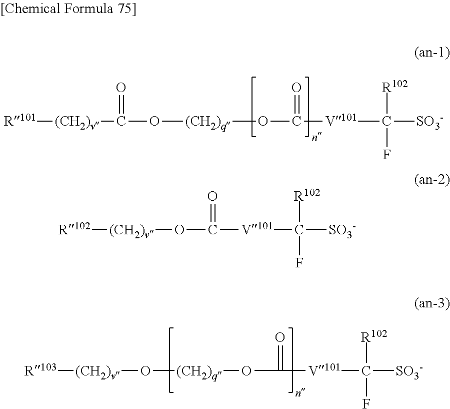

[In the formulae, R″¹⁰¹ represents an aromatic cyclic group which may have a substituent, a monovalent heterocyclic group represented by any of Chemical Formulae (r-hr-1) to (r-hr-6), a condensed cyclic group represented by Formula (r-br-1) or (r-br-2), a chain-like alkyl group which may have a substituent, or an aromatic cyclic group which may have a substituent. R″¹⁰² represents an aliphatic cyclic group which may have a substituent, a condensed cyclic group represented by Formula (r-br-1) or (r-br-2), a lactone-containing cyclic group represented by any of General Formulae (a2-r-1) and (a2-r-3) to (a2-r-7), or a —SO₂-containing cyclic group represented by any of General Formulae (b5-r-1) to (b5-r-4). R″¹⁰³ represents an aromatic cyclic group which may have a substituent, an aliphatic cyclic group which may have a substituent, or a chain-like alkenyl group which may have a substituent. V″¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v″ independently represents an integer of 0 to 3, each q″ independently represents an integer of 0 to 20, and n″ represents 0 or 1.]

As the aliphatic cyclic group as R″¹⁰¹, R″¹⁰², and R″¹⁰³ which may have a substituent, the same groups as those for the cyclic aliphatic hydrocarbon group as R¹⁰¹ in Formula (b-1) are preferable. Examples of the substituent include the same groups as those for the substituent which may substitute the cyclic aliphatic hydrocarbon group as R¹⁰¹ in Formula (b-1).

As the aromatic cyclic group which may have a substituent as R″¹⁰¹ and R″¹⁰³, the groups described as the aromatic hydrocarbon group in the cyclic hydrocarbon group as R¹⁰¹ in Formula (b-1) are preferable. Examples of the substituent include the same groups as those for the substituent which may substitute the aromatic hydrocarbon group as R¹⁰¹ in Formula (b-1).

As the chain-like alkyl group as R″¹⁰¹ which may have a substituent, the same groups as those for the chain-like alkyl group as R¹⁰¹ in Formula (b-1) are preferable.

As the chain-like alkenyl group as R″¹⁰³ which may have a substituent, the same groups as those for the chain-like alkenyl group as R¹⁰¹ in Formula (b-1) are preferable.

Anions in component (b-2) In Formula (b-2), R¹⁰⁴ and R¹⁰⁵ each independently represent a cyclic group which may have a substituent, a chain-like alkyl group which may have a substituent, or a chain-like alkenyl group which may have a substituent, and examples thereof include those for R¹⁰¹ in Formula (b-1). Here, R¹⁰⁴ and R¹⁰⁵ may be bonded to each other to form a ring.

R¹⁰⁴ and R¹⁰⁵ represent preferably a chain-like alkyl group which may have a substituent and more preferably a linear or branched alkyl group or a linear or branched fluorinated alkyl group.

The chain-like alkyl group has preferably 1 to 10 carbon atoms, more preferably 1 to 7 carbon atoms, and still more preferably 1 to 3 carbon atoms. It is preferable that the number of carbon atoms in the chain-like alkyl group as R¹⁰⁴ and R¹⁰⁵ decreases within the range of the number of carbon atoms because the solubility in a solvent for a resist is also satisfactory. Further, in the chain-like alkyl group as R¹⁰⁴ and R¹⁰⁵, it is preferable that the number of hydrogen atoms substituted with fluorine atoms is as large as possible because the acid strength increases and the transparency to high energy light with a wavelength of 250 nm or less or electron beams is improved. The proportion of fluorine atoms in the chain-like alkyl group, that is, the fluorination ratio is preferably in a range of 70% to 100% and more preferably in a range of 90% to 100%, and it is most preferable that the chain-like alkyl group is a perfluoroalkyl group in which all hydrogen atoms are substituted with fluorine atoms.

In Formula (b-2), V¹⁰² and V¹⁰³ each independently represent a single bond, an alkylene group, or a fluorinated alkylene group, and examples thereof include the same groups as those for V¹⁰¹ in Formula (b-1).

In Formula (b-2), L¹⁰¹ and L¹⁰² each independently represent a single bond or an oxygen atom.

Anions in component (b-3) In Formula (b-3), R¹⁰⁶ to R¹⁰⁸ each independently represent a cyclic group which may have a substituent, a chain-like alkyl group which may have a substituent, or a chain-like alkenyl group which may have a substituent, and examples thereof include those for R¹⁰¹ in Formula (b-1).

In Formula (b-3), L¹⁰³ to L¹⁰⁵ each independently represent a single bond, —CO—, or —SO₂—.

Among these, as the anion moiety of the component (B), an anion of the component (b-1) is preferable, and an anion represented by Formula (an-1) is more preferable.

### {Cation Moiety}

In Formulae (b-1), (b-2), and (b-3), M′^{m+} represents an m-valent onium cation. m and M′^{m+} each have the same definition as that for m and M^{m+} in General Formula (a01-b).

Among these, a sulfonium cation and an iodonium cation are preferable. m represents an integer of 1 or greater.

Among the examples, as the cation moiety of the component (B), a sulfonium cation is preferable, a cation represented by any of Formulae (ca-1) to (ca-3) is more preferable, a cation represented by Formula (ca-1) is still more preferable, and a cation represented by any of Formulae (ca-1-1) to (ca-1-87) is particularly preferable.

In the resist composition, the component (B) may be used alone or in a combination of two or more kinds thereof.

The content of the component (B) in the resist composition is preferably less than 50 parts by mass, more preferably in a range of 0 to 40 parts by mass, still more preferably in a range of 0 to 30 parts by mass, and still more preferably in a range of 0 to 20 parts by mass with respect to 100 parts by mass of the component (A1).

In a case where the content of the component (B) is set to be in the above-described preferable ranges, pattern formation can be sufficiently carried out. Further, it is preferable that each component of the resist composition is dissolved in an organic solvent from the viewpoint that a uniform solution is easily obtained and the storage stability of the resist composition is improved.

### Base Component (D)

The resist composition, which is the filtration target, may further contain a base component (component (D)) that traps an acid generated upon light exposure (that is, controls the diffusion of an acid) in addition to component (A). The component (D) acts as a quencher (an acid diffusion control agent) which traps the acid generated in the resist composition upon light exposure.

Examples of the component (D) include a photodecomposable base (D1) having an acid diffusion controllability (hereinafter, referred to as “component (D1)”) which is lost by the decomposition upon light exposure and a nitrogen-containing organic compound (D2) (hereinafter, referred to as “component (D2)”) which does not correspond to the component (D1). Among these, the photodecomposable base (component (D1)) is preferable from the viewpoint of easily increasing the sensitivity, reducing the roughness, and improving the characteristic of suppressing occurrence of coating defects. The compound described as the component (D1) below may be used as the acid generator component (component (B)) depending on the combination with other compounds.

### In Regard to Component (D1)

In a case where a resist composition containing the component (D1) is obtained, the contrast between an exposed portion and an unexposed portion of the resist film can be further improved in a case of forming a resist pattern. The component (D1) is not particularly limited as long as the component is decomposed upon light exposure and loses acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1-3)”) are preferable.

Since the components (d1-1) to (d1-3) are decomposed and lose the acid diffusion controllability (basicity), the components (d1-1) to (d1-3) do not function as a quencher at the exposed portion of the resist film, but function as a quencher at the unexposed portion of the resist film.

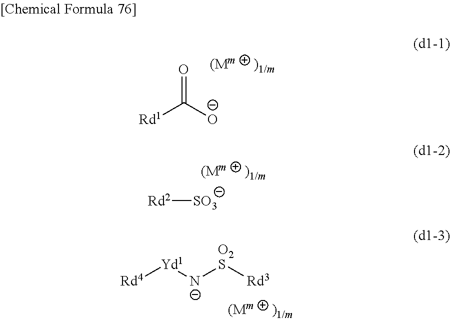

[In the formulae, Rd¹ to Rd⁴ represent a cyclic group which may have a substituent, a chain-like alkyl group which may have a substituent, or a chain-like alkenyl group which may have a substituent. Here, the carbon atom adjacent to the S atom as Rd² in Formula (d1-2) has no fluorine atom bonded thereto. Yd¹ represents a single bond or a divalent linking group. m represents an integer of 1 or greater, and each M^{m+} independently represents an m-valent organic cation.]

{Component (d1-1)}

### Anion Moiety

In Formula (d1-1), Rd¹ represents a cyclic group which may have a substituent, a chain-like alkyl group which may have a substituent, or a chain-like alkenyl group which may have a substituent, and examples thereof include the same groups as those for R′²⁰¹.

Among these, it is preferable that the group as Rd¹ represents an aromatic hydrocarbon group which may have a substituent, an aliphatic cyclic group which may have a substituent, or a chain-like alkyl group which may have a substituent. Examples of the substituent that may be included in these groups include a hydroxyl group, an oxo group, an alkyl group, an aryl group, a fluorine atom, a fluorinated alkyl group, a lactone-containing cyclic group represented by any of Formulae (a2-r-1) to (a2-r-7), an ether bond, an ester bond, and a combination thereof. In a case where an ether bond or an ester bond is included as the substituent, the substituent may be bonded through an alkylene group, and a linking group represented by any of Formulae (y-al-1) to (y-al-5) is preferable as the substituent. Further, in a case where the aromatic hydrocarbon group, the aliphatic cyclic group, or the chain-like alkyl group, as Rd¹, has a linking group represented by each of General Formulae (y-al-1) to (y-al-7) as a substituent, in General Formulae (y-al-1) to (y-al-7), the group that is bonded to a carbon atom constituting the aromatic hydrocarbon group, the aliphatic cyclic group, or the chain-like alkyl group, as Rd¹, in General Formula (d1-1) is V′¹⁰¹ in General Formulae (y-al-1) to (y-al-7).

Suitable examples of the aromatic hydrocarbon group include a phenyl group, a naphthyl group, and a polycyclic structure having a bicyclooctane skeleton (for example, a polycyclic structure formed of a bicyclooctane skeleton and a ring structure other than the bicyclooctane skeleton).

As the aliphatic cyclic group, a group in which one or more hydrogen atoms have been removed from a polycycloalkane such as adamantane, norbornane, isobornane, tricyclodecane or tetracyclododecane is more preferable.

It is preferable that the chain-like alkyl group has 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group has preferably 1 to 11 carbon atoms, more preferably 1 to 8 carbon atoms, and still more preferably 1 to 4 carbon atoms. The fluorinated alkyl group may have an atom other than a fluorine atom. Examples of the atom other than a fluorine atom include an oxygen atom, a sulfur atom, and a nitrogen atom.

Specific preferred examples of the anion moiety in the component (d1-1) are described below.

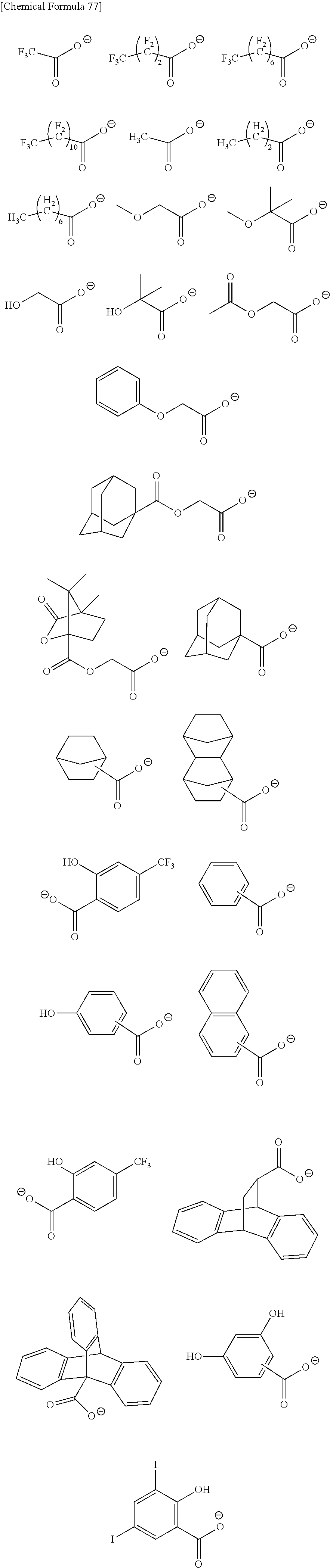

### Cation Moiety

In Formula (d1-1), M^{m+} represents an m-valent organic cation.

Examples of the organic cation as M^{m+} include the same cations as those for the cations each represented by Formulae (ca-1) to (ca-3). Among these, a cation represented by General Formula (ca-1) is more preferable, and cations each represented by Formulae (ca-1-1) to (ca-1-87) are still more preferable.

The component (di-1) may be used alone or in combination of two or more kinds thereof.

{Component (d1-2)}

### Anion Moiety

In Formula (d1-2), Rd² represents a cyclic group which may have a substituent, a chain-like alkyl group which may have a substituent, or a chain-like alkenyl group which may have a substituent, and examples thereof include the same groups as those for R′²⁰¹.

Here, the carbon atom adjacent to the S atom in Rd² has no fluorine atom bonded thereto (the carbon atom is not substituted with a fluorine atom). In this manner, the anion of the component (d1-2) becomes an appropriately weak acid anion, thereby improving the quenching ability of the component (D).

It is preferable that Rd² represents a chain-like alkyl group which may have a substituent or an aliphatic cyclic group which may have a substituent. The chain-like alkyl group has preferably 1 to 10 carbon atoms and more preferably 3 to 10 carbon atoms. As the aliphatic cyclic group, a group in which one or more hydrogen atoms have been removed from adamantane, norbornane, isobornane, tricyclodecane, or tetracyclododecane (a group which may have a substituent); and a group in which one or more hydrogen atoms have been removed from camphor are more preferable.

The hydrocarbon group as Rd² may have a substituent, and examples of the substituent include the same groups as those for the substituent which may be included in the hydrocarbon group (such as an aromatic hydrocarbon group, an aliphatic cyclic group, or a chain-like alkyl group) as Rd¹ in Formula (d1-1).

Specific preferred examples of the anion moiety in the component (d1-2) are described below.

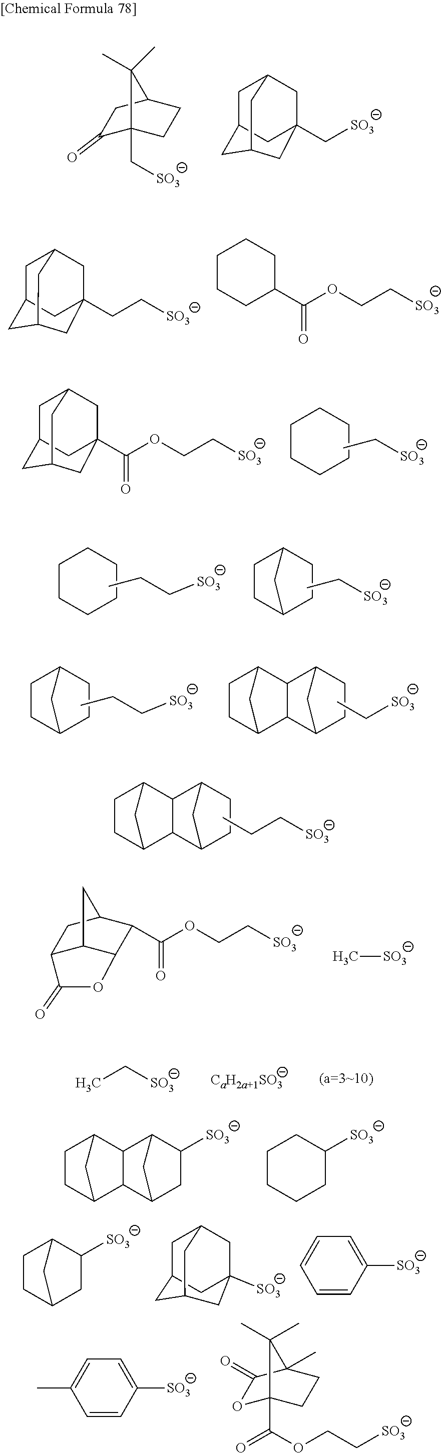

### Cation Moiety

In Formula (d1-2), M^{m+} represents an m-valent organic cation and has the same definition as that for M^{m+} in Formula (d1-1).

The component (d1-2) may be used alone or in combination of two or more kinds thereof.

{Component (d1-3)}

### Anion Moiety

In Formula (d1-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ are more preferable.

In Formula (d1-3), Rd⁴ represents a cyclic group which may have a substituent, a chain-like alkyl group which may have a substituent, or a chain-like alkenyl group which may have a substituent, and examples thereof include the same groups as those for R′²⁰¹.

Among these, an alkyl group which may have a substituent, an alkoxy group which may have a substituent, an alkenyl group which may have a substituent, or a cyclic group which may have a substituent is preferable.

It is preferable that the alkyl group as Rd⁴ is a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ is an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may have an alkyl group having 1 to 5 carbon atoms or a halogenated alkyl group having 1 to 5 carbon atoms as a substituent.

Examples of the cyclic group as Rd⁴ include the same groups as those for the cyclic group as R′²⁰¹. Among these, an alicyclic group in which one or more hydrogen atoms have been removed from a cycloalkane such as cyclopentane, cyclohexane, adamantane, norbornane, isobornane, tricyclodecane or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are improved. Further, in a case where Rd⁴ represents an aromatic group, the resist composition has excellent light absorption efficiency in lithography using EUV or the like as an exposure light source, and thus the sensitivity and lithography characteristics are improved.

In Formula (d1-3), Yd¹ represents a single bond or a divalent linking group.

The divalent linking group as Yd¹ is not particularly limited, and examples thereof include a divalent hydrocarbon group (an aliphatic hydrocarbon group or an aromatic hydrocarbon group) which may have a substituent and a divalent linking group having a heteroatom. These divalent linking groups are the same as those for the divalent hydrocarbon group which may have a substituent and the divalent linking group having a heteroatom described in the section of the divalent linking group as Ya²¹ in Formula (a2-1).

It is preferable that Yd¹ represents a carbonyl group, an ester bond, an amide bond, an alkylene group, or a combination of these. As the alkylene group, a linear or branched alkylene group is more preferable, and a methylene group or an ethylene group is still more preferable.

Specific preferred examples of the anion moiety in the component (d1-3) are described below.

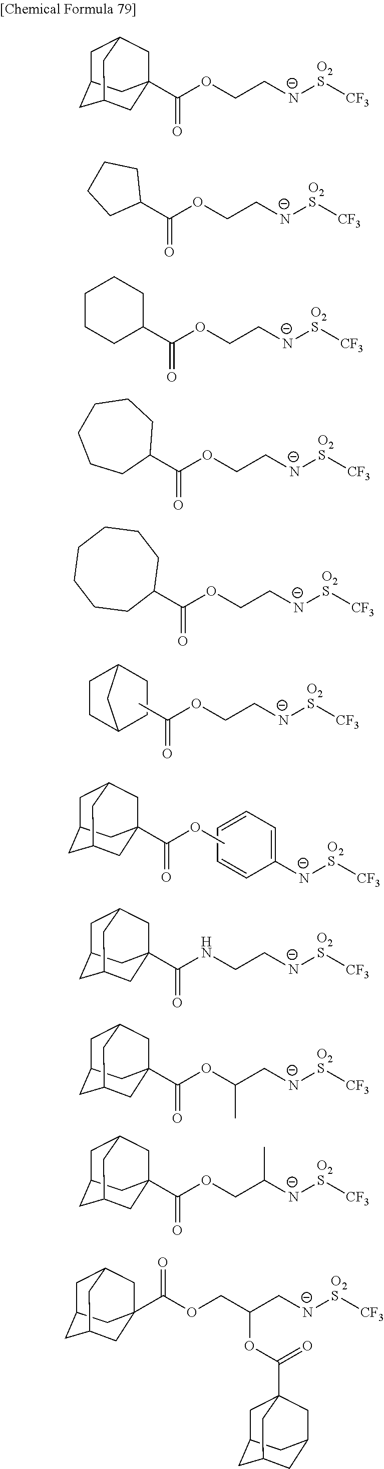

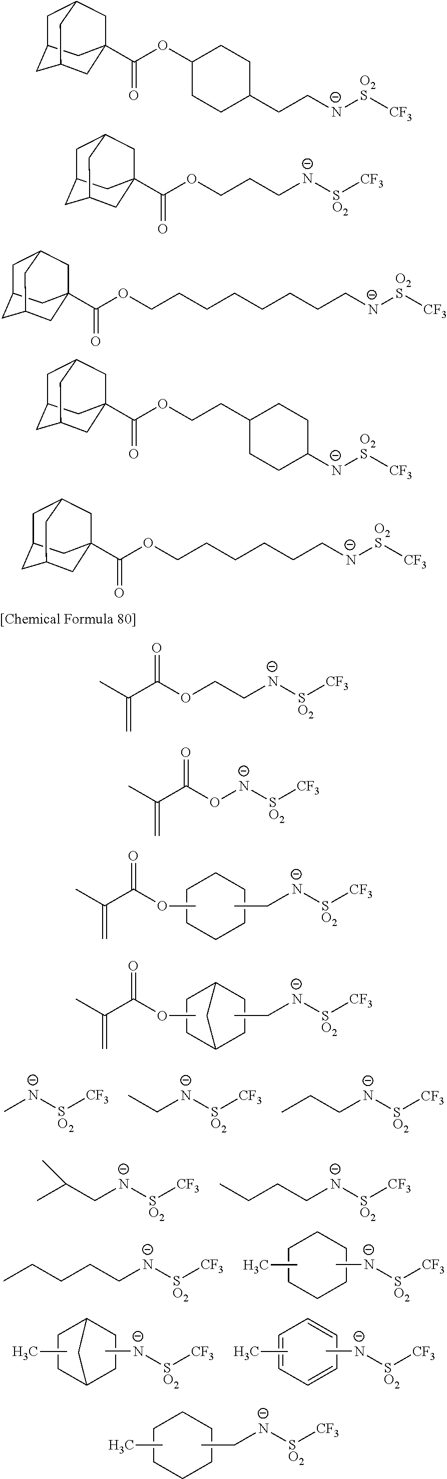

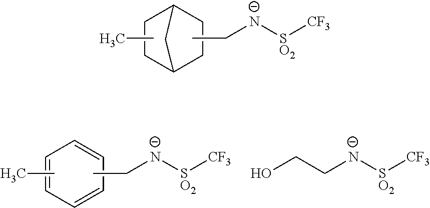

### Cation Moiety

In Formula (d1-3), M^{m+} represents an m-valent organic cation and has the same definition as that for M^{m+} in Formula (d1-1).

The component (d1-3) may be used alone or in combination of two or more kinds thereof.

As the component (D1), only one of the above-described components (d1-1) to (d1-3) or a combination of two or more kinds thereof may be used.

In a case where the resist composition contains the component (D1), the content of the component (D1) in the resist composition is preferably in a range of 0.5 to 20 parts by mass, more preferably in a range of 1 to 15 parts by mass, and still more preferably in a range of 2 to 10 parts by mass with respect to 100 parts by mass of the component (A1).

In a case where the content of the component (D1) is greater than or equal to the lower limits of the above-described preferable ranges, satisfactory lithography characteristics and a satisfactory resist pattern shape are likely to be obtained. On the contrary, in a case where the content is less than or equal to the upper limits of the above-described ranges, the sensitivity can be satisfactorily maintained and the throughput is also excellent.

### Method of Producing Component (D1):

The methods of producing the component (d1-1) and the component (d1-2) are not particularly limited, and these components can be produced by known methods. Further, the method of producing the component (d1-3) is not particularly limited, and the component is produced by the same method as disclosed in United States Patent Application, Publication No. 2012-0149916.

### In Regard to Component (D2)

The component (D) may contain a nitrogen-containing organic compound component (hereinafter, referred to as “component (D2)”) that does not correspond to the component (D1) described above.

The component (D2) is not particularly limited as long as the component functions as an acid diffusion control agent and does not correspond to the component (D1), and an optional component may be selected from known components and then used. Among the examples, an aliphatic amine is preferable, and particularly a secondary aliphatic amine and a tertiary aliphatic amine are more preferable.

The aliphatic amine is an amine containing one or more aliphatic groups, and the number of carbon atoms in the aliphatic group is preferably in a range of 1 to 12.

Examples of these aliphatic amines include amines in which at least one hydrogen atom of ammonia NH₃ has been substituted with an alkyl group or hydroxyalkyl group having 12 or less carbon atoms (alkylamines or alkylalcoholamines), and cyclic amines.

Specific examples of the alkylamines and the alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines such as diethanolamine, triethanolamine, diisopropanolamine, triisopropanolamine, di-n-octanolamine, and tri-n-octanolamine. Among these, a trialkylamine having 6 to 30 carbon atoms is still more preferable, and tri-n-pentylamine or tri-n-octylamine is particularly preferable.

Examples of the cyclic amine include a heterocyclic compound having a nitrogen atom as a heteroatom. The heterocyclic compound may be a monocyclic compound (aliphatic monocyclic amine) or a polycyclic compound (aliphatic polycyclic amine).

Specific examples of the aliphatic monocyclic amine include piperidine and piperazine. The aliphatic polycyclic amine preferably has 6 to 10 carbon atoms, and specific examples thereof include 1, 5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxy}ethyl]amine, and triethanolamine triacetate. Among these, triethanolamine triacetate is preferable.

As the component (D2), an aromatic amine may be used.

Examples of aromatic amines include 4-dimethylaminopyridine, pyrrole, indole, pyrazole, imidazole, and derivatives thereof, tribenzylamine, 2,6-diisopropylaniline, N-tert-butoxycarbonylpyrrolidine, and 2,6-di-tert-butylpyridine.

The component (D2) may be used alone or in combination of two or more kinds thereof. In a case where the resist composition contains the component (D2), the content of the component (D2) in the resist composition is typically in a range of 0.01 to 5 parts by mass with respect to 100 parts by mass of the component (A1). In a case where the content thereof is set to be in the above-described range, the resist pattern shape, the post exposure temporal stability, and the like are improved.

At least one compound (E) selected from group consisting of organic carboxylic acid, phosphorus oxo acid, and derivative thereof.

For the purpose of preventing deterioration of sensitivity and improving the resist pattern shape and the post-exposure temporal stability, the resist composition which is the filtration target may contain, as an optional component, at least one compound (E) (hereinafter, referred to as “component (E)”) selected from the group consisting of an organic carboxylic acid, a phosphorus oxo acid, and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

Examples of the phosphorus oxo acid derivative include an ester obtained by substituting a hydrogen atom in the above-described oxo acid with a hydrocarbon group. Examples of the hydrocarbon group include an alkyl group having 1 to 5 carbon atoms and an aryl group having 6 to 15 carbon atoms.

Examples of the phosphoric acid derivatives include phosphoric acid esters such as phosphoric acid di-n-butyl ester and phosphoric acid diphenyl ester.

Examples of the phosphonic acid derivatives include phosphonic acid esters such as phosphonic acid dimethyl ester, phosphonic acid di-n-butyl ester, phenylphosphonic acid, phosphonic acid diphenyl ester, and phosphonic acid dibenzyl ester.

Examples of the phosphinic acid derivatives include phosphinic acid ester and phenylphosphinic acid.

In the resist composition, the component (E) may be used alone or in a combination of two or more kinds thereof.

In a case where the resist composition contains the component (E), the content of the component (E) is preferably in a range of 0.01 to 5 parts by mass and more preferably in a range of 0.05 to 3 parts by mass with respect to 100 parts by mass of the component (A1). In a case where the content thereof is in the above-described range, the sensitivity, lithography characteristics, and the like are improved.

### Fluorine Additive Component (F)

The resist composition, which is the filtration target, may contain a fluorine additive component (hereinafter, referred to as “component (F)”) as a hydrophobic resin. The component (F) is used to impart water repellency to the resist film and used as a resin different from the component (A), whereby the lithography characteristics can be improved.

As the component (F), for example, the fluorine-containing polymer compounds described in Japanese Unexamined Patent Application, First Publication Nos. 2010-002870, 2010-032994, 2010-277043, 2011-13569, and 2011-128226 can be used. Specific examples of the component (F) include a polymer having a constitutional unit (f1) represented by General Formula (f1-1). As the polymer, a polymer (homopolymer) formed of only the constitutional unit (f1) represented by Formula (f1-1); a copolymer of the constitutional unit (f1) and the constitutional unit (a1); or a copolymer of the constitutional unit (f1), a constitutional unit derived from acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl-1-adamantyl (meth)acrylate is preferable, and a constitutional unit derived from 1-ethyl-1-cyclooctyl (meth)acrylate is more preferable.

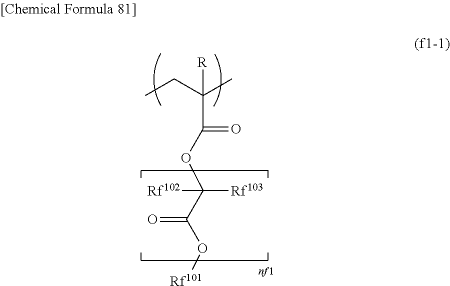

[In the formula, R has the same definition as described above, Rf¹⁰² and Rf¹⁰³ each independently represent a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other. nf¹ represents an integer of 0 to 5, and Rf¹⁰¹ represents an organic group having a fluorine atom.]

In Formula (f1-1), R bonded to the carbon atom at the α-position has the same definition as described above. It is preferable that R represents a hydrogen atom or a methyl group.

In Formula (f1-1), a fluorine atom is preferable as the halogen atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atoms as R. Among the examples, a methyl group or an ethyl group is preferable. Specific examples of the halogenated alkyl group having 1 to 5 carbon atoms for Rf¹⁰² and Rf¹⁰³ include groups in which some or all hydrogen atoms of an alkyl group having 1 to 5 carbon atoms have been substituted with halogen atoms. Among these, a fluorine atom is preferable as the halogen atom. Among these, Rf¹⁰² and Rf¹⁰³ represent preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1), nf¹ represents an integer of 0 to 5, preferably an integer of 0 to 3, and more preferably 1 or 2.

In Formula (f1-1), Rf¹⁰¹ represents an organic group having a fluorine atom and preferably a hydrocarbon group having a fluorine atom.

The hydrocarbon group having a fluorine atom may be linear, branched, or cyclic, and the number of carbon atoms thereof is preferably in a range of 1 to 20, more preferably in a range of 1 to 15, and particularly preferably in a range of 1 to 10. In the hydrocarbon group having a fluorine atom, preferably 25% or greater of the hydrogen atoms in the hydrocarbon group are fluorinated, more preferably 50% or greater thereof are fluorinated, and still more preferably 60% or greater thereof are fluorinated from the viewpoint of increasing the hydrophobicity of the resist film during immersion exposure.

Among examples, Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

The weight-average molecular weight (Mw) (in terms of polystyrene according to gel permeation chromatography) of the component (F) is preferably in a range of 1000 to 50000, more preferably in a range of 5000 to 40000, and most preferably in a range of 10000 to 30000. In a case where the weight-average molecular weight thereof is less than or equal to the upper limits of the above-described ranges, the resist composition exhibits a satisfactory solubility in a solvent for a resist enough to be used as a resist. Meanwhile, in a case where the weight-average molecular weight thereof is greater than or equal to the lower limits of the above-described ranges, water repellency of the resist film is improved.

Further, the dispersity (Mw/Mn) of the component (F) is preferably in a range of 1.0 to 5.0, more preferably in a range of 1.0 to 3.0, and most preferably in a range of 1.0 to 2.5.

In the resist composition, the component (F) may be used alone or in a combination of two or more kinds thereof.

In a case where the resist composition contains the component (F), the content of the component (F) is preferably in a range of 0.5 to 10 parts by mass and more preferably in a range of 1 to 10 parts by mass with respect to 100 parts by mass of the component (A1).

As desired, miscible additives such as additive resins, dissolution inhibitors, plasticizers, stabilizers, colorants, halation prevention agents, and dyes for improving the performance of the resist film can be added to the resist composition which is the filtration target, as appropriate.

According to the method of producing a resist composition purified product of the present aspect described above, a filtration target (a resist composition) is filtered in the step (i) by using a filter having a porous membrane that has a porous structure in which adjacent spherical cells communicate with each other and contains at least one resin skeleton selected from the group consisting of polyimide and polyamide-imide. As a result, foreign substances such as an organic substance and a metal impurity are removed from the filtration target more than ever. In particular, due to the use of the polyimide-based resin porous membrane, highly polar components and polymers, which have been difficult to be removed in the related art, are sufficiently removed from a filtration target, and among them, the highly polar polymer is specifically removed. In addition, in the step (i), a metal component as an impurity is also sufficiently removed from a filtration target. As described above, various foreign substances are efficiently removed by such a production method, and a high-purity resist composition purified product can be obtained.

Further, according to the method of producing the resist composition purified product of the present embodiment, the resist composition containing the (A1) component and the (S) component is filtered in the step (i). As the component (A1), a component containing a copolymer having a constitutional unit (a01) with an onium salt structure that generates sulfonic acid upon light exposure and a constitutional unit (a02) with an onium salt structure that generates a carboxylic acid upon light exposure is used. The sites of the onium salt structure in the constitutional unit (a01) and the constitutional unit (a02) have relatively high polarity, and a large amount of insoluble gel materials derived from a component having high polarity are likely to be contained as impurities in the copolymer immediately after synthesis having these constitutional units and the resist composition containing the copolymer.

As described above, in the method of producing the resist composition purified product according to the present embodiment, since a filter suitable for “resist composition containing a specific copolymer having the constitutional unit (a01) and the constitutional unit (a02)” which is the filtration target is adopted, impurities can be further reduced, and occurrence of defects during the formation of a resist film can be sufficiently suppressed.

The filter in the present aspect is not limited to the one that includes a porous membrane in which the communication pores 5 in which the adjacent spherical cell 1a and the spherical cell 1b communicate with each other as shown in FIG. 1 is formed, and may include a porous membrane in which, in addition to the communication pores 5 in which the spherical cells 1a and 1b communicate with each other, a cell or communication pore having another form is formed.

Examples of the cell (hereinafter, referred to as “another cell”) having another form include a cell that differs in shape or pore diameter, and examples thereof include an elliptical cell, a polyhedral cell, a spherical cell having a different pore diameter. Examples of the above-described “communication pore having another form” include a communication pore in which a spherical cell and another cell communicate with each other.

The shape or pore diameter of another cell may be appropriately determined depending on the kinds of impurities to be removed. The communication pore in which a spherical cell and another cell communicate with each other can be formed, for example, by selecting a material of the fine particle material described above or controlling the shape of the fine particles.

According to the filter including a porous membrane in which, in addition to the communication pore in which adjacent spherical cells communicate with each other, a cell or communication pore having another form is formed, it is possible to efficiently remove various foreign substances from a filtration target.

Further, the filter having a polyimide-based resin porous membrane, which is used in the filtration step, replace a filter cartridge or the like for removing impurities having a fine particle shape, which has been installed in the related art, in the supply line of the resist composition or the point of use (POU) in the semiconductor manufacturing process or can be used in combination with these. As a result, various foreign substances can be efficiently removed from a filtration target using the same device and operation as those in the related art, and a high-purity resist composition purified product can be produced.

### (Resist Pattern Forming Method)

The resist pattern forming method according to the second aspect of the present invention includes a step of obtaining a resist composition purified product, by the method of producing a resist composition purified product according to the first aspect, a step of forming a resist film on a support using the resist composition purified product, a step of exposing the resist film, and a step of developing the exposed resist film to form a resist pattern.

The resist pattern forming method according to the present aspect can be carried out, for example, as follows.

First, a resist composition purified product is obtained by the method of producing a resist composition purified product according to the first aspect. Next, the resist composition purified product is applied onto a support with a spinner or the like, and a bake (post-apply bake (PAB)) treatment is carried out, for example, under a temperature condition of 80° C. to 150° C. for 40 to 120 seconds and preferably for 60 to 90 seconds to form a resist film.

Next, the selective light exposure is carried out on the resist film, for example, by light exposure through a mask (mask pattern) having a predetermined pattern formed on the mask by using an exposure apparatus such as an electron beam drawing apparatus or an EUV exposure apparatus, or direct irradiation of the resist film for drawing with an electron beam without using a mask pattern.

Thereafter, a bake treatment (post-exposure bake (PEB)) is performed, for example, under a temperature condition of 80° C. to 150° C. for 40 to 120 seconds and preferably 60 to 90 seconds.

Next, the resist film is subjected to a developing treatment. The developing treatment is conducted using an alkali developing solution in a case of an alkali developing process and using a developing solution containing an organic solvent (organic developing solution) in a case of a solvent developing process.

After the developing treatment, it is preferable to conduct a rinse treatment. As the rinse treatment, water rinsing using pure water is preferable in a case of the alkali developing process, and rinsing using a rinse solution containing an organic solvent is preferable in a case of the solvent developing process.

In a case of the solvent developing process, after the developing treatment or the rinse treatment, the developing solution or the rinse solution attached onto the pattern may be removed by a treatment using a supercritical fluid.

After the developing treatment or the rinse treatment, drying is conducted. As desired, a bake treatment (post-bake) may be conducted after the developing treatment.

The support is not particularly limited and a known support of the related art can be used, and examples thereof include a substrate for an electronic component and a substrate on which a predetermined wiring pattern has been formed. Specific examples thereof include a metal substrate such as a silicon wafer, copper, chromium, iron, or aluminum; and a glass substrate. As the materials of the wiring pattern, copper, aluminum, nickel, or gold can be used.

The wavelength to be used for light exposure is not particularly limited and the exposure can be conducted using radiation such as an ArF excimer laser, a KrF excimer laser, an F₂ excimer laser, extreme ultraviolet (EUV) rays, vacuum ultraviolet rays (VUV), electron beams (EB), X-rays, and soft X-rays.

The resist pattern forming method according to the present embodiment is a useful method in which the resist film is exposed to extreme ultraviolet (EUV) rays or electron beams (EB) in the step of exposing the resist film to light.

The method for exposing the resist film to light can be a general exposure (dry exposure) conducted in air or an inert gas such as nitrogen, or liquid immersion exposure (liquid immersion lithography).

The liquid immersion exposure is an exposure method in which the region between the resist film and the lens at the lowermost position of the exposure apparatus is filled with a solvent (liquid immersion medium) in advance that has a refractive index greater than the refractive index of air, and the exposure (immersion exposure) is conducted in this state.

As the liquid immersion medium, a solvent having a refractive index greater than the refractive index of air but less than the refractive index of the resist film to be exposed is preferable, and examples thereof include water, a fluorine-based inert liquid, a silicon-based solvent, and a hydrocarbon-based solvent.

As the liquid immersion medium, water is preferably used.

As the alkali developing solution used for the developing treatment in the alkali developing process, a 0.1 to 10 mass % tetramethylammonium hydroxide (TMAH) aqueous solution is an exemplary example.

The organic solvent contained in the organic developing solution used for the developing treatment in the solvent developing process may be any solvent that is capable of dissolving the component (A1) (the component (A1) before light exposure) and can be appropriately selected from known organic solvents. Specific examples thereof include a polar solvent such as a ketone-based solvent, an ester-based solvent, an alcohol-based solvent, a nitrile-based solvent, an amide-based solvent, and an ether-based solvent, and a hydrocarbon-based solvent.

Examples of the ester-based solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, butyl butanoate, methyl 2-hydroxyisobutyrate, isoamyl acetate, isobutyl isobutyrate, and butyl propionate.

Examples of the nitrile-based solvent include acetonitrile, propionitrile, valeronitrile, and butyronitrile.

Known additives can be blended into the organic developing solution as necessary. Examples of the additive include a surfactant. The surfactant is not particularly limited, and for example, an ionic or non-ionic fluorine-based and/or silicon-based surfactant can be used.

The developing treatment can be performed according to a known developing method, and examples thereof include a method for immersing a support in a developing solution for a certain time (a dip method), a method for raising a developing solution on the surface of a support using the surface tension and maintaining the state for a certain time (a puddle method), a method for spraying a developing solution to the surface of a support (spray method), and a method for continuously ejecting a developing solution onto a support rotating at a certain rate while scanning a developing solution ejection nozzle at a certain rate (dynamic dispense method).

As the organic solvent contained in the rinse solution used for the rinse treatment after the developing treatment in the solvent developing process, a solvent that is unlikely to dissolve a resist pattern can be appropriately selected from the organic solvents described as the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used.

These organic solvents may be used alone or in combination of two or more kinds thereof. Further, an organic solvent other than the above-described solvents and water may be mixed and used.

The rinse treatment carried out using a rinse solution (washing treatment) can be performed according to a known rinse method. Examples of the method for performing the rinse treatment include a method for continuously ejecting a rinse solution onto a support rotating at a certain rate (rotary coating method), a method for immersing a support in a rinse solution for a certain time (dip method), and a method for spraying a rinse solution to the surface of a support (spray method).

According to the resist pattern forming method of the present aspect described above, since the resist composition purified product obtained by the production method according to the first aspect is used, a resist pattern having a satisfactory shape, in which occurrence of defects is further suppressed and defects such as the generation of scum or microbridge are reduced, can be formed.

The defect count for the resist pattern is determined by measuring the number of total defects (the total number of defects, unit: number) in the support using a surface defect observation device (manufactured by KLA Corporation or the like).

### (Resist Composition Purified Product)

A resist composition purified product according to a third aspect of the present invention contains a resin component (A1) whose solubility in a developing solution is changed by the action of an acid, and an organic solvent component.

The resin component (A1) contains a copolymer having a constitutional unit (a01) with an onium salt structure that generates sulfonic acid upon light exposure and a constitutional unit (a02) with an onium salt structure that generates a carboxylic acid upon light exposure.

The resist composition purified product is formed such that the number of counting target objects having a size of 0.135 μm or greater, which are counted by a light scattering type liquid-borne particle counter, is less than 0.1 particles/mL.

The resist composition purified product according to the present aspect can be obtained by the method of producing a resist composition purified product according to the first aspect described above. The resist composition purified product obtained by the production method according to the first aspect is a composition subjected to filtration through a filter having a polyimide-based resin porous membrane to remove foreign substances. Therefore, in the resist composition purified product according to the present aspect, the number of counting target objects having a size of 0.135 μm or greater, which are counted by a light scattering type liquid-borne particle counter, is 0.1 particles/mL or less, and thus a resist composition purified product having an extremely small number of foreign substances can be realized.

Since such a resist composition purified product has an extremely small number of foreign substances, a resist pattern having a small number of defects can be formed.

As the light scattering type liquid-borne particle counter, for example, KS-41 (manufactured by RION Co., Ltd.) can be used.

In the resist composition purified product according to the present aspect, the content of a metal element selected from the group consisting of Li, Na, Mg, Al, K, Ca, Ti, V, Cr, Mn, Fe, Co, Ni, Cu, Zn, As, Ag, Cd, Sn, Ba, W, Au, and Pb is preferably less than 1.1 ppb, more preferably 1 ppb or less, still more preferably 0.9 ppb or less, even still more preferably 0.85 ppb or less, and particularly preferably 0.8 ppb or less.

The resist composition purified product according to the present aspect contains a resin component (A1) whose solubility in a developing solution is changed by the action of an acid and an organic solvent component, in which the resin component (A1) contains a copolymer having a constitutional unit (a01) with an onium salt structure that generates sulfonic acid upon light exposure and a constitutional unit (a02) with an onium salt structure that generates a carboxylic acid upon light exposure. The resist composition purified product is formed such that the content of a metal element selected from the group consisting of Li, Na, Mg, Al, K, Ca, Ti, V, Cr, Mn, Fe, Co, Ni, Cu, Zn, As, Ag, Cd, Sn, Ba, W, Au, and Pb is less than 1.1 ppb.

The resist composition purified product according to the present aspect can be obtained by the method of producing a resist composition purified product according to the first aspect described above. The resist composition purified product obtained by the production method according to the first aspect is a composition subjected to filtration through a filter having a polyimide-based resin porous membrane to remove metal impurities. Therefore, since resist composition purified product is formed such that the content of a metal element selected from the group consisting of Li, Na, Mg, Al, K, Ca, Ti, V, Cr, Mn, Fe, Co, Ni, Cu, Zn, As, Ag, Cd, Sn, Ba, W, Au, and Pb is less than 1.1 ppb, a resist composition purified product having an extremely small number of metal impurities can be realized.

Various base material components, acid generators, acid diffusion controlling agents, organic solvents, and the like, which are blended in a resist composition, contain metal components such as trace amounts of metal ion impurities. This metal component may be originally contained in the blending component; however, it may also be mixed from a chemical liquid transfer path such as a pipe or a joint of a production device. In the method of producing a resist composition purified product according to the first aspect, these metal components can be effectively removed.

In the resist composition purified product according to the present aspect, the content of the metal element is preferably 1 ppb or less, more preferably 0.9 ppb or less, and still more preferably 0.85 ppb or less.

Since the content of the metal element is extremely small in the resist composition purified product according to the present aspect, a resist pattern having a small number of defects can be formed.

The content of the metal element is the total content of Li, Na, Mg, Al, K, Ca, Ti, V, Cr, Mn, Fe, Co, Ni, Cu, Zn, As, Ag, Cd, Sn, Ba, W, Au, and Pb. The content of the metal element in the resist composition purified product can be measured using an inductively coupled plasma mass spectrometer (ICP-MS 8900, manufactured by Agilent Technologies, Inc.).

In the resist composition purified product according to a preferable aspect, the number of counting target objects having a size of 0.135 μm or greater, which are counted by a light scattering type liquid-borne particle counter, is 0.1 piece/mL or less, and the content of the metal element selected from the group consisting of Li, Na, Mg, Al, K, Ca, Ti, V, Cr, Mn, Fe, Co, Ni, Cu, Zn, As, Ag, Cd, Sn, Ba, W, Au, and Pb is less than 1.1 ppb.

It is preferable that the resist composition purified product according to the third aspect has the same composition as the resist composition that is used in the method of producing a resist composition purified product according to the first aspect described above, except that foreign substances and metal components are removed.

The resist composition purified product according to the third aspect can also be produced, for example, by a production method including the following steps (a) to (c).

Step (a): A step of filtering a resin solution prepared by dissolving a resin component for a resist (a component (A1)) in an organic solvent component through a filter having a polyimide-based resin porous membrane to obtain a resin solution purified product

Step (b): A step of mixing the resin solution purified product with another component (the component (B), the component (D), the component (E), the component (F), or the component (F) described above) to obtain a resist composition

The step (c): A step of filtering the resist composition with a filter having a polyimide-based resin porous membrane

### (Resist Pattern Forming Method)

A resist pattern forming method according to a fourth aspect of the present invention includes a step of forming a resist film on a support using the resist composition purified product according to the third aspect, a step of exposing the resist film to light, and a step of developing the resist film exposed to light to form a resist pattern.

The resist pattern forming method according to the present aspect can be carried out in the same manner as the resist pattern forming method according to the second aspect described above.

### EXAMPLES

Hereinafter, the present invention will be described in more detail based on examples, but the present invention is not limited to these examples.

### <Production of Resist Composition Purified Product>

### Examples 1 to 8 and Comparative Examples 1 to 11

The following resist compositions (1) to (7) were filtered through a filter described below to produce resist composition purified products.

### <<Preparation of Resist Composition>>

Each component listed in Table 1 was mixed and dissolved to prepare each of resist compositions (1) to (7).

TABLE 1

| | Component | Component | Component | Component |
|---|---|---|---|---|
| | (A) | (B) | (D) | (S) |
| Resist | (A)-1 | — | — | (S)-1 |
| composition (1) | [100] | | | [2500] |
| Resist | (A)-2 | — | — | (S)-1 |
| composition (2) | [100] | | | [2500] |
| Resist | (A)-3 | — | — | (S)-1 |
| composition (3) | [100] | | | [2500] |
| Resist | (A)-4 | — | — | (S)-1 |
| composition (4) | [100] | | | [2500] |
| Resist | (A)-5 | — | (D)-1 | (S)-1 |
| composition (5) | [100] | | [5] | [2500] |
| Resist | (A)-6 | (B)-1 | — | (S)-1 |
| composition (6) | [100] | [25] | | [2500] |
| Resist | (A)-7 | (B)-1 | (D)-1 | (S)-1 |
| composition (7) | [100] | [19] | [6] | [2500] |

In Table 1, each abbreviation has the following meaning. The numerical values in the brackets represent the blending amounts (parts by mass; in terms of solid content).

(A)-1: copolymer represented by Chemical Formula (A1-1), weight-average molecular weight (Mw) of 9,200, polydispersity (Mw/Mn) of 1.76, copolymer composition ratio l/m/n/o=40/40/15/5
(A)-2: copolymer represented by Chemical Formula (A1-2), weight-average molecular weight (Mw) of 9,800, polydispersity (Mw/Mn) of 1.54, and copolymerization composition ratio l/m/n/o=30/50/15/5
(A)-3: copolymer represented by Chemical Formula (A1-3), weight-average molecular weight (Mw) of 9,300, polydispersity (Mw/Mn) of 1.56, and copolymerization composition ratio l/m/n/o=30/50/15/5
(A)-4: copolymer represented by Chemical Formula (A1-4), weight-average molecular weight (Mw) of 9,900, polydispersity (Mw/Mn) of 1.54, and copolymerization composition ratio l/m/n/o=30/50/15/5

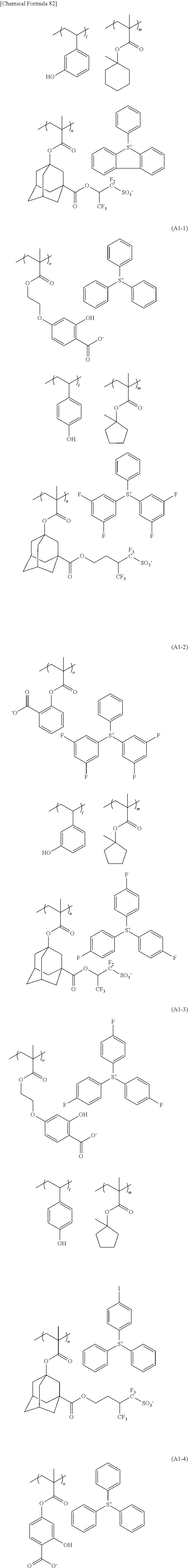

(A)-5: copolymer represented by Chemical Formula (A2-1), weight-average molecular weight (Mw) of 9,800, polydispersity (Mw/Mn) of 1.70, and copolymerization composition ratio l/m/n=35/50/15

(A)-6: copolymer represented by Chemical Formula (A2-2), weight-average molecular weight (Mw) of 6,700, polydispersity (Mw/Mn) of 1.71, and copolymer composition ratio l/m/n=45/50/5

(A)-7: copolymer represented by Chemical Formula (A2-3), weight-average molecular weight (Mw) of 5,100, polydispersity (Mw/Mn) of 1.65, and copolymerization composition ratio l/m=40/60

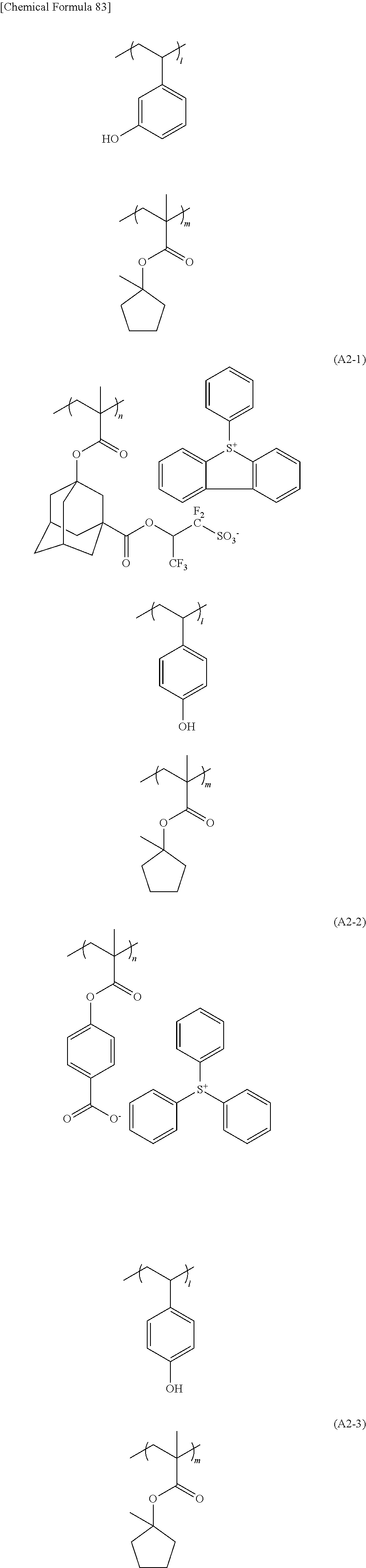

(B)-1: acid generator consisting of compound represented by Chemical Formula (B-1)

(D)-1: acid diffusion control agent consisting of compound represented by Chemical Formula (D1-1)

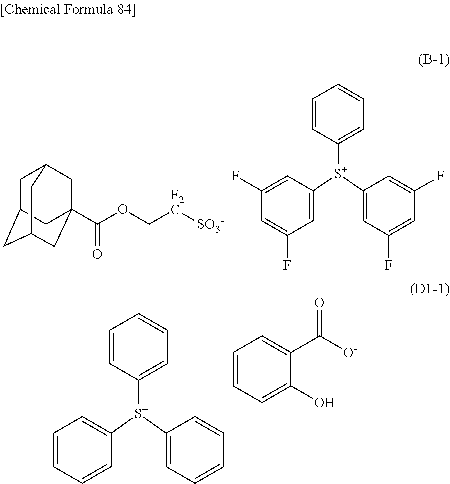

(S)-1: mixed solvent of propylene glycol monomethyl ether acetate/propylene glycol monomethyl ether=60/40 (mass ratio)

### <<Used Filter>>

Filter (F1-1): polyimide porous filter, average pore diameter of spherical cells: 300 nm, communication pores: 60 nm

Filter (F1-2): polyimide porous filter, average pore diameter of spherical cells: 100 nm, communication pores: 25 nm

Filter (F1-3): polyimide porous filter, average pore diameter of spherical cells: 80 nm, communication pores: 20 nm

Filter (F1-4): polyimide porous filter, average pore diameter of spherical cell: 50 nm, communication pore: 13 nm

The filters (F1-1) to (F1-4) were 10-inch filters including a porous membrane with a polyimide resin structure and a porous structure in which communication pores were formed by adjacent spherical cells communicating with each other.

The polyimide porous membranes in the filters (F1-1) to (F1-4) were prepared by the production method described in Japanese Unexamined Patent Application, First Publication No. 2017-68262A.

Further, in the filters (F1-1) to (F1-4), the communication pores in the polyimide porous membrane and the average pore diameter of the spherical cells were measured using a palm porometer (manufactured by PMI) according to the half-dry method (ASTM E1294-89) using perfluoropolyester (trade name, Galwick, interfacial tension value: 15.9 dyne/cm) as the test liquid at a measurement temperature of 25° C. and a measurement pressure range of 0 to 400 psi.

Filter (F2-1): filter including porous membrane made of ultrahigh-molecular-weight polyethylene (UPE) Entegris Microgard UC filter, nominal size of 1 nm

Filter (F2-2): filter including porous membrane made of polytetrafluoroethylene, nominal size of 5 nm

### Example 1

The resist composition (1) as a filtration target was filtered through the filter (F1-1) under the following filtration conditions, thereby obtaining a resist composition purified product.

Filtration conditions: filtration pressure of 0.2 MPa and filtration temperature of 23° C.

### Examples 2 to 7

Each resist composition purified product was obtained by performing the filtration in the same manner as in Example 1 except that the combination of the resist composition as the filtration object and the filter was changed.

### Example 8

### Step (ii): Washing of Filter

49 parts by mass of 7-butyrolactone, 49 parts by mass of propylene glycol monomethyl ether, 1 part by mass of acetylacetone, and 1 part by mass of lactic acid were mixed to prepare a membrane washing liquid. Next, the filter (F1-1) was immersed in 100 cm³ of the membrane washing liquid for 1 day at room temperature (23° C.). Next, the entire membrane washing liquid was removed, and the membrane was subjected to solvent replacement twice with OK73 thinner (product name, manufactured by TOKYO OHKA KOGYO CO., LTD.), and then immersed in 100 cm³ of OK73 thinner for 1 day at room temperature.

### Step (i): Filtration

The resist composition (1) as the filtration target was filtered through the filter (F1-1) after being washed in step (ii) under the following filtration conditions, thereby obtaining a resist composition purified product.

Filtration conditions: filtration pressure of 0.2 MPa and filtration temperature of 23° C.

### Comparative Examples 1 and 2

Each resist composition purified product was obtained by performing filtration in the same manner as in Example 1 except that the filter was changed.

### Comparative Examples 3 to 5

Each resist composition purified product was obtained by performing filtration in the same manner as in Example 1 except that the resist composition as the filtration target was changed.

### Comparative Examples 6 to 8

Each resist composition purified product was obtained by performing filtration in the same manner as in Comparative Example 1 except that the resist composition as the filtration target was changed.

### Comparative Examples 9 to 11

Each resist composition purified product was obtained by performing filtration in the same manner as in Comparative Example 2 except that the resist composition as the filtration target was changed.

The combinations of the resist compositions as the filtration targets, the used filters, and the production methods (filtration and filter washing) during the production of the resist composition purified product of each example are listed in Tables 2 and 3.

TABLE 2

| | Resist composition | Filter | Production method |
|---|---|---|---|
| Example 1 | (1) | (F1-1) | Filtration |
| Example 2 | (2) | (F1-1) | Filtration |
| Example 3 | (3) | (F1-1) | Filtration |
| Example 4 | (4) | (F1-1) | Filtration |
| Example 5 | (1) | (F1-2) | Filtration |
| Example 6 | (1) | (F1-3) | Filtration |
| Example 7 | (1) | (F1-4) | Filtration |
| Example 8 | (1) | (F1-1) | Washing of filter + |
| | | | filtering through filter |

TABLE 3

| | Resist | | Production |
|---|---|---|---|
| | composition | Filter | method |
| Comparative | (1) | (F2-1) | Filtration |
| Example 1 | | | |
| Comparative | (1) | (F2-2) | Filtration |
| Example 2 | | | |
| Comparative | (5) | (F1-1) | Filtration |
| Example 3 | | | |
| Comparative | (6) | (F1-1) | Filtration |
| Example 4 | | | |
| Comparative | (7) | (F1-1) | Filtration |
| Example 5 | | | |
| Comparative | (5) | (F2-1) | Filtration |
| Example 6 | | | |
| Comparative | (6) | (F2-1) | Filtration |
| Example 7 | | | |
| Comparative | (7) | (F2-1) | Filtration |
| Example 8 | | | |
| Comparative | (5) | (F2-2) | Filtration |
| Example 9 | | | |
| Comparative | (6) | (F2-2) | Filtration |
| Example 10 | | | |
| Comparative | (7) | (F2-2) | Filtration |
| Example 11 | | | |

### <Evaluation>

The content (ppb) of the metal element, the number of particles (particles/mL), and the number of defects (relative value) were determined for each resist composition purified product produced in each example.

### [Measurement of Content of Metal Element]

In the production method of each example, the content of the metal element in the resist compositions (1) to (7) before and after filter filtration was measured. Each metal compound in the resist composition was heated at 600° C., and the content of the metal compound was determined from the residual mass of the metal oxide after heating. The content of the metal element with respect to the total mass of the resist composition was calculated from the content of the metal compound. The results are listed in the columns of “content (ppb) of metal element” in Tables 4 and 5.

### [Measurement of Amount of Particles]

In the resist composition purified product produced in each example, the number of counting target objects with a size of 0.135 μm or greater was counted by a dynamic light scattering method using a light scattering type liquid-borne particle counter [manufactured by RION Co., Ltd., model number: KS-41, light source: semiconductor laser-excited solid-state laser (wavelength: 830 nm, rated output: 0.2 W), flow rate: 10 mL/min]. The counting was carried out three times, and the average value thereof was used as the counted value.

Further, the light scattering type liquid-borne particle counter was used after calibration with a polystyrene latex (PSL) standard particle solution. The results are listed in the columns of “amount (number/mL) of particles” in Tables 4 and 5.

### [Measurement of Number of Defects]

The resist composition purified product produced in each example was applied onto a 12-inch silicon substrate which had been subjected to a hexamethyldisilazane (HMIDS) treatment using a spinner, subjected to a pre-bake (PAB) treatment on a hot plate at a temperature of 80° C. for 60 seconds, and dried to form a resist film having a film thickness of 40 nm.

The number of defects (pieces/679 cm²) with a size greater than 40 nm of the obtained resist film was measured by using a surface defect observation device (Surfscan SP5 device, manufactured by KLA Corporation). The result was defined as “number of defects,” and the relative value in which the number of defects measured for the resist composition purified product manufactured in Example 1 was set to 1 is listed in Tables 4 and 5.

TABLE 4

| | | | Number of defects |
|---|---|---|---|
| | Content of | Amount of | relative value with |
| | metal element | particles | respect to number of |
| | (ppb) | (particles/mL) | defects in Example 1 |
| Example | 0.8 | 0.1 | 1.0 |
| 1 | | | |
| Example | 0.8 | 0.1 | 1.0 |
| 2 | | | |
| Example | 0.8 | 0.1 | 1.0 |
| 3 | | | |
| Example | 0.8 | 0.1 | 1.0 |
| 4 | | | |
| Example | 0.8 | 0.1 | 0.7 |
| 5 | | | |
| Example | 0.8 | 0.1 | 0.6 |
| 6 | | | |
| Example | 0.8 | 0.1 | 0.4 |
| 7 | | | |
| Example | 0.6 | 0.1 | 1.0 |
| 8 | | | |

TABLE 5

| | | | Number of defects |
|---|---|---|---|
| | Content of | Amount of | relative value with |
| | metal element | particles | respect to number of |
| | (ppb) | (particles/mL) | defects in Example 1 |
| Comparative | 1.1 | 0.1 | 1.5 |
| Example 1 | | | |
| Comparative | 1.1 | 0.5 | 3.0 |
| Example 2 | | | |
| Comparative | 0.8 | 0.1 | 1.3 |
| Example 3 | | | |
| Comparative | 0.8 | 0.1 | 1.3 |
| Example 4 | | | |
| Comparative | 0.8 | 0.1 | 1.3 |
| Example 5 | | | |
| Comparative | 1.1 | 0.1 | 1.6 |
| Example 6 | | | |
| Comparative | 1.1 | 0.1 | 1.6 |
| Example 7 | | | |
| Comparative | 1.1 | 0.1 | 1.7 |
| Example 8 | | | |
| Comparative | 1.1 | 0.5 | 3.1 |
| Example 9 | | | |
| Comparative | 1.1 | 0.5 | 3.1 |
| Example 10 | | | |
| Comparative | 1.1 | 0.5 | 3.2 |
| Example 11 | | | |

As shown in the results listed in Tables 4 and 5, it was confirmed that the resist composition purified product produced in Example 1 had a further reduced amount of metal elements and a reduced number of defects in the resist film formation, as compared with the resist composition purified product produced in Comparative Example 1.

In addition, it was confirmed that the resist composition purified product produced in Example 1 had a further reduced amount of metal elements and particles and a greatly reduced number of defects in the resist film formation, as compared with the resist composition purified product produced in Comparative Example 2.

It was confirmed that the resist composition purified product produced in Example 1 had a reduced number of defects in the resist film formation as compared with the resist composition purified product produced in Comparative Examples 3 to 5.

This is because the resist composition (1) containing the copolymer having both the constitutional unit (a01) and the constitutional unit (a02) contained a large amount of the insoluble gel substance derived from the component having a high polarity and had high adsorptivity of the insoluble gel substance in the filter (F1-1) as compared with the resist compositions (5) to (7), and thus the removal effect was considered to be excellent.

It was confirmed that the effect of reducing the number of defects by the combination of the filter (F1-1) and the resist composition (1) was maximized based on the comparison of the combination of the three different kinds of filters and the resist composition (1) (Example 1, Comparative Example 1, and Comparative Example 2), the combination of the three different kinds of filters and the resist composition (5) (Comparative Example 3, Comparative Example 6, and Comparative Example 9), the combination of the three different kinds of filters and the resist composition (6) (Comparative Example 4, Comparative Example 7, and Comparative Example 10), and the combination of the three different kinds of filters and the resist composition (7) (Comparative Example 5, Comparative Example 8, and Comparative Example 11).

While preferred embodiments of the present invention have been described and illustrated above, it should be understood that these are exemplary of the present invention and are not to be considered as limiting. Additions, omissions, substitutions, and other modifications can be made without departing from the scope of the present invention. The present invention is not limited by the description above, but is limited only by the appended claims.

### EXPLANATION OF REFERENCES

1a: Spherical cell
1b: Spherical cell
5: Communication pore

What is claimed is:

1. A method of producing a resist composition purified product, the method comprising:
a step (i) of filtering a resist composition through a filter having a porous structure in which adjacent spherical cells communicate with each other,
wherein the filter includes a porous membrane containing at least one resin selected from the group consisting of polyimide and polyamide-imide,
the resist composition contains a resin component (A1) whose solubility in a developing solution is changed by an action of an acid, and an organic solvent component, and
the resin component (A1) contains a copolymer having a constitutional unit (a01) with an onium salt structure that generates sulfonic acid upon light exposure and a constitutional unit (a02) with an onium salt structure that generates a carboxylic acid upon light exposure.

2. The method of producing a resist composition purified product according to claim 1,
wherein the constitutional unit (a01) is a constitutional unit derived from a compound represented by General Formula (a01-b), 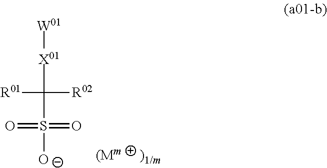
wherein W⁰¹ represents a polymerizable group-containing group. X⁰¹ represents a divalent linking group or a single bond, R⁰¹ and R⁰² each independently represent a fluorinated alkyl group, a fluorine atom, or a hydrogen atom, M^{m+} represents an m-valent onium cation, and m represents an integer of 1 or greater.

3. The method of producing a resist composition purified product according to claim 1,
wherein the constitutional unit (a02) is a constitutional unit derived from a compound represented by General Formula (a02-d), 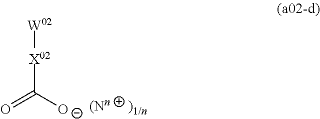
wherein W⁰² represents a polymerizable group-containing group, X⁰² represents a divalent linking group or a single bond, Nⁿ⁺ represents an n-valent onium cation, and n represents an integer of 1 or greater.

4. The method of producing a resist composition purified product according to claim 2,
wherein the constitutional unit (a01) is a constitutional unit represented by General Formula (a01-b0), 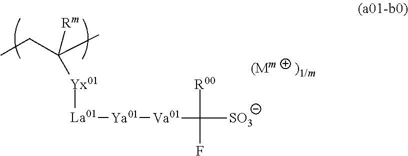
wherein R^{m} represents an alkyl group having 1 to 5 carbon atoms, a halogenated alkyl group having 1 to 5 carbon atoms, a halogen atom, or a hydrogen atom, Yx⁰¹ represents a divalent linking group or a single bond, La⁰¹ represents a hydrocarbon group which may have a substituent, Ya⁰¹ represents a divalent linking group or a single bond, Va⁰¹ represents a fluorinated alkylene group, an alkylene group, or a single bond, where both Ya⁰¹ and Va⁰¹ do not represent a single bond at the same time, R⁰⁰ represents a fluorinated alkyl group having 1 to 5 carbon atoms, a fluorine atom, or a hydrogen atom, M^{m+} represents an m-valent onium cation, and m represents an integer of 1 or greater.

5. The method of producing a resist composition purified product according to claim 3,
wherein the constitutional unit (a02) is a constitutional unit represented by General Formula (a02-d0), 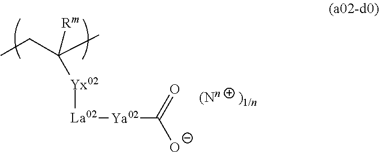
wherein R^{m} represents an alkyl group having 1 to 5 carbon atoms, a halogenated alkyl group having 1 to 5 carbon atoms, a halogen atom, or a hydrogen atom, Yx⁰² represents a divalent linking group or a single bond, La⁰² represents a hydrocarbon group which may have a substituent, Ya⁰² represents a divalent linking group or a single bond, N^{m+} represents an n-valent onium cation, and n represents an integer of 1 or greater.

6. The method of producing a resist composition purified product according to claim 1,
wherein the resin component (A1) contains a copolymer having the constitutional unit (a01), the constitutional unit (a02), and a constitutional unit (a1) containing an acid decomposable group whose polarity is increased by the action of an acid.

7. The method of producing a resist composition purified product according to claim 1,
wherein the spherical cells have an average pore diameter of 10 to 2500 nm.

8. The method of producing a resist composition purified product according to claim 1,
wherein communication pores formed by the adjacent spherical cells communicating with each other have an average pore diameter of 1 to 1000 nm.

9. The method of producing a resist composition purified product according to claim 1,
wherein the filter includes a polyimide-based resin porous membrane.

10. The method of producing a resist composition purified product according to claim 1, further comprising:
a step (ii) of washing the filter by bringing a membrane washing liquid into contact with the filter before the step (i).

11. A resist pattern forming method comprising:
a step of obtaining a resist composition purified product by the method of producing a resist composition purified product according to any one of claims 1 to 10;
a step of forming a resist film on a support using the resist composition purified product;
a step of exposing the resist film to light; and
a step of developing the resist film exposed to light to form a resist pattern.

12. A resist composition purified product comprising:
a resin component (A1) whose solubility in a developing solution is changed by an action of an acid; and
an organic solvent component,
wherein the resin component (A1) contains a copolymer having a constitutional unit (a01) with an onium salt structure that generates sulfonic acid upon light exposure and a constitutional unit (a02) with an onium salt structure that generates a carboxylic acid upon light exposure, and
the number of counting target objects with a size of 0.135 μm or greater, which is counted by a light scattering type liquid-borne particle counter, is 0.1 particles/mL or less.

13. A resist pattern forming method comprising:
a step of forming a resist film on a support using the resist composition purified product according to claim 12;
a step of exposing the resist film to light; and
a step of developing the resist film exposed to light to form a resist pattern.
